(19) United States
(12) Patent Application Publication
(10) Pub. No.: US 2017/0224777 A1
(43) Pub. Date: Aug. 10, 2017

(54) SYNERGISTIC TUMOR TREATMENT WITH IL-2, A THERAPEUTIC ANTIBODY, AND A CANCER VACCINE

(71) Applicant: Massachusetts Institute of Technology, Cambridge, MA (US)

(72) Inventors: Karl Dane WITTRUP, Chestnut Hill, MA (US); Darrell IRVINE, Arlington, MA (US); Cary Francis OPEL, Somerville, MA (US); Kelly Dare MOYNIHAN, Allston, MA (US)

(21) Appl. No.: 15/501,535

(22) PCT Filed: Aug. 12, 2015
(86) PCT No.: PCT/US2015/044927

§ 371 Date: Feb. 3, 2017

Related U.S. Application Data
(60) Provisional application No. 62/036,588, filed on Aug. 12, 2014.
(60) Provisional application No. 62/036,577, filed on Aug. 12, 2014.
(60) Provisional application No. 62/036,595, filed on Aug. 12, 2014.
(60) Provisional application No. 62/036,947, filed on Aug. 13, 2014.

(51) Int. Cl. A61K 38/20; C07K 16/40; A61K 45/06; A61K 39/385; A61K 39/39; A61K 39/395; A61K 38/38; A61K 39/00

(52) U.S. Cl. CPC A61K 38/2013; A61K 38/385; C07K 16/40; A61K 39/0011; A61K 39/385; A61K 39/39; A61K 39/39558; A61K 45/06; A61K 47/48215; A61K 2039/627; A61K 2039/6018; A61K 2039/55561; A61K 2039/575; A61K 2039/505

(57) ABSTRACT

The present invention provides a method of treating cancer with a combination of IL-2 (e.g., extended-PK IL-2), a therapeutic antibody or fragment thereof, and a cancer vaccine. The methods of the invention can be used to treat a broad range of cancer types.

### RELATED APPLICATIONS

This application claims the benefit of the priority date of U.S. Provisional Application No. 62/036595, which was filed on Aug. 12, 2014; U.S. Provisional Application No. 62/036577, which was filed on Aug. 12, 2014; U.S. Provisional Application No. 62/036947, which was filed on Aug. 13, 2014; and U.S. Provisional Application No. 62/036588, which was filed on Aug. 12, 2014. The content of this provisional application is hereby incorporated by reference in its entirety.

### GOVERNMENT FUNDING

This invention was made with government support under Grant No. CA174795 awarded by the National Institutes of Health. The government has certain rights in the invention.

### BACKGROUND

Combinatorial therapy has become an important development in cancer treatment. However, determining which therapies are more effective when combined is not intuitive. Several monotherapies have recently been developed that work synergistically when combined.

Interleukin-2 (IL-2) is a pleiotropic cytokine that activates and induces the proliferation of T cells and NK cells. To reduce toxicity and increase the efficacy of IL-2, a pharmacokinetic extending group can be added to the molecule. Additionally, combining extended half-life IL-2 and an antibody against a tumor-specific antigen shows promising results for treatment. Cancer vaccines have also become therapeutics of interest. Cancer vaccines can be used to stimulate the immune system against a specific antigen. Individually, these various therapies show promising yet limited results. However, their effectiveness together remains unexplored. Novel combination therapies are needed to more effectively combat various cancers.

### SUMMARY

The present invention is based, in part, on the discovery that administration of IL-2 attached to a pharmacokinetic modifying group (hereafter referred to as “extended-pharmacokinetic (PK) IL-2”), a therapeutic antibody, and a cancer vaccine provides synergistic tumor control and prolongs survival relative to monotherapy of either agent alone or double combinations of these three agents.

Accordingly, in one aspect, the invention provides methods of treating a hyperproliferative disorder in a subject comprising administering to the subject a therapeutically effective amount of interleukin (IL)-2; a therapeutic antibody or antibody fragment; and a cancer vaccine.

In another aspect, the invention provides a method for inhibiting growth and/or proliferation of tumor cells in a subject comprising administering to the subject an effective amount of (i) IL-2 or extended-PK IL-2; (ii) a therapeutic antibody; and (iii) a cancer vaccine, thereby inhibiting growth and/or proliferation of tumor cells in the subject.

In certain embodiments of the foregoing aspects, the IL-2 is an extended-PK IL-2. In certain embodiments of the foregoing aspects the extended-PK IL-2 comprises a fusion protein. In certain embodiments of the foregoing aspects, the fusion protein comprises an IL-2 moiety and a moiety selected from the group consisting of an immunoglobulin fragment (e.g., an immunoglobulin Fc domain), serum albumin (e.g., human serum albumin), transferrin, and Fn3, or variants thereof. In certain embodiments of the foregoing aspects, the IL-2 or extended-PK IL-2 comprises an IL-2 moiety conjugated to a non-protein polymer, such as polyethylene glycol.

In certain embodiments of the foregoing aspects, the therapeutic antibody or antibody fragment recognizes a tumor antigen.

In certain embodiments of the foregoing aspects, the cancer vaccine is a population of cells immunized in vitro with a tumor antigen and administered to the subject. In certain embodiments of the foregoing aspects, the cancer vaccine is an amphiphilic peptide conjugate comprising a tumor-associated antigen, and a lipid component, and optionally a linker, wherein the amphiphilic peptide conjugate binds albumin under physiological conditions. In certain embodiments of the foregoing aspects, the tumor-associated antigen is conjugated to a lipid via a linker, wherein the linker is selected from hydrophilic polymers, a string of hydrophilic amino acids, polysaccharides or a combination thereof. In certain embodiments of the foregoing aspects, the linker comprises “N” consecutive polyethylene glycol units, wherein N is between 25-50. In certain embodiments of the foregoing aspects, the lipid is a diacyl lipid. In certain embodiments of the foregoing aspects, the cancer vaccine further comprises an adjuvant, such as an amphiphilic oligonucloetide conjugate comprising an immunostimulatory oligonucelotide conjugated to a lipid (e.g., a diacyl lipid) with or without a linker (e.g., an oligonucleotide linker which comprises, e.g., “N” consecutive guanines, wherein N is between 0-2), and optionally a polar compound, wherein the conjugate binds albumin under physiological conditions. In certain embodiments of the foregoing aspects, the molecular adjuvant is an immunostimulatory oligonucleotide (e.g., an oligonucleotide comprising CpG) that can bind a pattern recognition receptor. In certain embodiments of the foregoing aspects, the immunostimulatory oligonucelotide is a ligand for a toll-like receptor.

In any of the foregoing aspects, the methods further comprise administering an immune checkpoint blocker, such an antibody or antibody fragment targeting PD-1, PD-L1, CTLA-4, TIM3, LAGS, or a member of the B7 family. In one embodiment, the immune checkpoint blocker is an antibody or antibody fragment thereof targeting PD-1. In another embodiment, the immune checkpoint blocker is an antibody or antibody fragment targeting CTLA4.

In certain embodiments, an antagonist of VEGF is administered in place of an immune checkpoint blocker. In a further embodiment, the antagonist of VEGF is an antibody or antibody fragment thereof that binds VEGF, an antibody or antibody fragment thereof that binds VEGF receptor, a small molecule inhibitor of the VEGF receptor tyrosine kinases, a dominant negative VEGF, or a VEGF receptor.

In certain embodiments of the foregoing aspects, the IL-2 or extended-PK IL-2, therapeutic antibody or fragment, cancer vaccine, and optional immune checkpoint blocker are administered simultaneously or sequentially.

In certain embodiments of the foregoing aspects, the IL-2 or extended-PK IL-2, therapeutic antibody or fragment, cancer vaccine, and optional antagonist of VEGF are administered simultaneously or sequentially.

In certain embodiments of the foregoing aspects, the subject has a tumor. In certain embodiments of the foregoing aspects, the invention provides a method for increasing the number of interferon gamma expressing CD8+ T cells in a tumor. In another aspect, the invention provides a method for increasing the ratio of CD8+ T cells to T regulatory cells in the tumor.

In certain embodiments of the foregoing aspects, the hyperproliferative disorder treated by the methods disclosed herein is cancer, such as melanoma, leukemia, lymphoma, lung cancer, breast cancer, prostate cancer, ovarian cancer, colon cancer, mesothelioma, renal cell carcinoma, and brain cancer.

### BRIEF DESCRIPTION OF THE DRAWINGS

FIG. 1A is a schematic of the melanoma in vivo model depicting components for tumor establishment and treatment, as described in the Examples. B16F10 melanoma cells were injected into C57BL/6 mice. After tumor establishment, treatment was administered. Treatment included a combination of an amphiphile vaccine against Trp-2, a tumor specific antibody against Trp-1 (TA99), and MSA-IL-2.

FIG. 1B is a schematic depicting the treatment regimen administered after tumor establishment, as described in the Examples. 1×10⁶ B16F10 melanoma cells were injected subcutaneously into C57BL/6 mice, and 8, 15, and 22 days after tumor injection, immunotherapy support and a vaccine was administered to the mice. Additional immunotherapy support was administered at days 29 and 35 after tumor injection. Blood was collected prior to immunotherapy support and an assay to measure Trp-2 reactive T-cells was performed (marked as “x” on the time line).

FIGS. 2A and 2B depict the effects of various combination therapies including vehicle, TA99 antibody, MSA-IL2, and/or amphiphile vaccine on tumor control. FIG. 2A shows tumor size trajectories. FIG. 2B shows a Kaplan-Meier survival plot.

FIG. 3 is an image of mice treated with the Trp-2 vaccine alone (control) or MSA-IL-2+TA99+Trp-2 vaccine. Images were taken of surviving mice 55 days after tumor inoculation. Vitiligo is observed in mice treated with the combination therapy.

FIG. 4 is a plot representative of the Trp-2 assay, where the gating and subsequent percentage of Trp-2 reactive CD8+ T cells is shown. Peripheral blood mononuclear cells were removed from the mice and stimulated with Trp-2 antigen. The response to Trp-2 was measured by counting the number of IFNγ producing cells via FACS in CD8+ T cells.

FIG. 5 is a line graph showing the temporal change in percentage of IFNγ producing CD8+ T cells after tumor inoculation. Peripheral blood mononuclear cells were isolated from mice throughout the duration of treatment. Trp-2 was used to stimulate the cells to determine the strength of the IFNγ response, a reflection of memory T cells.

FIG. 6 is a schematic representation of lipid-oligonucleotide conjugates.

FIG. 7 is a schematic representation of a lipid-peptide conjugate, as described herein.

FIG. 8A is a schematic of the melanoma in vivo model depicting components for tumor establishment and treatment, as described in the Examples. B16F10 melanoma cells were injected into C57BL/6 mice. After tumor establishment, the indicated treatments (e.g., amphiphile vaccine against Trp-2, a tumor-specific antibody against Trp-1 (TA99), MSA-IL-2, or an immune checkpoint blocker antibody targeting PD-1, or combinations thereof) were administered.

FIG. 8B is a schematic depicting the treatment regimen administered after tumor establishment, as described in the Examples. 1×10⁶ B16F10 melanoma cells were injected subcutaneously into C57BL/6 mice; 8, 15, and 22 days after tumor injection, immunotherapy support and/or a vaccine was administered to the mice. Additional immunotherapy support was administered at days 29 and 35 after tumor injection. Blood was collected prior to immunotherapy support and an assay to measure Trp-2 reactive T-cells was performed (marked as “x” on the time line).

FIGS. 9A and 9B depict the effects of various combination therapies including vehicle, anti-PD-1 antibody, TA99 antibody, MSA-IL2, and amphiphile vaccine, and combinations thereof, on tumor control. Tumor size trajectories are shown in FIG. 9A. Kaplan-Meier survival plots are shown in FIG. 9B.

FIG. 10 is a graph depicting the percentage of rejection of secondary tumor challenge. 75 days after initial tumor injection, B16F10 cells were injected into the same mice to “rechallenge” them with tumor cells.

FIG. 11 is an image of control mice and mice treated with a combination of MSA-IL-2, TA99 antibody, anti-PD-1 antibody, and/or vaccine. Images were taken of surviving mice 55 days after tumor inoculation. Vitiligo is observed in mice treated with the combination therapies.

FIG. 12 is a graph depicting the percentage of CD8+ T cells that produce IFNγ after the 1st treatment (i.e., 14 days after tumor inoculation, 6 days after the 1^{st} treatment).

. 13 is a line graph showing the temporal change in percentage of IFNγ-producing CD8+ T cells after tumor inoculation. Peripheral blood mononuclear cells were isolated from mice throughout the duration of treatment. Trp-2 was used to stimulate cells in order to determine the strength of the IFNγ response, a reflection of the T cell response induced by the vaccine.

FIG. 14 is a line graph showing the temporal change in percentage of IFNγ-producting CD8+ T cells after rechallenge with B16F10 cells in mice with or without a primary tumor. Peripheral blood mononuclear cells were isolated from mice throughout the duration of treatment. Trp-2 was used to stimulate the cells to determine the strength of the IFNγ response, a reflection of the T cell response induced by the vaccine.

FIG. 15 is an image of mice with or without primary tumors treated with a combination of MSA-IL-2, TA99 antibody, anti-PD-1 antibody, and vaccine, along with untreated mice. Images were taken of surviving mice 55 days after tumor inoculation. Vitiligo is observed in mice treated with the quadruple combination therapy, with or without primary tumors.

FIG. 16 shows Kaplan-Meier survival plots depicting the effects of various immune cell depletions perfomed in mice after tumor inoculation with B16F10 cells and one day prior to treatment with a combination of MSA-IL-2, TA99 antibody, anti-PD-1 antibody, and vaccine. Neutrophils, natural killer cells (NK) and CD8+ T cells (CD8) were depleted with antibodies against Ly-6G, NK1.1, and CD8, respectively, at a dose of 400 μg administered twice a week starting one day prior to the first treatment. The role of dendritic cells was determined using Batf3−/− mice. * p<0.05 ** p<0.01 ***p<0.001

FIG. 17A is a graph depicting the number of CD8+ T cells per mg of tumor in B16F10 tumors 4 days after a single dose of the indicated combinations of MSA-IL-2, TA99 antibody, anti-PD-1 antibody, and/or vaccine, or PBS, as measured by intracellular cytokine staining.

FIG. 17B is a graph depicting the ratio of CD8+ T cells:regulatory T cells (Tregs). Along with the measurement of CD8+ T cells in FIG. 17A, Tregs in tumors were measured via flow cytometry 4 days after a single dose of the indicated combinations. *** p<0.001

FIG. 17C is a graph showing the number of neutrophils per gram of tumor in B16F10 tumors, measured the same as CD8+ T cells and Tregs.

FIG. 18 is a graph depicting the response to OVA peptide when using B16F10-OVA cells. Shown is the proportion of tetramer+ CD8+ T cells, as determined by intracellular cytokine staining at day 21.

FIG. 19 is an image of B16F10 lysate run on an SDS-PAGE gel. Serum from mice was used to probe the cell lysate for binding. Serum from untreated mice, mice treated with TA99 antibody, and mice treated with the quadruple combination of MSA-IL-2, TA99 antibody, anti-PD-1 antibody, and vaccine after secondary challenge (i.e., 100 days post initial tumor inoculation) was used.

FIG. 20 is a schematic depicting the treatment regimen administered after tumor establishment using the BRAF/PTEN mouse model, as described in the Examples. Tamoxifen was administered to the left ear of BRAF/PTEN-TG mice on three consecutive days. Treatment started 24-26 days later, when visible tumor lesions were present. A combination of MSA-IL-2, TA99 antibody, PD-1 antibody, and a vaccine was administered to the mice every 7 days for 3 treatments total. Following this, MSA-IL-2 and TA99 antibody were administered another two times with 7 days in between each treatment.

FIG. 21 shows images of ears from BRAF/PTEN-TG mice that received no treatment or a combination of MSA-IL-2, TA99 antibody, PD-1 antibody, and a vaccine, during the first 60 days of tumor establishment and treatment. Images were taken on the day of the first treatment (i.e., approximately 24-26 days after tumor induction), the fifth treatment (i.e., approximately 50 days after tumor induction), and post treatment (i.e., approximately 60 days after tumor induction).

FIG. 22 is a Kaplan-Meier plot depicting the survival of BRAF/PTEN-TG mice that received no treatment or a combination of MSA-IL-2, TA99 antibody, anti-PD-1 antibody, and a vaccine, up to 90 days post-treatment.

### DETAILED DESCRIPTION

### Overview

Various diseases are characterized by the development of progressive immunosuppression in a patient. The presence of an impaired immune response in patients with malignancies has been particularly well documented. Cancer patients and tumor-bearing mice exhibit a variety of altered immune functions such as a decrease in delayed type hypersensitivity, a decrease in lytic function and proliferative response of lymphocytes. Augmenting immune functions in cancer patients could have beneficial effects for tumor control.

In one aspect, the present invention relates to a method of treating cancer comprising administering IL-2 (e.g., extended-PK IL-2), a therapeutic antibody, a cancer vaccine, and optionally an immune checkpoint blocker. Each of these therapeutics individually target the immune system. In another aspect, the methods of the present invention prolong survival of subjects with cancer. In yet another aspect, the methods of the present invention inhibit metastases. In another aspect, the methods of the present invention reduce tumor size. In yet another aspect, the methods of the present invention inhibit the growth of tumor cells.

### Definitions

Terms used in the claims and specification are defined as set forth below unless otherwise specified. In the case of direct conflict with a term used in a parent provisional patent application, the term used in the instant application shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups {e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single- stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, “interleukin (IL)-2,” refers to a pleiotropic cytokine that activates and induces proliferation of T cells and natural killer (NK) cells. IL-2 signals by binding its receptor, IL-2R, which is comprised of alpha, beta, and gamma subunits. IL-2 signaling stimulates proliferation of antigen-activated T cells.

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include PEG, human serum albumin (HSA) binders (as disclosed in U.S. Publication Nos. 2005/0287153 and 2007/0003549, PCT Publication Nos. WO 2009/083804 and WO 2009/133208, and SABA molecules as described in US2012/094909), serum albumin (e.g., HSA), Fc or Fc fragments and variants thereof, transferrin and variants thereof, and sugars (e.g., sialic acid). Other exemplary extended-PK groups are disclosed in Kontermann et al., Current Opinion in Biotechnology 2011;22:868-876, which is herein incorporated by reference in its entirety. As used herein, an “extended-PK IL-2” refers to an IL-2 moiety in combination with an extended-PK group. In one embodiment, the extended-PK IL-2 is a fusion protein in which an IL-2 moiety is linked or fused to an extended-PK group. An exemplary fusion protein is an HSA/IL-2 fusion in which one or more IL-2 moieties are linked to HSA.

The term “extended-PK IL-2” is also intended to encompass IL-2 mutants with mutations in one or more amino acid residues that enhance the affinity of IL-2 for one or more of its receptors, for example, CD25. In one embodiment, the IL-2 moiety of extended-PK IL-2 is wild-type IL-2. In another embodiment, the IL-2 moiety is a mutant IL-2 which exhibits greater affinity for CD25 than wild-type IL-2. When a particular type of extended-PK group is indicated, such as HSA-IL-2, it should be understood that this encompasses both HSA or MSA fused to a wild-type IL-2 moiety or HSA or MSA fused to a mutant IL-2 moiety.

In certain aspects, the extended-PK IL-2, suitable for use in the methods disclosed herein, can employ one or more “linker domains,” such as polypeptide linkers. As used herein, the term “linker” or “linker domain” refers to a sequence which connects two or more domains (e.g., the PK moiety and IL-2) in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect an IL-2 moiety to an Fc domain. Preferably, such polypeptide linkers can provide flexibility to the polypeptide molecule. In certain embodiments the polypeptide linker is used to connect (e.g., genetically fuse) one or more Fc domains and/or IL-2.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, the term “Fc region” refers to the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, an Fc domain can also be referred to as “Ig” or “IgG.” In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. A human IgG1 constant region can be found at Uniprot P01857 and in Table 1 (i.e., SEQ ID NO: 1). The Fc domain of human IgG1 can be found in Table 1 (i.e., SEQ ID NO: 2). The Fc domain encompasses native Fc and Fc variant molecules. As with Fc variants and native Fe's, the term Fc domain includes molecules in monomeric or multimeric form, whether digested from whole antibody or produced by other means. The assignment of amino acid residue numbers to an Fc domain is in accordance with the definitions of Kabat. See, e.g., Sequences of Proteins of Immunological Interest (Table of Contents, Introduction and Constant Region Sequences sections), 5th edition, Bethesda, Md.:NIH vol. 1:647-723 (1991); Kabat et al.,“Introduction” Sequences of Proteins of Immunological Interest, US Dept of Health and Human Services, NIH, 5th edition, Bethesda, Md. vol. 1:xiii-xcvi (1991); Chothia & Lesk, J. Mol. Biol. 196:901-917 (1987); Chothia et al., Nature 342:878-883 (1989), each of which is herein incorporated by reference for all purposes.

As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide of the invention may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions. A polypeptide can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain embodiments, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. In certain embodiments, a polypeptide consists of, consists essentially of, or comprises an amino acid sequence selected from SEQ ID NOs: 4, 6, 8, 10, 12, 16, 18, 20, 22, 24, 26, 28, 30, 32, and 34. In certain embodiments, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from SEQ ID NOs: 4, 6, 8, 10, 12, 16, 18, 20, 22, 24, 26, 28, 30, 32, and 34. In certain embodiments, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from SEQ ID NOs: 4, 6, 8, 10, 12, 16, 18, 20, 22, 24, 26, 28, 30, 32, and 34. In certain embodiments, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from SEQ ID NOs: 4, 6, 8, 10, 12, 16, 18, 20, 22, 24, 26, 28, 30, 32, and 34.

In certain embodiments, the peptides are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like. In certain embodiments, the nucleotide sequence of the invention comprises, consists of, or consists essentially of, a nucleotide sequence selected from SEQ ID NOs: 3, 5, 7, 9, 11, 15, 17, 19, 21, 23, 25, 27, 29, 31, and 33. In certain embodiments, a nucleotide sequence includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence set forth in SEQ ID NOs: 3, 5, 7, 9, 11, 15, 17, 19, 21, 23, 25, 27, 29, 31, and 33. In certain embodiments, a nucleotide sequence includes a contiguous nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous nucleotide sequence set forth in SEQ ID NOs: 3, 5, 7, 9, 11, 15, 17, 19, 21, 23, 25, 27, 29, 31, and 33. In certain embodiments, a nucleotide sequence includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence set forth in SEQ ID NOs: 3, 5, 7, 9, 11, 15, 17, 19, 21, 23, 25, 27, 29, 31, and 33.

It will also be understood by one of ordinary skill in the art that the IL-2 (e.g., extended-PK IL-2) suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The IL-2 (e.g., extended-PK IL-2) and Fc molecules suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain embodiments, a string of amino acids can be replaced with a structurally similar string that differs in order and/or composition of side chain family members. Alternatively, in certain embodiments, mutations may be introduced randomly along all or part of a coding sequence, such as by saturation mutagenesis, and the resultant mutants can be incorporated into binding polypeptides of the invention and screened for their ability to bind to the desired target.

The term “ameliorating” refers to any therapeutically beneficial result in the treatment of a disease state, e.g., cancer, including prophylaxis, lessening in the severity or progression, remission, or cure thereof.

The term “in vivo” refers to processes that occur in a living organism. The term “mammal” or “subject” or “patient” as used herein includes both humans and non-humans and includes, but is not limited to, humans, non-human primates, canines, felines, murines, bovines, equines, and porcines.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As used herein, the term “gly-ser polypeptide linker” refers to a peptide that consists of glycine and serine residues. An exemplary gly-ser polypeptide linker comprises the amino acid sequence Ser(Gly₄Ser)n. In certain embodiments, n=1. In certain embodiments, n=2. In certain embodiments, n=3, i.e., Ser(Gly₄Ser)3. In certain embodiments, n=4, i.e., Ser(Gly₄Ser)4. In certain embodiments, n=5. In certain embodiments, n=6. In certain embodiments, n=7. In certain embodiments, n=8. In certain embodiments, n=9. In certain embodiments, n=10. Another exemplary gly-ser polypeptide linker comprises the amino acid sequence (Gly₄Ser)n. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises the amino acid sequence (Gly₃Ser)n. certain embodiments, n=1. In certain embodiments, n=2. In certain embodiments, n=3. In certain embodiments, n=4. In certain embodiments, n=5. In certain embodiments, n=6.

As used herein, the terms “linked,” “fused”, or “fusion” are used interchangeably. These terms refer to the joining together of two or more elements or components or domains, by whatever means including chemical conjugation or recombinant means. Methods of chemical conjugation (e.g., using heterobifunctional crosslinking agents) are known in the art.

As used herein, “half-life” refers to the time taken for the serum or plasma concentration of a polypeptide to reduce by 50%, in vivo, for example due to degradation and/or clearance or sequestration by natural mechanisms. The extended-PK IL-2 suitable for use in the methods disclosed herein is stabilized in vivo and its half-life increased by, e.g., fusion to an Fc region, fusion to serum albumin (e.g., HSA or MSA), through PEGylation, or by binding to serum albumin molecules (e.g., human serum albumin) which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fc fragments of antibodies, minibodies and diabodies. Any of the therapeutic antibodies known in the art to be useful for cancer therapy can be used in the combination therapy suitable for use in the methods disclosed herein. Therapeutic antibodies may be monoclonal antibodies or polyclonal antibodies. In preferred embodiments, the therapeutic antibodies target cancer antigens.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, “synergy” or “synergistic effect” with regard to an effect produced by two or more individual components refers to a phenomenon in which the total effect produced by these components, when utilized in combination, is greater than the sum of the individual effects of each component acting alone.

The term “sufficient amount” or “amount sufficient to” means an amount sufficient to produce a desired effect, e.g., an amount sufficient to reduce the size of a tumor.

The term “therapeutically effective amount” is an amount that is effective to ameliorate a symptom of a disease. A therapeutically effective amount can be a “prophylactically effective amount” as prophylaxis can be considered therapy.

As used herein, “combination therapy” embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, “cancer vaccine” refers to a treatment that induces the immune system to attack cells with one or more tumor associated antigens. The vaccine can treat existing cancer (e.g., therapeutic cancer vaccine) or prevent the development of cancer in certain individuals (e.g., prophylactic cancer vaccine). The vaccine creates memory cells that will recognize tumor cells with the antigen and therefore prevent tumor growth. In certain embodiments, the cancer vaccine comprises an immunostimulatory oligonucleotide.

As used herein, an “immunostimulatory oligonucleotide” is an oligonucleotide that can stimulate (e.g., induce or enhance) an immune response.

As used herein, “CG oligodeoxynucleotides (CG ODNs)”, also referred to as “CpG ODNs”, are short single-stranded synthetic DNA molecules that contain a cytosine nucleotide (C) followed by a guanine nucleotide (G). In certain embodiments, the immunostimulatory oligonucleotide is a CG ODN.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

The term “T cell” refers to a CD4+ T cell or a CD8+ T cell. The term T cell encompasses TH1 cells, TH2 cells and TH17 cells.

The term “T cell cytoxicity” includes any immune response that is mediated by CD8+ T cell activation. Exemplary immune responses include cytokine production, CD8+ T cell proliferation, granzyme or perforin production, and clearance of an infectious agent.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773 (SEQ ID NO: 38).

“Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7 (SEQ ID NO: 39).

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410 (SEQ ID NO: 40):

“Lymphocyte Activation Gene-3 (LAG3)” is an inhibitory receptor associated with inhibition of lymphocyte activity by binding to MHC class II molecules. This receptor enhances the function of Treg cells and inhibits CD8+ effector T cell function. The term “LAG3” as used herein includes human LAG3 (hLAG3), variants, isoforms, and species homologs of hLAG3, and analogs having at least one common epitope. The complete hLAG3 sequence can be found under GenBank Accession No. P18627 (SEQ ID NO: 41).

“T Cell Membrane Protein-3 (TIM3)” is an inhibitory receptor involved in the inhibition of lymphocyte activity by inhibition of T_{H}1 cells responses. Its ligand is galectin 9, which is upregulated in various types of cancers. The term “TIM3” as used herein includes human TIM3 (hTIM3), variants, isoforms, and species homologs of hTIM3, and analogs having at least one common epitope. The complete hTIM3 sequence can be found under GenBank Accession No. Q8TDQo (SEQ ID NO: 42).

The “B7 family” refers to inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406 (SEQ ID NOs: 43 and 44) respectively.

Vascular Endothelial Growth Factor (VEGF)” is a secreted disulfide-linked homodimer that selectively stimulates endothelial cells to proliferate, migrate, and produce matrix-degrading enzymes, all of which are processes required for the formation of new vessels. In addition to being the only known endothelial cell specific mitogen, VEGF is unique among angiogenic growth factors in its ability to induce a transient increase in blood vessel permeability to macromolecules. The term “VEGF” or “VEGF-A” is used to refer to the 165-amino acid human vascular endothelial cell growth factor and related 121-, 145-, 189-, and 206-amino acid human vascular endothelial cell growth factors, as described by, e.g., Leung et al. Science, 246: 1306 (1989), and Houck et al. Mol. Endocrin., 5: 1806 (1991), together with the naturally occurring allelic and processed forms thereof. VEGF-A is part of a gene family including VEGF-B, VEGF-C, VEGF-D, VEGF-E, VEGF-F, and P1GF. VEGF-A primarily binds to two high affinity receptor tyrosine kinases, VEGFR-1 (Fit-1) and VEGFR-2 (Flk-1/KDR), the latter being the major transmitter of vascular endothelial cell mitogenic signals of VEGF-A.

As used herein, “immune checkpoint” refers to co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG3 and MHC class II molecules. In certain embodiments, the inhibitory signal is the interaction between TIM3 and galectin 9.

As used herein, “immune checkpoint blocker” refers to a molecule that totally or partially reduces, inhibits, interferes with or modulates one or more checkpoint proteins. In certain embodiments, the immune checkpoint blocker prevents inhibitory signals associated with the immune checkpoint. In certain embodiments, the immune checkpoint blocker is an antibody, or fragment thereof that disrupts inhibitory signaling associated with the immune checkpoint. In certain embodiments, the immune checkpoint blocker is a small molecule that disrupts inhibitory signaling. In certain embodiments, the immune checkpoint blocker is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint blocker is an antibody, or fragment thereof, that prevents the interaction between CTLA-4 and CD80 or CD86. In certain embodiments, the immune checkpoint blocker is an antibody, or fragment thereof, that prevents the interaction between LAG3 and its ligands, or TIM-3 and its ligands. The checkpoint blocker may also be in the form of the soluble form of the molecules (or variants thereof) themselves, e.g., a soluble PD-L1 or PD-L1 fusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

It must be noted that, as used in the specification and the appended claims, the singular forms “a,” “an” and “the” include plural referents unless the context clearly dictates otherwise.

### IL-2 and Extended-PK IL-2

Interleukin-2 (IL-2) is a cytokine that induces proliferation of antigen-activated T cells and stimulates natural killer (NK) cells. The biological activity of IL-2 is mediated through a multi-subunit IL-2 receptor complex (IL-2R) of three polypeptide subunits that span the cell membrane: p55 (IL-2Rα, the alpha subunit, also known as CD25 in humans), p75 (IL-2RP, the beta subunit, also known as CD 122 in humans) and p64 (IL-2Rγ, the gamma subunit, also known as CD 132 in humans). T cell response to IL-2 depends on a variety of factors, including: (1) the concentration of IL-2; (2) the number of IL-2R molecules on the cell surface; and (3) the number of IL-2R occupied by IL-2 (i.e., the affinity of the binding interaction between IL-2 and IL-2R (Smith, “Cell Growth Signal Transduction is Quantal” In Receptor Activation by Antigens, Cytokines, Hormones, and Growth Factors 766:263-271, 1995)). The IL-2:IL-2R complex is internalized upon ligand binding and the different components undergo differential sorting. IL-2Rα is recycled to the cell surface, while IL-2 associated with the IL-2:IL-2RPγ complex is routed to the lysosome and degraded. When administered as an intravenous (i.v.) bolus, IL-2 has a rapid systemic clearance (an initial clearance phase with a half-life of 12.9 minutes followed by a slower clearance phase with a half-life of 85 minutes) (Konrad et al., Cancer Res. 50:2009-2017, 1990).

However, in certain embodiments, IL-2 therapy, such as systemic IL-2 is administered to a subject in an effective amount in combination with a therapeutic antibody, a cancer vaccine, and optionally an immune checkpoint blocker.

Outcomes of systemic IL-2 administration in cancer patients are far from ideal. While 15 to 20 percent of patients respond objectively to high-dose IL-2, the great majority do not, and many suffer severe, life-threatening side effects, including nausea, confusion, hypotension, and septic shock. The severe toxicity associated with high-dose IL-2 treatment is largely attributable to the activity of natural killer (NK) cells. NK cells express the intermediate-affinity receptor, IL-2RPγ_{c}, and thus are stimulated at nanomolar concentrations of IL-2, which do in fact result in patient sera during high-dose IL-2 therapy. Attempts to reduce serum concentration, and hence selectively stimulate IL-2RaPγ_{c}-bearing cells, by reducing dose and adjusting dosing regimen have been attempted, and while less toxic, such treatments were also less efficacious. Given the toxicity issues associated with high dose IL-2 cancer therapy, numerous groups have attempted to improve anti-cancer efficacy of IL-2 by simultaneously administering therapeutic antibodies. Yet, such efforts have been largely unsuccessful, yielding no additional or limited clinical benefit compared to IL-2 therapy alone. Accordingly, novel IL-2 therapies are needed to more effectively combat various cancers.

Applicants recently discovered that the ability of IL-2 to control tumors in various cancer models could be substantially increased by attaching IL-2 to a pharmacokinetic modifying group. The resulting molecule, hereafter referred to as “extended-pharmacokinetic (PK) IL-2,” has a prolonged circulation half-life relative to free IL-2. The prolonged circulation half-life of extended-PK IL-2 permits in vivo serum IL-2 concentrations to be maintained within a therapeutic range, leading to the enhanced activation of many types of immune cells, including T cells. Because of its favorable pharmacokinetic profile, extended-PK IL-2 can be dosed less frequently and for longer periods of time when compared with unmodified IL-2. Extended-PK IL-2 is described in detail in International Patent Application NO. PCT/US2013/042057, filed May 21, 2013, and claiming the benefit of priority to U.S. Provisional Patent Application No. 61/650,277, filed May 22, 2012. The entire contents of the foregoing applications are incorporated by reference herein.

### 1. IL-2 and Mutants Thereof

In certain embodiments, an effective amount of human IL-2 is administered systemically. In some embodiments, an effective amount of an extended-PK IL-2 is administered systemically. In one embodiment, the IL-2 is a human recombinant IL-2 such as Proleukin® (aldesleukin). Proleukin® is a human recombinant interleukin-2 product produced in E. coli. Proleukin® differs from the native interleukin-2 in the following ways: a) it is not glycosylated; b) it has no N-terminal alanine; and c) it has serine substituted for cysteine at amino acid positions 125. Proleukin® exists as biologicially active, non-covalently bound microaggregates with an average size of 27 recombinant interleukin-2 molecules. Proleukin® (aldesleukin) is administered by intravenous infusion. In some aspects, the IL-2 portion of the extended-PK IL-2 is wild-type IL-2 (e.g., human IL-2 in its precursor form (SEQ ID NO: 32) or mature IL-2 (SEQ ID NO: 34)).

In certain embodiments, the extended-PK IL-2 is mutated such that it has an altered affinity (e.g., a higher affinity) for the IL-2R alpha receptor compared with unmodified IL-2.

Site-directed mutagenesis can be used to isolate IL-2 mutants that exhibit high affinity binding to CD25, i.e., IL-2Rα, as compared to wild-type IL-2. Increasing the affinity of IL-2 for IL-2α at the cell surface will increase receptor occupancy within a limited range of IL-2 concentration, as well as raise the local concentration of IL-2 at the cell surface.

In certain embodiments, the invention features IL-2 mutants, which may be, but are not necessarily, substantially purified and which can function as high affinity CD25 binders. IL-2 is a T cell growth factor that induces proliferation of antigen-activated T cells and stimulation of NK cells. Exemplary IL-2 mutants which are high affinity binders include those described in WO2013/177187A2 (herein incorporated by reference in its entirety), such as those with amino acid sequences set forth in SEQ ID NOs: 6, 22, 24, 26, 28, and 30. Further exemplary IL-2 mutants with increased affinity for CD25 are disclosed in U.S. Pat. No. 7,569,215, the contents of which are incorporated herein by reference. In one embodiment, the IL-2 mutant does not bind to CD25, e.g., those with amino acid sequences set forth in SEQ ID NOs: 8 and 10.

IL-2 mutants include an amino acid sequence that is at least 80% identical to SEQ ID NO: 32 or 34 that bind CD25. For example, an IL-2 mutant can have at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) that increases the affinity for the alpha subunit of the IL-2 receptor relative to wild-type IL-2. It should be understood that mutations identified in mouse IL-2 may be made at corresponding residues in full length human IL-2 (nucleic acid sequence (accession: NM000586) of SEQ ID NO: 31; amino acid sequence (accession: P60568) of SEQ ID NO: 32) or human IL-2 without the signal peptide (nucleic acid sequence of SEQ ID NO: 33; amino acid sequence of SEQ ID NO: 34). Accordingly, in certain embodiments, the IL-2 moiety of the extended-PK IL-2 is human IL-2. In other embodiments, the IL-2 moiety of the extended-PK IL-2 is a mutant human IL-2.

IL-2 mutants can be at least or about 50%, at least or about 65%, at least or about 70%, at least or about 80%, at least or about 85%, at least or about 87%, at least or about 90%, at least or about 95%, at least or about 97%, at least or about 98%, or at least or about 99% identical in amino acid sequence to wild-type IL-2 (in its precursor form or, preferably, the mature form). The mutation can consist of a change in the number or content of amino acid residues. For example, the IL-2 mutants can have a greater or a lesser number of amino acid residues than wild-type IL-2. Alternatively, or in addition, IL-2 mutants can contain a substitution of one or more amino acid residues that are present in the wild-type IL-2.

By way of illustration, a polypeptide that includes an amino acid sequence that is at least 95% identical to a reference amino acid sequence of SEQ ID NO: 32 or 34 is a polypeptide that includes a sequence that is identical to the reference sequence except for the inclusion of up to five alterations of the reference amino acid of SEQ ID NO: 32 or 34. For example, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino (N-) or carboxy (C-) terminal positions of the reference amino acid sequence or anywhere between those terminal positions, interspersed either individually among residues in the reference sequence or in one or more contiguous groups within the reference sequence.

The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations can be at amino acid residues that contact IL-2Rα.

In general, the polypeptides suitable for use in the methods disclosed herein will be synthetic, or produced by expression of a recombinant nucleic acid molecule. In the event the polypeptide is an extended-PK IL-2 (e.g., a fusion protein containing at least IL-2 and a heterologous polypeptide, such as a hexa-histidine tag or hemagglutinin tag or an Fc region or human serum albumin), it can be encoded by a hybrid nucleic acid molecule containing one sequence that encodes IL-2 and a second sequence that encodes all or part of the heterologous polypeptide.

The techniques that are required to make IL-2 mutants are routine in the art, and can be performed without resort to undue experimentation by one of ordinary skill in the art. For example, a mutation that consists of a substitution of one or more of the amino acid residues in IL-2 can be created using a PCR-assisted mutagenesis technique (e.g., as known in the art and/or described herein for the creation of IL-2 mutants). Mutations that consist of deletions or additions of amino acid residues to an IL-2 polypeptide can also be made with standard recombinant techniques. In the event of a deletion or addition, the nucleic acid molecule encoding IL-2 is simply digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

In addition to generating IL-2 mutants via expression of nucleic acid molecules that have been altered by recombinant molecular biological techniques, IL-2 mutants can be chemically synthesized. Chemically synthesized polypeptides are routinely generated by those of skill in the art.

As noted above, IL-2 can also be prepared as fusion or chimeric polypeptides that include IL-2 and a heterologous polypeptide (i.e., a polypeptide that is not IL-2). The heterologous polypeptide can increase the circulating half-life of the chimeric polypeptide in vivo, and may, therefore, further enhance the properties of IL-2. As discussed in further detail infra, the polypeptide that increases the circulating half-life may be serum albumin, such as human or mouse serum albumin.

In certain embodiments, the chimeric polypeptide can include IL-2 and a polypeptide that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In certain embodiments, the chimeric polypeptide further comprises a C-terminal c-myc epitope tag.

Chimeric polypeptides can be constructed using no more than conventional molecular biological techniques, which are well within the ability of those of ordinary skill in the art to perform.

### A. Nucleic Acid Molecules Encoding IL-2

IL-2, either alone or as a part of a chimeric polypeptide, can be obtained by expression of a nucleic acid molecule. Thus, nucleic acid molecules encoding polypeptides containing IL-2 or an IL-2 mutant are considered suitable for use in the methods disclosed herein, such as those with nucleic acid sequences set forth in SEQ ID NOs: 3, 5, 7, 9, 11, 15, 17, 19, 21, 23, 25, 27, 29, 31, and 33. Just as IL-2 mutants can be described in terms of their identity with wild-type IL-2, the nucleic acid molecules encoding them will necessarily have a certain identity with those that encode wild-type IL-2. For example, the nucleic acid molecule encoding an IL-2 mutant can be at least 50%, at least 65%, preferably at least 75%, more preferably at least 85%, and most preferably at least 95% (e.g., 99%) identical to the nucleic acid encoding full length wild-type IL-2 (e.g., SEQ ID NO: 31) or wild-type IL-2 without the signal peptide (e.g., SEQ ID NO: 33).

The nucleic acid molecules suitable for use in the methods disclosed herein contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The isolated nucleic acid molecules can include fragments not found as such in the natural state. Thus, the invention encompasses use of recombinant molecules, such as those in which a nucleic acid sequence (for example, a sequence encoding an IL-2 mutant) is incorporated into a vector (e.g., a plasmid or viral vector) or into the genome of a heterologous cell (or the genome of a homologous cell, at a position other than the natural chromosomal location).

As described above, IL-2 mutants suitable for use in the methods disclosed herein may exist as a part of a chimeric polypeptide. In addition to, or in place of, the heterologous polypeptides described above, a nucleic acid molecule suitable for use in the methods disclosed herein can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-hosphotransferase (HPH), thymidine kinase (TK), lacz (encoding β-galactosidase), and xanthine guanine phosphoribosyl transferase (XGPRT). Skilled artisans will be aware of additional useful reagents, for example, of additional sequences that can serve the function of a marker or reporter.

The nucleic acid molecules suitable for use in the methods disclosed herein can be obtained by introducing a mutation into IL-2-encoding DNA obtained from any biological cell, such as the cell of a mammal. Thus, the nucleic acids (and the polypeptides they encode) can be those of a mouse, rat, guinea pig, cow, sheep, horse, pig, rabbit, monkey, baboon, dog, or cat. Typically, the nucleic acid molecules will be those of a human.

### 2. Extended-PK Groups

As described supra, IL-2 or mutant IL-2 is fused to an extended-PK group, which increases circulation half-life. Non-limiting examples of extended-PK groups are described infra. It should be understood that other PK groups that increase the circulation half-life of IL-2, or variants thereof, are also applicable to the present invention. In certain embodiments, the extended-PK group is a serum albumin domain (e.g., mouse serum albumin, human serum albumin).

In certain embodiments, the serum half-life of extended-PK IL-2 is increased relative to IL-2 alone (i.e., IL-2 not fused to an extended-PK group). In certain embodiments, the serum half-life of extended-PK IL-2 is at least 20, 40, 60, 80, 100, 120, 150, 180, 200, 400, 600, 800, or 1000% longer relative to the serum half-life of IL-2 alone. In certain embodiments, the serum half-life of the extended-PK IL-2 is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of IL-2 alone. In certain embodiments, the serum half-life of the extended-PK IL-2 is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

### A. Fc Domains

In certain embodiments, an extended-PK IL-2 includes an Fc domain, such as that with an amino acid sequence set forth in SEQ ID NO: 2. It will be understood by those in the art that epitope tags corresponding to 6X his tag on these extended-PK IL-2 with Fc domains are optional. The Fc domain does not contain a variable region that binds to antigen. Fc domains useful for producing the extended-PK IL-2 suitable for use in the methods disclosed herein may be obtained from a number of different sources. In certain embodiments, an Fc domain of the extended-PK IL-2 is derived from a human immunoglobulin. In certain embodiments, the Fc domain is from a human IgG1 constant region (SEQ ID NO: 1). The Fc domain of human IgG1 is set forth in SEQ ID NO: 2. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the Fc domain or portion thereof may be derived from any immunoglobulin class, including IgM, IgG, IgD, IgA, and IgE, and any immunoglobulin isotype, including IgG1, IgG2, IgG3, and IgG4.

In some aspects, an extended-PK IL-2 includes a mutant Fc domain. In some aspects, an extended-PK IL-2 includes a mutant, IgG1 Fc domain. In some aspects, a mutant Fc domain comprises one or more mutations in the hinge, CH2, and/or CH3 domains. In some aspects, a mutant Fc domain includes a D265A mutation.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or portions thereof) can be derived from these sequences using art recognized techniques. The genetic material obtained using any of the foregoing methods may then be altered or synthesized to obtain polypeptides suitable for use in the methods disclosed herein. It will further be appreciated that the scope of this invention encompasses alleles, variants and mutations of constant region DNA sequences.

Fc domain sequences can be cloned, e.g., using the polymerase chain reaction and primers which are selected to amplify the domain of interest. To clone an Fc domain sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7: 1509); rapid amplification of cDNA ends (Ruberti, F. et al. 1994. J. Immunol. Methods 173:33); antibody leader sequences (Larrick et al. Biochem Biophys Res Commun 1989; 160: 1250). The cloning of antibody sequences is further described in Newman et al., U.S. Pat. No. 5,658,570, filed Jan. 25, 1995, which is herein incorporated by reference.

Extended-PK IL-2 suitable for use in the methods disclosed herein may comprise one or more Fc domains (e.g., 2, 3, 4, 5, 6, 7, 8, 9, 10, or more Fc domains). In certain embodiments, the Fc domains may be of different types. In certain embodiments, at least one Fc domain present in the extended-PK IL-2 comprises a hinge domain or portion thereof. In certain embodiments, the extended-PK IL-2 suitable for use in the methods disclosed herein comprises at least one Fc domain which comprises at least one CH2 domain or portion thereof. In certain embodiments, the extended-PK IL-2 suitable for use in the methods disclosed herein comprises at least one Fc domain which comprises at least one CH3 domain or portion thereof. In certain embodiments, the extended-PK IL-2 suitable for use in the methods disclosed herein comprises at least one Fc domain which comprises at least one CH4 domain or portion thereof. In certain embodiments, the extended-PK IL-2 suitable for use in the methods disclosed herein comprises at least one Fc domain which comprises at least one hinge domain or portion thereof and at least one CH2 domain or portion thereof (e.g, in the hinge-CH2 orientation). In certain embodiments, the extended-PK IL-2 suitable for use in the methods disclosed herein comprises at least one Fc domain which comprises at least one CH2 domain or portion thereof and at least one CH3 domain or portion thereof (e.g, in the CH2-CH3 orientation). In certain embodiments, the extended-PK IL-2 suitable for use in the methods disclosed herein comprises at least one Fc domain comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, extended-PK IL-2 comprises at least one complete Fc region derived from one or more immunoglobulin heavy chains (e.g., an Fc domain including hinge, CH2, and CH3 domains, although these need not be derived from the same antibody). In certain embodiments, extended-PK IL-2 comprises at least two complete Fc domains derived from one or more immunoglobulin heavy chains. In certain embodiments, the complete Fc domain is derived from a human IgG immunoglobulin heavy chain (e.g., human IgG1).

In certain embodiments, the extended-PK IL-2 suitable for use in the methods disclosed herein comprises at least one Fc domain comprising a complete CH3 domain. In certain embodiments, the extended-PK IL-2 suitable for use in the methods disclosed herein comprises at least one Fc domain comprising a complete CH2 domain. In certain embodiments, the extended-PK IL-2 suitable for use in the methods disclosed herein comprises at least one Fc domain comprising at least a CH3 domain, and at least one of a hinge region, and a CH2 domain. In certain embodiments, the extended-PK IL-2 suitable for use in the methods disclosed herein comprises at least one Fc domain comprising a hinge and a CH3 domain. In certain embodiments, the extended-PK IL-2 suitable for use in the methods disclosed herein comprises at least one Fc domain comprising a hinge, a CH2, and a CH3 domain. In certain embodiments, the Fc domain is derived from a human IgG immunoglobulin heavy chain (e.g., human IgG1).

The constant region domains or portions thereof making up an Fc domain of the extended-PK IL-2 suitable for use in the methods disclosed herein may be derived from different immunoglobulin molecules. For example, a polypeptide suitable for use in the methods disclosed herein may comprise a CH2 domain or portion thereof derived from an IgG1 molecule and a CH3 region or portion thereof derived from an IgG3 molecule. In another example, the extended-PK IL-2 can comprise an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In certain embodiments, the extended-PK IL-2 suitable for use in the methods disclosed herein lacks one or more constant region domains of a complete Fc region, i.e., they are partially or entirely deleted. In certain embodiments, the extended-PK IL-2 suitable for use in the methods disclosed herein will lack an entire CH2 domain. In certain embodiments, the extended-PK IL-2 suitable for use in the methods disclosed herein comprise CH2 domain-deleted Fc regions derived from a vector (e.g., from IDEC Pharmaceuticals, San Diego) encoding an IgG1 human constant region domain (see, e.g., WO02/060955A2 and W002/096948A2). This exemplary vector is engineered to delete the CH2 domain and provide a synthetic vector expressing a domain-deleted IgG1 constant region. It will be noted that these exemplary constructs are preferably engineered to fuse a binding CH3 domain directly to a hinge region of the respective Fc domain.

In other constructs it may be desirable to provide a peptide spacer between one or more constituent Fc domains. For example, a peptide spacer may be placed between a hinge region and a CH2 domain and/or between a CH2 and a CH3 domain. For example, compatible constructs could be expressed wherein the CH2 domain has been deleted and the remaining CH3 domain (synthetic or unsynthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible used in the instant invention will be relatively non-immunogenic and not prevent proper folding of the Fc.

B. Changes to Fc Domains

In certain embodiments, an Fc domain employed in the extended-PK IL-2 suitable for use in the methods disclosed herein is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region.

In certain embodiments, the Fc variant comprises a substitution at an amino acid position located in a hinge domain or portion thereof. In certain embodiments, the Fc variant comprises a substitution at an amino acid position located in a CH2 domain or portion thereof. In certain embodiments, the Fc variant comprises a substitution at an amino acid position located in a CH3 domain or portion thereof. In certain embodiments, the Fc variant comprises a substitution at an amino acid position located in a CH4 domain or portion thereof.

In certain embodiments, the extended-PK IL-2 suitable for use in the methods disclosed herein comprises an Fc variant comprising more than one amino acid substitution. The extended-PK IL-2 suitable for use in the methods disclosed herein may comprise, for example, 2, 3, 4, 5, 6, 7, 8, 9, 10 or more amino acid substitutions. Preferably, the amino acid substitutions are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In some aspects, an Fc domain includes changes in the region between amino acids 234-238, including the sequence LLGGP at the beginning of the CH2 domain. In some aspects, an Fc variant alters Fc mediated effector function, particularly ADCC, and/or decrease binding avidity for Fc receptors. In some aspects, sequence changes closer to the CH2-CH3 junction, at positions such as K322 or P331 can eliminate complement mediated cytotoxicity and/or alter avidity for FcR binding. In some aspects, an Fc domain incorporates changes at residues P238 and P331, e.g., changing the wild type prolines at these positions to serine. In some aspects, alterations in the hinge region at one or more of the three hinge cysteines, to encode CCC, SCC, SSC, SCS, or SSS at these residues can also affect FcR binding and molecular homogeneity, e.g., by elimination of unpaired cysteines that may destabilize the folded protein.

Other amino acid mutations in the Fc domain are contemplated to reduce binding to the Fc gamma receptor and Fc gamma receptor subtypes. For example, mutations at positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 322, 324, 327, 329, 330, 331, 333, 334, 335, 337, 338, 340, 356, 360, 373, 376, 378, 379, 382, 388, 389, 398, 414, 416, 419, 430, 434, 435, 437, 438 or 439 of the Fc region can alter binding as described in U.S. Pat. No. 6,737,056, issued May 18, 2004, incorporated herein by reference in its entirety. This patent reported that changing Pro331 in IgG3 to Ser resulted in six fold lower affinity as compared to unmutated IgG3, indicating the involvement of Pro331 in Fc gamma RI binding. In addition, amino acid modifications at positions 234, 235, 236, and 237, 297, 318, 320 and 322 are disclosed as potentially altering receptor binding affinity in U.S. Pat. No. 5,624,821, issued Apr. 29, 1997 and incorporated herein by reference in its entirety.

Further mutations contemplated for use include, e.g., those described in U.S. Pat. App. Pub. No. 2006/0235208, published Oct. 19, 2006 and incorporated herein by reference in its entirety. This publication describes Fc variants that exhibit reduced binding to Fc gamma receptors, reduced antibody dependent cell-mediated cytotoxicity, or reduced complement dependent cytotoxicity, that comprise at least one amino acid modification in the Fc region, including 232G, 234G, 234H, 235D, 235G, 235H, 2361, 236N, 236P, 236R, 237K, 237L, 237N, 237P, 238K, 239R, 265G, 267R, 269R, 270H, 297S, 299A, 2991, 299V, 325A, 325L, 327R, 328R, 329K, 3301, 330L, 330N, 330P, 330R, and 331L (numbering is according to the EU index), as well as double mutants 236R/237K, 236R/325L, 236R/328R, 237K/325L, 237K/328R, 325L/328R, 235G/236R, 267R/269R, 234G/235G, 236R/237K/325L, 236R/325L/328R, 235G/236R/237K, and 237K/325L/328R. Other mutations contemplated for use as described in this publication include 227G, 234D, 234E, 234G, 2341, 234Y, 235D, 2351, 235S, 236S, 239D, 246H, 255Y, 258H, 260H, 2641, 267D, 267E, 268D, 268E, 272H, 2721, 272R, 281D, 282G, 283H, 284E, 293R, 295E, 304T, 324G, 3241, 327D, 327A, 328A, 328D, 328E, 328F, 3281, 328M, 328N, 328Q, 328T, 328V, 328Y, 3301, 330L, 330Y, 332D, 332E, 335D, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 297 and 298, an insertion of A between positions 297 and 298, an insertion of S between positions 297 and 298, an insertion of D between positions 297 and 298, an insertion of G between positions 326 and 327, an insertion of A between positions 326 and 327, an insertion of T between positions 326 and 327, an insertion of D between positions 326 and 327, and an insertion of E between positions 326 and 327 (numbering is according to the EU index). Additionally, mutations described in U.S. Pat. App. Pub. No. 2006/0235208 include 227G/332E, 234D/332E, 234E/332E, 234Y/332E, 2341 332E, 234G/332E, 2351/332E, 235S/332E, 235D/332E, 235E/332E, 236S/332E, 236A/332E, 236S/332D, 236A/332D, 239D/268E, 246H/332E, 255Y/332E, 258H/332E, 260H/332E, 2641 332E, 267E/332E, 267D/332E, 268D/332D, 268E/332D, 268E/332E, 268D/332E, 268E/330Y, 268D/330Y, 272R/332E, 272H/332E, 283H/332E, 284E/332E, 293R/332E, 295E/332E, 304T/332E, 3241 332E, 324G/332E, 324I/332D, 324G/332D, 327D/332E, 328A/332E, 328T/332E, 328V/332E, 3281 332E, 328F/332E, 328Y/332E, 328M/332E, 328D/332E, 328E/332E, 328N/332E, 328Q/332E, 328A/332D, 328T/332D, 328V/332D, 3281 332D, 328F/332D, 328Y/332D, 328M/332D, 328D/332D, 328E/332D, 328N/332D, 328Q/332D, 330L/332E, 330Y/332E, 3301 332E, 332D/330Y, 335D/332E, 239D/332E, 239D/332E/330Y, 239D/332E/330L, 239D/332E/330I, 239D/332E/268E, 239D/332E/268D, 239D/332E/327D, 239D/332E/284E, 239D/268E/330Y, 239D/332E/268E/330Y, 239D/332E/327A, 239D/332E/268E/327A, 239D/332E/330Y/327A, 332E/330Y/268 E/327A, 239D/332E/268E/330Y/327A, Insert G>297-298/332E, Insert A>297-298/332E, Insert S>297-298/332E, Insert D>297-298/332E, Insert G>326-327/332E, Insert A>326-327/332E, Insert T>326-327/332E, Insert D>326-327/332E, Insert E>326-327/332E, Insert G>235-236/332E, Insert A>235-236/332E, Insert S>235-236/332E, Insert T>235-236/332E, Insert N>235-236/332E, Insert D>235-236/332E, Insert V>235-236/332E, Insert L>235-236/332E, Insert G>235-236/332D, Insert A>235-236/332D, Insert S>235-236/332D, Insert T>235-236/332D, Insert N>235-236/332D, Insert D>235-236/332D, Insert V>235-236/332D, and Insert L>235-236/332D (numbering according to the EU index) are contemplated for use. The mutant L234A/L235A is described, e.g., in U.S. Pat. App. Pub. No. 2003/0108548, published Jun. 12, 2003 and incorporated herein by reference in its entirety. In embodiments, the described modifications are included either individually or in combination. In certain embodiments, the mutation is D265A in human IgG1.

In certain embodiments, the extended-PK IL-2 suitable for use in the methods disclosed herein comprises an amino acid substitution to an Fc domain which alters antigen-independent effector functions of the polypeptide, in particular the circulating half-life of the polypeptide.

In certain embodiments, the extended-PK IL-2 suitable for use in the methods disclosed herein comprises an Fc variant comprising an amino acid substitution which alters the antigen-dependent effector functions of the polypeptide, in particular ADCC or complement activation, e.g., as compared to a wild type Fc region. Such extended-PK IL-2 polypeptides exhibit decreased binding to FcR gamma when compared to wild-type polypeptides and, therefore, mediate reduced effector function. Fc variants with decreased FcR gamma binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation.

In certain embodiments, the extended-PK IL-2 exhibits altered binding to an activating FcγR (e.g. Fcγl, Fcγlla, or FcγRIIIa). In certain embodiments, the extended-PK IL-2 exhibits altered binding affinity to an inhibitory FcγR (e.g. FcγRIIb). Exemplary amino acid substitutions which altered FcR or complement binding activity are disclosed in International PCT Publication No. WO05/063815 which is incorporated by reference herein.

The extended-PK IL-2 suitable for use in the methods disclosed herein may also comprise an amino acid substitution which alters the glycosylation of the extended-PK IL-2. For example, the Fc domain of the extended-PK IL-2 may comprise an Fc domain having a mutation leading to reduced glycosylation (e.g., N- or O-linked glycosylation) or may comprise an altered glycoform of the wild-type Fc domain (e.g., a low fucose or fucose-free glycan). In certain embodiments, the extended-PK IL-2 has an amino acid substitution near or within a glycosylation motif, for example, an N-linked glycosylation motif that contains the amino acid sequence NXT or NXS. Exemplary amino acid substitutions which reduce or alter glycosylation are disclosed in WO05/018572 and US2007/0111281, the contents of which are incorporated by reference herein. In certain embodiments, the extended-PK IL-2 suitable for use in the methods disclosed herein comprises at least one Fc domain having engineered cysteine residue or analog thereof which is located at the solvent-exposed surface. In certain embodiments, the extended-PK IL-2 suitable for use in the methods disclosed herein comprise an Fc domain comprising at least one engineered free cysteine residue or analog thereof that is substantially free of disulfide bonding with a second cysteine residue. Any of the above engineered cysteine residues or analogs thereof may subsequently be conjugated to a functional domain using art-recognized techniques (e.g., conjugated with a thiol-reactive heterobifunctional linker).

In certain embodiments, the extended-PK IL-2 suitable for use in the methods disclosed herein may comprise a genetically fused Fc domain having two or more of its constituent Fc domains independently selected from the Fc domains described herein. In certain embodiments, the Fc domains are the same. In certain embodiments, at least two of the Fc domains are different. For example, the Fc domains of the extended-PK IL-2 suitable for use in the methods disclosed herein comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In certain embodiments, the Fc domains of the extended-PK IL-2 suitable for use in the methods disclosed herein may differ in sequence at one or more amino acid positions. For example, at least two of the Fc domains may differ at about 5 amino acid positions (e.g., 1, 2, 3, 4, or 5 amino acid positions), about 10 positions, about 15 positions, about 20 positions, about 30 positions, about 40 positions, or about 50 positions).

### C. PEGylation

In certain embodiments, an extended-PK IL-2 suitable for use in the methods disclosed herein includes a polyethylene glycol (PEG) domain. PEGylation is well known in the art to confer increased circulation half-life to proteins. Methods of PEGylation are well known and disclosed in, e.g., U.S. Pat. No.7,610,156, U.S. Pat. No. 7,847,062, all of which are hereby incorporated by reference.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X-0(CH₂CH₂0)ₙ-₁CH₂CH₂OH, where n is 20 to 2300 and X is H or a terminal modification, e.g., a C₁₋₄ alkyl. In certain embodiments, the PEG suitable for use in the methods disclosed herein terminates on one end with hydroxy or methoxy, i.e., X is H or CH₃ (“methoxy PEG”). PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462, both of which are hereby incorporated by reference. One form of PEGs includes two PEG side-chains (PEG2) linked via the primary amino groups of a lysine (Monfardini et al., Bioconjugate Chem 1995;6:62-9).

In certain embodiments, pegylated IL-2 is produced by site-directed pegylation, particularly by conjugation of PEG to a cysteine moiety at the N- or C-terminus. A PEG moiety may also be attached by other chemistry, including by conjugation to amines. PEG conjugation to peptides or proteins generally involves the activation of PEG and coupling of the activated PEG-intermediates directly to target proteins/peptides or to a linker, which is subsequently activated and coupled to target proteins/peptides (see Abuchowski et al., JBC 1977;252:3571 and JBC 1977;252:3582, and Harris et. al., in: Poly(ethylene glycol) Chemistry: Biotechnical and Biomedical Applications; (J. M. Harris ed.) Plenum Press: New York, 1992; Chap. 21 and 22). A variety of molecular mass forms of PEG can be selected, e.g., from about 1,000 Daltons (Da) to 100,000 Da (n is 20 to 2300), for conjugating to IL-2. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

One skilled in the art can select a suitable molecular mass for PEG, e.g., based on how the pegylated IL-2 will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 1993;10:91-114.

In certain embodiments, PEG molecules may be activated to react with amino groups on IL-2 such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)).

In certain embodiments, carbonate esters of PEG are used to form the PEG-IL-2 conjugates. N,N′-disuccinimidylcarbonate (DSC) may be used in the reaction with PEG to form active mixed PEG-succinimidyl carbonate that may be subsequently reacted with a nucleophilic group of a linker or an amino group of IL-2 (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively. Pegylation of IL-2 can be performed according to the methods of the state of the art, for example by reaction of IL-2 with electrophilically active PEGs (Shearwater Corp., USA, www.shearwatercorp.com). Preferred PEG reagents suitable for use in the methods disclosed herein are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C, et al., Bioconjugate Chem. 6 (1995) 62-69).

In certain embodiments, PEG molecules may be coupled to sulfhydryl groups on IL-2 (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. No. 6,610,281 and U.S. Pat. No. 5,766,897 describe exemplary reactive PEG species that may be coupled to sulfhydryl groups.

In certain embodiments where PEG molecules are conjugated to cysteine residues on IL-2 the cysteine residues are native to IL-2 whereas in certain embodiments, one or more cysteine residues are engineered into IL-2. Mutations may be introduced into the coding sequence of IL-2 to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein.

In certain embodiments, pegylated IL-2 comprise one or more PEG molecules covalently attached to a linker.

In certain embodiments, IL-2 is pegylated at the C-terminus. In certain embodiments, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5): 1005-1009. Monopegylation of IL-2 can also be achieved according to the general methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of IL-2 to activated PEG in the conjugation reaction can be from about 1:0.5 to 1:50, between from about 1: 1 to 1:30, or from about 1:5 to 1: 15. Various aqueous buffers can be used to catalyze the covalent addition of PEG to IL-2, or variants thereof. In certain embodiments, the pH of a buffer used is from about 7.0 to 9.0. In certain embodiments, the pH is in a slightly basic range, e.g., from about 7.5 to 8.5. Buffers having a pKa close to neutral pH range may be used, e.g., phosphate buffer.

Conventional separation and purification techniques known in the art can be used to purify PEGylated IL-2, such as size exclusion (e.g. gel filtration) and ion exchange chromatography. Products may also be separated using SDS-PAGE. Products that may be separated include mono-, di-, tri- poly- and un-pegylated IL-2 as well as free PEG. The percentage of mono-PEG conjugates can be controlled by pooling broader fractions around the elution peak to increase the percentage of mono-PEG in the composition.

In certain embodiments, PEGylated IL-2 suitable for use in the methods disclosed herein contains one, two or more PEG moieties. In certain embodiments, the PEG moiety(ies) are bound to an amino acid residue which is on the surface of the protein and/or away from the surface that contacts CD25. In certain embodiments, the combined or total molecular mass of PEG in PEG-IL-2 is from about 3,000 Da to 60,000 Da, optionally from about 10,000 Da to 36,000 Da. In certain embodiments, PEG in pegylated IL-2 is a substantially linear, straight-chain PEG.

In certain embodiments, pegylated IL-2 suitable for use in the methods disclosed herein will preferably retain at least 25%, 50%, 60%, 70%, 80%, 85%, 90%, 95% or 100% of the biological activity associated with the unmodified protein. In certain embodiments, biological activity refers to the ability to bind CD25. The serum clearance rate of PEG-modified IL-2 may be decreased by about 10%, 20%, 30%, 40%, 50%, 60%, 70%, 80%, or even 90%, relative to the clearance rate of the unmodified IL-2. PEG-modified IL-2 may have a circulation half-life (t″) which is enhanced relative to the half-life of unmodified IL-2. The half-life of PEG-IL-2, or variants thereof, may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of unmodified IL-2. In certain embodiments, the protein half-life is determined in vitro, such as in a buffered saline solution or in serum. In certain embodiments, the protein half-life is an in vivo circulation half-life, such as the half-life of the protein in the serum or other bodily fluid of an animal.

### D. Other Extended-PK Groups

In certain embodiments, the extended-PK group is a serum albumin, or fragments thereof. Methods of fusing serum albumin to proteins are disclosed in, e.g., US2010/0144599, US2007/0048282, and US2011/0020345, which are herein incorporated by reference in their entirety. In certain embodiments, the extended-PK group is human serum albumin (HSA), or variants or fragments thereof, such as those disclosed in U.S. Pat. No. 5,876,969, WO 2011/124718, WO 2013/075066, and WO 2011/0514789.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety.

In certain embodiments, the extended-PK group is transferrin, as disclosed in U.S. Pat. No. 7,176,278 and U.S. Pat. No. 8,158,579, which are herein incorporated by reference in their entirety.

In certain embodiments, the extended-PK group is a serum immunoglobulin binding protein such as those disclosed in US2007/0178082, which is herein incorporated by reference in its entirety.

In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

### E. Linkers

In certain embodiments, the extended-PK group is optionally fused to IL-2 via a linker. Linkers suitable for fusing the extended-PK group to IL-2 are well known in the art, and are disclosed in, e.g., US2010/0210511 US2010/0179094, and US2012/0094909, which are herein incorporated by reference in its entirety. Exemplary linkers include gly-ser polypeptide linkers, glycine-proline polypeptide linkers, and proline-alanine polypeptide linkers. In certain embodiments, the linker is a gly-ser polypeptide linker, i.e., a peptide that consists of glycine and serine residues.

Exemplary gly-ser polypeptide linkers comprise the amino acid sequence Ser(Gly₄Ser)n. In certain embodiments, n=1. In certain embodiments, n=2. In certain embodiments, n=3, i.e., Ser(Gly₄Ser)3. In certain embodiments, n=4, i.e., Ser(Gly₄Ser)4. In certain embodiments, n=5. In certain embodiments, n=6. In certain embodiments, n=7. In certain embodiments, n=8. In certain embodiments, n=9. In certain embodiments, n=10. Another exemplary gly-ser polypeptide linker comprises the amino acid sequence Ser(Gly₄Ser)n. In certain embodiments, n=1. In certain embodiments, n=2. In certain embodiments, n=3. In certain embodiments, n=4. In certain embodiments, n=5. certain embodiments, n=6. Another exemplary gly-ser polypeptide linker comprises (Gly₄Ser)n. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises (Gly₃Ser)n. In certain embodiments, n=1. In certain embodiments, n=2. In certain embodiments, n=3. In certain embodiments, n=4. In certain embodiments, n=5. In certain embodiments n=6.

### Tumor Associated Antigen Antibodies

Therapeutic monoclonal antibodies have been conceived as a class of pharmaceutically active agents which should allow tumor selective treatment by targeting tumor selective antigens or epitopes.

Antibodies against tumor associated antigens suitable for use in the methods disclosed herein are administered as a combinatorial therapeutic with IL-2 (e.g., extended-PK IL-2), a cancer vaccine, and optionally an immune checkpoint blocker.

Methods of producing antibodies, and antigen-binding fragments thereof, are well known in the art and are disclosed in, e.g., U.S. Pat. No. 7,247,301, U.S. Pat. No. 7,923,221, and U.S. Patent Application 2008/0138336, all of which are herein incorporated by reference in their entirety.

Therapeutic antibodies that can be used in the methods of the present invention include, but are not limited to, any of the art-recognized anti-cancer antibodies that are approved for use, in clinical trials, or in development for clinical use. In certain embodiments, more than one anti-cancer antibody can be included in the combination therapy of the present invention.

Non-limiting examples of anti-cancer antibodies include the following, without limitation: trastuzumab (HERCEPTIN™ by Genentech, South San Francisco, Calif.), which is used to treat HER-2/neu positive breast cancer or metastatic breast cancer; bevacizumab (AVASTIN™ by Genentech), which are used to treat colorectal cancer, metastatic colorectal cancer, breast cancer, metastatic breast cancer, non-small cell lung cancer, or renal cell carcinoma; rituximab (RITUXAN™ by Genentech), which is used to treat non-Hodgkin's lymphoma or chronic lymphocytic leukemia; pertuzumab (OMNITARG™ by Genentech), which is used to treat breast cancer, prostate cancer, non-small cell lung cancer, or ovarian cancer; cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer; IMC-1C11 (ImClone Systems Incorporated), which is used to treat colorectal cancer, head and neck cancer, as well as other potential cancer targets; tositumomab and tositumomab and iodine I 131 (BEXXAR XM by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy; In¹¹¹ ibirtumomab tiuxetan; Y⁹⁰ ibirtumomab tiuxetan; In111 ibirtumomab tiuxetan and Y⁹⁰ ibirtumomab tiuxetan (ZEVALIN™ by Biogen Idee, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma; EMD 7200 (EMD Pharmaceuticals, Durham, N.C.), which is used for treating non-small cell lung cancer or cervical cancer; SGN-30 (a genetically engineered monoclonal antibody targeted to CD30 antigen by Seattle Genetics, Bothell, Wash.), which is used for treating Hodgkin's lymphoma or non-Hodgkin's lymphoma; SGN-15 (a genetically engineered monoclonal antibody targeted to a Lewisy-related antigen that is conjugated to doxorubicin by Seattle Genetics), which is used for treating non-small cell lung cancer; SGN-33 (a humanized antibody targeted to CD33 antigen by Seattle Genetics), which is used for treating acute myeloid leukemia (AML) and myelodysplasia syndromes (MDS); SGN-40 (a humanized monoclonal antibody targeted to CD40 antigen by Seattle Genetics), which is used for treating multiple myeloma or non-Hodgkin's lymphoma; SGN-35 (a genetically engineered monoclonal antibody targeted to a CD30 antigen that is conjugated to auristatin E by Seattle Genetics), which is used for treating non-Hodgkin's lymphoma; SGN-70 (a humanized antibody targeted to CD70 antigen by Seattle Genetics), which is used for treating renal cancer and nasopharyngeal carcinoma; SGN-75 (a conjugate comprised of the SGN70 antibody and an Auristatin derivative by Seattle Genetics); and SGN-17/19 (a fusion protein containing antibody and enzyme conjugated to melphalan prodrug by Seattle Genetics), which is used for treating melanoma or metastatic melanoma.

It should be understood that the therapeutic antibodies to be used in the methods of the present invention are not limited to those described supra. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chromic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies suitable for use in the methods disclosed herein can also target molecules expressed by immune cells, such as, but not limited to, 0×86 which targets OX40 and increases antigen-specific CD8+ T cells at tumor sites and enhances tumor rejection; BMS-663513 which targets CD137 and causes regression of established tumors, as well as the expansion and maintenance of CD8+ T cells, and daclizumab (ZENAPAXTM) which targets CD25 and causes transient depletion of CD4+CD25+FOXP3+Tregs and enhances tumor regression and increases the number of effector T cells. A more detailed discussion of these antibodies can be found in, e.g., Weiner et al., Nature Rev. Immunol 2010;10:317-27.

Other therapeutic antibodies can be identified that target tumor antigens (e.g., tumor antigens associated with different types of cancers, such as carcinomas, sarcomas, myelomas, leukemias, lymphomas, and combinations thereof). For example, the following tumor antigens can be targeted by therapeutic antibodies in the methods disclosed herein.

The tumor antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In certain embodiments, the tumor antigen is a lymphoma antigen (e.g., non-Hodgkin's lymphoma or Hodgkin's lymphoma), a B-cell lymphoma cancer antigen, a leukemia antigen, a myeloma (e.g., multiple myeloma or plasma cell myeloma) antigen, an acute lymphoblastic leukemia antigen, a chronic myeloid leukemia antigen, or an acute myelogenous leukemia antigen. It should be understood that the described tumor antigens are only exemplary and that any tumor antigen can be targeted for use in the methods disclosed herein.

In certain embodiments, the tumor antigen is a mucin-1 protein or peptide (MUC-1) that is found on most or all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293).

In certain embodiments, the tumor antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653(1):25-40; Sharkey et al., Cancer Res. (2004) 64(5):1595-1599).

In certain embodiments, the tumor antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™. Accession No. AAA58637. Based on these sequences, one skilled in the art could develop HER-2/neu antigens using known assays to find appropriate epitopes that generate an effective immune response. Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9₇₅₄]; and the like.

In certain embodiments, the tumor antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR⁺ cancers. Exemplary EGFR⁺ cancers include lung cancer, head and neck cancer, colon cancer, colorectal cancer, breast cancer, prostate cancer, gastric cancer, ovarian cancer, brain cancer and bladder cancer.

In certain embodiments, the tumor antigen is a vascular endothelial growth factor receptor (VEGFR) antigen. VEGFR is considered to be a regulator of cancer-induced angiogenesis. Cancers with cells that overexpress VEGFR are called VEGFR⁺ cancers. Exemplary VEGFR⁺ cancers include breast cancer, lung cancer, small cell lung cancer, colon cancer, colorectal cancer, renal cancer, leukemia, and lymphocytic leukemia.

In certain embodiments, the tumor antigen is prostate-specific antigen (PSA) and/or prostate-specific membrane antigen (PSMA) that are prevalently expressed in androgen-independent prostate cancers.

In certain embodiments, the tumor antigen is Glycoprotein 100 (gp 100), a tumor-specific antigen associated with melanoma.

In certain embodiments, the tumor antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

In certain embodiments, the tumor antigen is carbohydrate antigen 10.9 (CA 19.9). CA 19.9 is an oligosaccharide related to the Lewis A blood group substance and is associated with colorectal cancers.

In certain embodiments, the tumor antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMe117; pMe117/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

In certain embodiments, the tumor antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In certain embodiments, the tumor antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

Further tumor antigens are well known in the art and include, for example, a glioma-associated antigen, carcinoembryonic antigen (CEA), 13-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxy esterase, mut hsp70-2, M-CSF, prostase, prostate-specific antigen (PSA), PAP, NY-ESO-1, LAGE-1a, p53, tyrosinase, prostein, PSMA, ras, Her2/neu, TRP-1, TRP-2, TAG-72, KSA, CA-125, PSA, BRCI, BRC-II, bcr-ab1, pax3-fkhr, ews-fli-1, survivin and telomerase, prostate-carcinoma tumor antigen-1 (PCTA-1), MAGE, GAGE, GP-100, MUC-1, MUC-2, ELF2M, neutrophil elastase, ephrinB2, CD22, insulin growth factor (IGF)-I, IGF-II, IGF-I receptor, and mesothelin,

In certain embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The tumor antigen may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-1), Pmel 17, tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 1 80erbB-3, c-met, nm-23H 1, PSA, TAG-72, CA 19-9, CA 72-4, CAM 17.1, NuMa, K-ras, beta-Catenin, CDK4, Mum-1, p 15, p 16, 43-9F, 5T4(791Tgp72₁ alpha-fetoprotem, beta-HCG, BCA225, BTAA, CA 125, CA 15-3\CA 27.29\BCAA, CA 195, CA 242, CA-50, CAM43, CD68\ I, CO-029, FGF-5, G250, Ga733VEpCAM, HTgp-175, M344, MA-50, MG7-Ag, MOV 18, NB/70K, NY-CO-1, RCAS 1, SDCCAG16, TA-90\Mac-2 binding protein, Acyclophilin C-associated protein, TAAL6, TAG72, TLP, and TPS.

In certain embodiments, the tumor-associated antigen is determined by sequencing a patient's tumor cells and identifying mutated proteins only found in the tumor. These antigens are referred to as “neoantigens.” Once a neoantigen has been identified, therapeutic antibodies can be produced against it and used in the methods described herein.

The therapeutic antibody can be a fragment of an antibody; a complex comprising an antibody; or a conjugate comprising an antibody. The antibody can optionally be chimeric or humanized or fully human.

### Cancer Vaccine

A. Overview

In certain embodiments, cancer vaccines are used in addition to the other therapeutic agents described herein (e.g., extended-PK IL-2, therapeutic antibody, and optional immune checkpoint blocker). In certain embodiments, the cancer vaccine stimulates a specific immune response against a specific target, such as a tumor-associated antigen.

In certain embodiments, the cancer vaccine will include viral, bacterial or yeast vectors to deliver recombinant genes to antigen presenting cells (APCs). In certain embodiments the cancer vaccine uses autologous or allogeneic tumor cells. In certain embodiments, these tumor cells may be modified for expression of MHC, costimulatory molecules, or cytokines.

In certain embodiments, the tumor-associated antigen is determined by sequencing a patient's tumor cells and identifying mutated proteins only found in the tumor. These antigens are referred to as “neoantigens.” Once a neoantigen has been identified, it can be used as the antigen for a vaccine or for developing monoclonal antibodies specifically reactive with the neoantigen.

In certain embodiments, the vaccine includes irradiated tumor cells transduced with cytokines such as GM-CSF or loaded with adjuvant compounds, such as the GM-CSF-secreting tumor cell vaccine GVAX (Immunological Reviews, 222(1): 287-298, 2008). In certain embodiments the vaccine includes one or more tumor-associated antigens in the form of an immunogenic composition, optionally in combination with an adjuvant. For example, vaccination against HPV-16 oncoproteins resulted in positive clinical outcomes for vulvar intraepithelial neoplasia (The New England Journal of Medicine, 361(19), 1838-1847. 2012). Also, multipeptide immune response to cancer vaccine IMA901 after single-dose cyclophosphamide associates with longer patient survival (Nature Medicine., 18(8): 1254-61, 2012). Alternatively, a DNA based approach can be used to immunize a patient with one or more tumor-associated antigens. Improved tumor immunity is observed using a DNA vaccine in combination with an anti-tyrosinase related protein-1 monoclonal antibody in murine melanoma (Cancer Research, 68(23), 9884-9891, 2008).

Other vaccine approaches utilize patient immune cells, such as dendritic cells which can be cultured with a tumor-associated antigen to produce antigen presenting cells that will stimulate the immune system and target the antigen of interest. A current FDA approved cancer treatment vaccine using this approach is Provenge® (Dendreon), approved for use in some men with metastatic prostate cancer. This vaccine stimulates an immune response to prostatic acid phosphatase (PAP), an antigen found on most prostate cancer cells. The vaccine is created by isolating a specific patient's immune cells and culturing dendritic cells with PAP to produce antigen presenting cells that will stimulate the immune system and target PAP. These and other cancer vaccines can be used in combination with other treatments as described herein.

B. Amphiphile Vaccines

In certain embodiments, an amphiphile vaccine, as described in US 2013/0295129, herein incorporated -by reference, is used in the methods disclosed herein. An amphiphile vaccine combines an albumin-binding lipid and a peptide antigen or molecular adjuvant to efficiently target the peptide or adjuvant to lymph nodes in vivo. Lipid conjugates bind to endogenous albumin, which targets them to lymphatics and draining lymph nodes where they accumulate due to the filtering of albumin by antigen presenting cells. When the lipid conjugate includes an antigenic peptide or molecular adjuvant, the conjugates induce or enhance a robust immune response.

Lymph node-targeting conjugates typically include three domains: a highly lipophilic, albumin-binding domain (e.g., an albumin-binding lipid), a cargo such as a molecular adjuvant or a peptide antigen, and a polar block linker, which promotes solubility of the conjugate and reduces the ability of the lipid to insert into cellular plasma membranes. Accordingly, in certain embodiments, the general structure of the conjugate is L-P-C, where “L” is an albumin-binding lipid, “P” is a polar block, and “C” is a cargo such as a molecular adjuvant or a polypeptide. In some embodiments, the cargo itself can also serve as the polar block domain, and a separate polar block domain is not required. Therefore, in certain embodiments the conjugate has only two domains: an albumin-binding lipid and a cargo.

The cargo of the conjugates suitable for use in the methods disclosed herein is typically a molecular adjuvant such as an immunostimulatory oligonucleotide, or a peptide antigen. However, the cargo can also be other oligonucleotides, peptides, Toll-like receptor agonists or other immunomodulatory compounds, dyes, MRI contrast agents, fluorophores or small molecule drugs that require efficient trafficking to the lymph nodes.

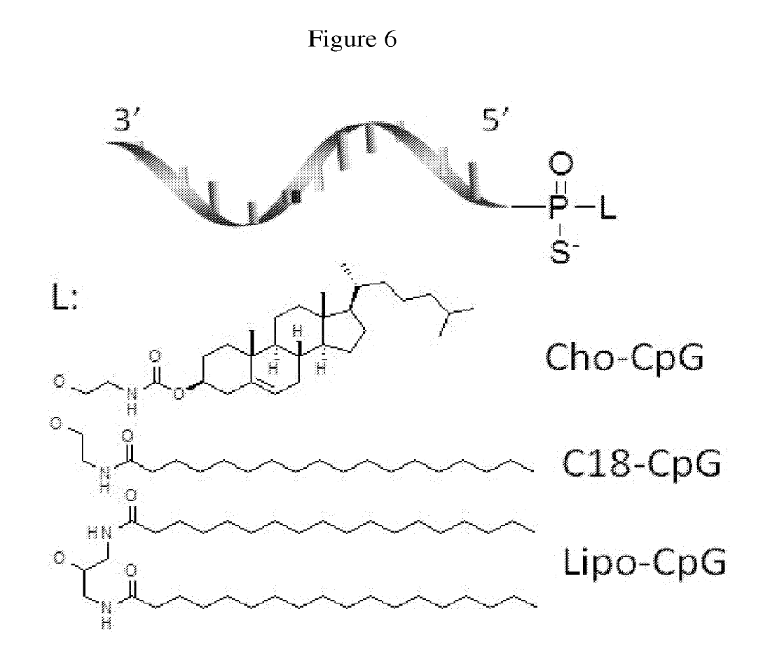

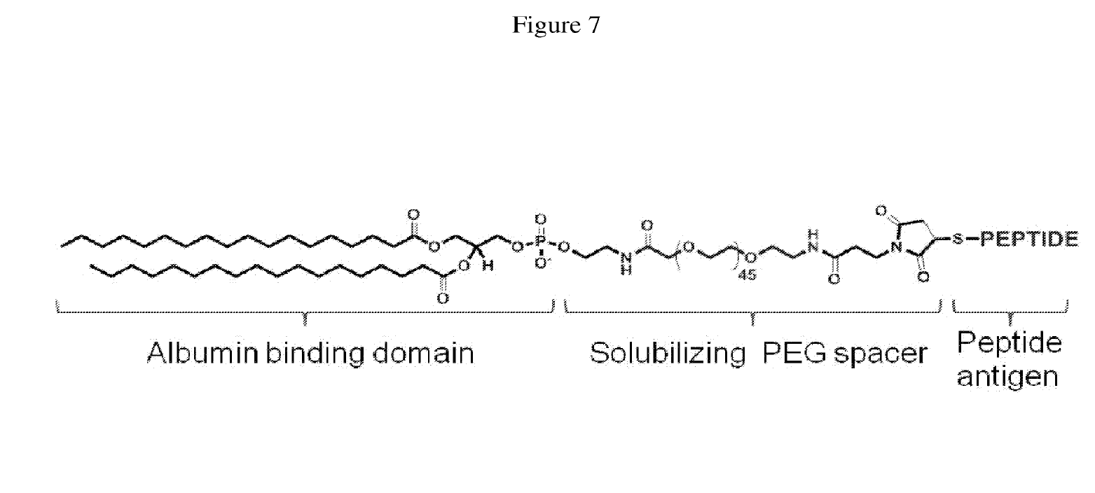

In certain embodiments, a lipid-oligonucleotide conjugates includes an immunostimulatory oligonucleotide which is conjugated directly to a lipid, or is linked to a linker which is conjugated to a lipid. A schematic representation of an exemplary lipid-oligonucleotide conjugate is shown in FIG. 6. Other embodiments are directed to lipid-peptide conjugates which include an antigenic peptide conjugated directly to a lipid, or is linked to a linker which is conjugated to a lipid. A schematic representation of an exemplary lipid-peptide conjugate is shown in FIG. 7.

(i) Lipids

The lipid conjugates typically include a hydrophobic lipid. The lipid can be linear, branched, or cyclic. The lipid is preferably at least 17 to 18 carbons in length, but may be shorter if it shows good albumin binding and adequate targeting to the lymph nodes. Lymph node-targeting conjugates include lipid-oligonucleotide conjugates and lipid-peptide conjugates that can be trafficked from the site of delivery through the lymph to the lymph node. In certain embodiments, the activity relies, in-part, on the ability of the conjugate to associate with albumin in the blood of the subject. Therefore, lymph node-targeted conjugates typically include a lipid that can bind to albumin under physiological conditions. Lipids suitable for targeting the lymph node can be selected based on the ability of the lipid or a lipid conjugate including the lipid to bind to albumin. Suitable methods for testing the ability of the lipid or lipid conjugate to bind to albumin are known in the art.

For example, in certain embodiments, a plurality of lipid conjugates is allowed to spontaneously form micelles in aqueous solution. The micelles are incubated with albumin, or a solution including albumin such as Fetal Bovine Serum (FBS). Samples can be analyzed, for example, by ELISA, size exclusion chromatography or other methods to determine if binding has occurred. Lipid conjugates can be selected as lymph node-targeting conjugates if in the presence of albumin, or a solution including albumin such as Fetal Bovine Serum (FBS), the micelles dissociate and the lipid conjugates bind to albumin as discussed above.

Examples of preferred lipids for use in lymph node targeting lipid conjugates include, but are not limited to fatty acids with aliphatic tails of 8-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids; Lipid A or combinations thereof.

In certain embodiments, the lipid is a diacyl lipid or two-tailed lipid. In some embodiments, the tails in the diacyl lipid contain from about 8 to about 30 carbons and can be saturated, unsaturated, or combinations thereof. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

Preferably, lymph node-targeting conjugates include a lipid that is 8 or more carbon units in length. It is believed that increasing the number of lipid units can reduce insertion of the lipid into plasma membrane of cells, allowing the lipid conjugate to remain free to bind albumin and traffic to the lymph node.

For example, the lipid can be a diacyl lipid composed of two C18 hydrocarbon tails. In certain embodiments, the lipid for use in preparing lymph node targeting lipid conjugates is not a single chain hydrocarbon (e.g., C18), or cholesterol. Cholesterol conjugation has been explored to enhance the immunomodulation of molecular adjuvants such as CpG and immunogenicity of peptides, but cholesterol conjugates, which associate well with lipoproteins but poorly with albumin, show poor lymph node targeting and low immunogenicity in vaccines compared to optimal albumin-binding conjugates.

(ii) Molecular Adjuvants

In certain embodiments, lipid-oligonucleotide conjugates are used in the vaccine. The oligonucleotide conjugates typically contain an immunostimulatory oligonucleotide.

In certain embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone. In certain embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNa) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

According to some embodiments, a lipophilic-CpG oligonucleotide conjugate is used to enhance an immune response to a peptide antigen. The lipophilic-CpG oligonucleotide is represented by the following, wherein “L” is a lipophilic compound, such as diacyl lipid, “Gₙ” is a guanine repeat linker and “n” represents 1, 2, 3, 4, or 5.

5′-L-GₙTCCATGACGTTCCTGACGTT-3′

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDAS), which are best known as RNA-sensing receptors in the cytosol. Therefore, in certain embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In certain embodiments, the oligonucleotide cargo includes two or more immunostimulatory sequences.

The oligonucleotide can be between 2-100 nucleotide bases in length, including for example, 5 nucleotide bases in length, 10 nucleotide bases in length, 15 nucleotide bases in length, 20 nucleotide bases in length, 25 nucleotide bases in length, 30 nucleotide bases in length, 35 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

The 3′ end or the 5′ end of the oligonucleotides can be conjugated to the polar block or the lipid. In certain embodiments the 5′ end of the oligonucleotide is linked to the polar block or the lipid.

The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds. In certain embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In certain embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In certain embodiments, the analogs have a substantially uncharged, phosphorus containing backbone.

(iii) Peptide Antigens

The peptide conjugates suitable for use in the methods disclosed herein typically include an antigenic protein or polypeptide, such as a tumor-associated antigen or portion thereof.

The peptide can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide can be greater than 50 amino acids. In some embodiments, the peptide can be >100 amino acids. A protein/peptide can be linear, branched or cyclic. The peptide can include D amino acids, L amino acids, or a combination thereof. The peptide or protein can be conjugated to the polar block or lipid at the N-terminus or the C-terminus of the peptide or protein.

The protein or polypeptide can be any protein or peptide that can induce or increase the ability of the immune system to develop antibodies and T-cell responses to the protein or peptide. A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be, but is not limited to, TRP-1, TRP-2, MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-Al2, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2. Additional cancer antigens include the tumor antigens described herein.

Suitable antigens are known in the art and are available from commercial government and scientific sources. In certain embodiments, the antigens are whole inactivated or irradiated tumor cells. The antigens may be purified or partially purified polypeptides derived from tumors. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

In certain embodiments, antigens may be provided as single antigens or may be provided in combination. Antigens may also be provided as complex mixtures of polypeptides or nucleic acids.

(iv) Polar Block/Linker

For the conjugate to be trafficked efficiently to the lymph node, the conjugate should remain soluble. Therefore, a polar block linker can be included between the cargo and the lipid to increase solubility of the conjugate. The polar block reduces or prevents the ability of the lipid to insert into the plasma membrane of cells, such as cells in the tissue adjacent to the injection site. The polar block can also reduce or prevent the ability of cargo, such as synthetic oligonucleotides containing a PS backbone, from non-specifically associating with extracellular matrix proteins at the site of administration. The polar block increases the solubility of the conjugate without preventing its ability to bind to albumin. It is believed that this combination of characteristics allows the conjugate to bind to albumin present in the serum or interstitial fluid, and remain in circulation until the albumin is trafficked to, and retained in a lymph node. The length and composition of the polar block can be adjusted based on the lipid and cargo selected. For example, for oligonucleotide conjugates, the oligonucleotide itself may be polar enough to insure solubility of the conjugate, for example, oligonucleotides that are 10, 15, 20 or more nucleotides in length. Therefore, in certain embodiments, no additional polar block linker is required. However, depending on the amino acid sequence, some lipidated peptides can be essentially insoluble. In these cases, it can be desirable to include a polar block that mimics the effect of a polar oligonucleotide.

A polar block can be used as part of any of lipid conjugates suitable for use in the methods disclosed herein, for example, lipid-oligonucleotide conjugates and lipid-peptide conjugates, which reduce cell membrane insertion/preferential portioning ont albumin. Suitable polar blocks include, but are not limited to, oligonucleotides such as those discussed above, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof polysaccharides, including but not limited to, dextran (MW: 1,000 Da to 2,000,000 Da), or combinations thereof.

The hydrophobic lipid and the linker/cargo are covalently linked. The covalent bond may be a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

a. Ethylene Glycol Linkers

In certain embodiments, the polar block is one or more ethylene glycol (EG) units, more preferably two or more EG units (i.e., polyethylene glycol (PEG)). For example, in certain embodiments, a peptide conjugate includes a protein or peptide (e.g., peptide antigen) and a hydrophobic lipid linked by a polyethylene glycol (PEG) molecule or a derivative or analog thereof.

In certain embodiments, protein conjugates suitable for use in the methods disclosed herein contain protein antigen linked to PEG which is in turn linked to a hydrophobic lipid, or lipid-Gn-ON conjugates, either covalently or via formation of protein-oligo conjugates that hybridize to oligo micelles. The precise number of EG units depends on the lipid and the cargo, however, typically, a polar block can have between about 1 and about 100, between about 20 and about 80, between about 30 and about 70, or between about 40 and about 60 EG units. In certain embodiments, the polar block has between about 45 and 55 EG, units. For example, in certain embodiments, the polar block has 48 EG units.

b. Oligonucleotide Linkers

As discussed above, in certain embodiments, the polar block is an oligonucleotide. The polar block linker can have any sequence, for example, the sequence of the oligonucleotide can be a random sequence, or a sequence specifically chosen for its molecular or biochemical properties (e.g., highly polar). In certain embodiments, the polar block linker includes one or more series of consecutive adenine (A), cytosine (C), guanine (G), thymine (T), uracil (U), or analog thereof. In certain embodiments, the polar block linker consists of a series of consecutive adenine (A), cytosine (C), guanine (G), thymine (T), uracil (U), or analog thereof.

In certain embodiments, the linker is one or more guanines, for example between 1-10 guanines. It has been discovered that altering the number of guanines between a cargo such as a CpG oligonucleotide, and a lipid tail controls micelle stability in the presence of serum proteins. Therefore, the number of guanines in the linker can be selected based on the desired affinity of the conjugate for serum proteins such as albumin. When the cargo is a CpG immunostimulatory oligonucleotide and the lipid tail is a diacyl lipid, the number of guanines affects the ability of micelles formed in aqueous solution to dissociate in the presence of serum: 20% of the non-stabilized micelles (lipo-G₀T₁₀-CG) were intact, while the remaining 80% were disrupted and bonded with FBS components. In the presence of guanines, the percentage of intact micelles increased from 36% (lipo-G₂T₈-CG) to 73% (lipo-G₄T₆-CG), and finally reached 90% (lipo-G₆T₄-CG). Increasing the number of guanines to eight (lipo-G₈T₂-CG) and ten (lipo-G₁₀T₀-CG) did not further enhance micelle stability.

Therefore, in certain embodiments, the linker in a lymph node-targeting conjugate suitable for use in the methods disclosed herein can include 0, 1, or 2 guanines. As discussed in more detail below, linkers that include 3 or more consecutive guanines can be used to form micelle-stabilizing conjugates with properties that are suitable for use in the methods disclosed herein.

C. Immunogenic Compositions

The conjugates suitable for use in the methods disclosed herein can be used in immunogenic compositions or as components in vaccines. Typically, immunogenic compositions disclosed herein include an adjuvant, an antigen, or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. When administered in combination, the adjuvant can be a lipid conjugate, the antigen can be a lipid conjugate, or the adjuvant and the antigen can both be lipid conjugates.

An immunogenic composition suitable for use in the methods disclosed herein can include a lipid conjugate that is an antigen such as an antigenic polypeptide-lipid conjugate, administered alone, or in combination with an adjuvant. The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include, without limitation, double-stranded RNA. Adjuvants that act through TLR4 include, without limitation, derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include, without limitation, flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

### Immune Checkpoint Blocker

In certain embodiments, immune checkpoint blockers are used in combination with other therapeutic agents described herein (e.g., extended-PK IL-2, therapeutic antibody, and cancer vaccine). T cell activation and effector functions are balanced by co-stimulatory and inhibitory signals, referred to as “immune checkpoints.” Inhibitory ligands and receptors that regulate T cell effector functions are overexpressed on tumor cells. Subsequently, agonists of co-stimulatory receptors or antagonists of inhibitory signals, result in the amplification of antigen-specific T cell responses. In contrast to therapeutic antibodies which target tumor cells directly, immune checkpoint blocker enhances endogenous anti-tumor activity. In certain embodiments, the immune checkpoint blocker suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardo11, D., Nature. 12: 252-264, 2012.

Disclosed herein are methods for treating a subject afflicted with diseases such as cancer, which methods comprise administering to the subject a composition comprising a therapeutically effective amount of an immune checkpoint blocker, IL-2 (e.g., extended-PK IL-2), and a therapeutic antibody. In certain embodiments, the immune checkpoint blocker is an antibody or an antigen-binding portion thereof, that disrupts or inhibits signaling from an inhibitory immunoregulator. In certain embodiments, the immune checkpoint blocker is a small molecule that disrupts or inhibits signaling from an inhibitory immunoregulator.

In certain embodiments, the inhibitory immunoregulator (immune checkpoint blocker) is a component of the PD-1/PD-L1 signaling pathway. Accordingly, certain embodiments of the invention provide methods for immunotherapy of a subject afflicted with cancer, which methods comprise administering to the subject a therapeutically effective amount of an antibody or an antigen-binding portion thereof that disrupts the interaction between the PD-1 receptor and its ligand, PD-L1. Antibodies known in the art which bind to PD-1 and disrupt the interaction between the PD-1 and its ligand, PD-L1, and stimulates an anti-tumor immune response, are suitable for use in the methods disclosed herein. In certain embodiments, the antibody or antigen-binding portion thereof binds specifically to PD-1. For example, antibodies that target PD-1 and are in clinical trials include, e.g., nivolumab (BMS-936558, Bristol-Myers Squibb) and pembrolizumab (lambrolizumab, MK03475, Merck). Other suitable antibodies for use in the methods disclosed herein are anti-PD-1 antibodies disclosed in U.S. Pat. No. 8,008,449, herein incorporated by reference. In certain embodiments, the antibody or antigen-binding portion thereof binds specifically to PD-L1 and inhibits its interaction with PD-1, thereby increasing immune activity. Antibodies known in the art which bind to PD-L1 and disrupt the interaction between the PD-1 and PD-L1, and stimulates an anti-tumor immune response, are suitable for use in the methods disclosed herein. For example, antibodies that target PD-L1 and are in clinical trials, include BMS-936559 (Bristol-Myers Squibb) and MPDL3280A (Genetech). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the methods disclosed herein.

In certain embodiments, the inhibitory immunoregulator is a component of the CTLA4 signaling pathway. Accordingly, certain embodiments of the invention provide methods for immunotherapy of a subject afflicted with cancer, which methods comprise administering to the subject a therapeutically effective amount of an antibody or an antigen-binding portion thereof that targets CTLA4 and disrupts its interaction with CD80 and CD86. Exemplary antibodies that target CTLA4 include ipilimumab (MDX-010, MDX-101, Bristol-Myers Squibb), which is FDA approved, and tremelimumab (ticilimumab, CP-675, 206, Pfizer), currently undergoing human trials. Other suitable antibodies that target CTLA4 are disclosed in WO 2012/120125, U.S. Pat. No. 6,984720, U.S. Pat. No. 6,682,7368, and U.S. Patent Applications 2002/0039581, 2002/0086014, and 2005/0201994, herein incorporated by reference. It will be understood by one of ordinary skill that any antibody which binds to CTLA4, disrupts its interaction with CD80 and CD86, and stimulates an anti-tumor immune response, is suitable for use in the methods disclosed herein.

In certain embodiments, the inhibitory immunoregulator is a component of the LAG3 (lymphocyte activation gene 3) signaling pathway. Accordingly, certain embodiments of the invention provide methods for immunotherapy of a subject afflicted with cancer, which methods comprise administering to the subject a therapeutically effective amount of an antibody or an antigen-binding portion thereof that targets LAG3 and disrupts its interaction with MHC class II molecules. An exemplary antibody that targets LAG3 is IMP321 (Immutep), currently undergoing human trials. Other suitable antibodies that target LAG3 are disclosed in U.S. Patent Application 2011/0150892, herein incorporated by reference. It will be understood by one of ordinary skill that any antibody which binds to LAG3, disrupts its interaction with MHC class II molecules, and stimulates an anti-tumor immune response, is suitable for use in the methods disclosed herein.

In certain embodiments, the inhibitory immunoregulator is a component of the B7 family signaling pathway. In certain embodiments, the B7 family members are B7-H3 and B7-H4. Accordingly, certain embodiments of the invention provide methods for immunotherapy of a subject afflicted with cancer, which methods comprise administering to the subject a therapeutically effective amount of an antibody or an antigen-binding portion thereof that targets B7-H3 or H4. The B7 family does not have any defined receptors but these ligands are upregulated on tumor cells or tumor-infiltrating cells. Preclinical mouse models have shown that blockade of these ligands can enhance anti-tumor immunity. An exemplary antibody that targets B7-H3 is MGA271 (Macrogenics), currently undergoing human trials. Other suitable antibodies that target LAG3 are disclosed in U.S. Patent Application 2013/0149236, herein incorporated by reference. It will be understood by one of ordinary skill that any antibody which binds to B7-H3 or H4, and stimulates an anti-tumor immune response, is suitable for use in the methods disclosed herein.

In certain embodiments, the inhibitory immunoregulator is a component of the TIM3 (T cell membrane protein 3) signaling pathway. Accordingly, certain embodiments of the invention provide methods for immunotherapy of a subject afflicted with cancer, which methods comprise administering to the subject a therapeutically effective amount of an antibody or an antigen-binding portion thereof that targets LAG3 and disrupts its interaction with galectin 9. Suitable antibodies that target TIM3 are disclosed in U.S. Patent Application 2013/0022623, herein incorporated by reference. It will be understood by one of ordinary skill that any antibody which binds to TIM3, disrupts its interaction with galectin 9, and stimulates an anti-tumor immune response, is suitable for use in the methods disclosed herein.

It should be understood that antibodies targeting immune checkpoints suitable for use in the methods disclosed herein are not limited to those described supra. Moreover, it will be understood by one of ordinary skill in the art that other immune checkpoint targets can also be targeted by antagonists or antibodies in the methods described herein, provided that the targeting results in the stimulation of an anti-tumor immune response as reflected in, e.g., an increase in T cell proliferation, enhanced T cell activation, and/or increased cytokine production (e.g., IFN-γ, IL-2).

### Alternatives to Immune Checkpoint Blockers

In certain embodiments, an antagonist of vascular endothelial growth factor (VEGF) is used in place of an immune checkpoint blocker. VEGF has recently been demonstrated to play a role in immune suppression (Liang, W.-C. et al. J. Biol. Chem. (2006) Vol 281: 951-961; Voron, T. et al. Front Oncol (2014) Vol. 4: Article 70; Terme, M. et al., Clin Dev Immunol (2012) Vol. 2012: Article ID 492920; Kandalaft, E. et al., Curr Top Microbiol Immunol (2011) Vol 344: 129-48), therefore blocking its activity enhance the immune response, similar to that of an immune checkpoint blocker. A “VEGF antagonist” refers to a molecule capable of neutralizing, blocking, inhibiting, abrogating, reducing or interfering with VEGF activities including its binding to one or more VEGF receptors. Non-limiting examples of VEGF antagonists include anti-VEGF antibodies and antigen-binding fragments thereof, receptor molecules and derivatives which bind specifically to VEGF thereby sequestering its binding to one or more receptors (e.g., a VEGF receptor), anti-VEGF receptor antibodies, VEGF receptor antagonists such as small molecule inhibitors of the VEGFR tyrosine kinases, or a dominant negative VEGF.

In certain embodiments, the VEGF antagonist is an antibody. An “anti-VEGF antibody” is an antibody that binds to VEGF with sufficient affinity and specificity. Non-limiting examples of anti-VEGF antibodies are described in U.S. Pat. Nos. 6,884,879, 7,060,269, 6,582,959, 6,703,030, 6,054,297, US Patent Application Nos. 2006009360, 20050186208, 20030206899, 20030190317, 20030203409, 20050112126, and PCT Publication Nos. WO 98/45332, 96/30046, 94/10202, 05/044853, 13/181452. The contents of these patents and patent applications are herein incorporated by reference. In certain embodiments the VEGF antibody is bevacizumab (Avastin ® Genentech/Roche) or ranibizumab (Lucentis® Genentech/Roche).

In certain embodiments, the VEGF antagonist binds to the VEGF receptor. VEGF receptors, or fragments thereof, that specifically bind to VEGF can be used to bind to and sequester the VEGF protein, thereby preventing it from activating downstream signaling. In certain embodiments, the VEGF receptor, or VEGF binding fragment thereof, is a soluble VEGF receptor, such as sFlt-1. The soluble form of the receptor exerts an inhibitory effect on the biological activity of VEGF by binding to VEGF, thereby preventing it from binding to its natural receptors present on the surface of target cells. Non-limiting examples of VEGF antagonists which bind the VEGF receptor are disclosed in PCT Application Nos. 97/44453, 05/000895 and U.S. Patent Application No. 20140057851.

### Engineered Fusion Molecules

Also provided herein are engineered molecules that comprise two or more of IL-2, a therapeutic antibody (e.g., an anti-tumor antibody) or antibody fragment described herein, a tumor antigen peptide (e.g., Trp1, Trp2) described herein, and a CpG oligonucleotide. Such engineered molecules can effectively reduce the number of components to be administered to a subject (e.g., a cancer patient) in the methods described herein. In some embodiments, the therapeutic antibody or antibody fragment serves as the scaffold for conjugation with other components (e.g., IL-2, tumor antigen, and/or CpG oligonucleotide).

Accordingly, in certain embodiments, the engineered molecule comprises IL-2 and a therapeutic antibody or antibody fragment. In some embodiments, the engineered molecule comprises a tumor antigen peptide and a therapeutic antibody or antibody fragment. The tumor antigen component can be used to augment the natural delivery of antigenic material from tumor cells killed by innate immune effector mechanisms. In other embodiments, the engineered molecule comprises a CpG oligonucleotide and a therapeutic antibody or antibody fragment. In yet other embodiments, the engineered molecule comprises IL-2, a therapeutic antibody or antibody fragment, and a CpG oligonucleotide. In further embodiments, the engineered molecule comprises IL-2, a therapeutic antibody or antibody fragment, a tumor antigen peptide, and a CpG oligonucleotide.

In certain embodiments, the engineered protein further comprises an immune checkpoint blocker. In some embodiments, the immune checkpoint blocker is an antibody. In a particular embodiment, the antibody for use in the engineered protein is a bispecific antibody, wherein one component is a therapeutic antibody and the other component is an antibody that binds to an immune checkpoint blocker. Methods for generating bispecific antibodies are known in the art.

Accordingly, in certain embodiments, the engineered molecule comprises IL-2 and a bispecific antibody which binds to a therapeutic target and an immune checkpoint blocker. In other embodiments, the engineered molecule comprises a tumor antigen and a bispecific antibody which binds to a therapeutic target and an immune checkpoint blocker. In yet other embodiments, the engineered molecule comprises a CpG oligonucleotide and a bispecific antibody which binds to a therapeutic target and an immune checkpoint blocker. In further embodiments, the engineered molecule comprises IL-2, a tumor antigen, and a bispecific antibody which binds to a therapeutic target and an immune checkpoint blocker. In further embodiments, the engineered molecule comprises IL-2, a CpG oligonucleotide, and a bispecific antibody which binds to a therapeutic target and an immune checkpoint blocker. In additional embodiments, the engineered molecule comprises IL-2, a tumor antigen, a CpG oligonucleotide, and a bispecific antibody which binds to a therapeutic target and an immune checkpoint blocker.

In certain embodiments, the IL-2 component for use in the engineered protein is an IL-2 lacking a pharmacokinetic moiety (i.e., a non-extended PK IL-2). In other embodiments, the IL-2 comprises a pharmacokinetic moiety (an extended-PK IL-2).

In certain embodiments, the components of the engineered molecule are conjugated to the antibody or bispecific antibody with or without a linker. Suitable linkers for conjugation are described herein and extensively described in the art.

Regions to which polypeptide-based components (e.g., tumor antigen and IL-2) of the engineered molecule can be fused, with or without a linker, to the antibody are generally known in the art, and include, for example, the C-terminus of the antibody heavy chain and the C-terminus of the antibody light chain. In certain embodiments, CpG oligonucleotides (as a cancer vaccine adjuvant) are site-specifically conjugated to artificially-induced single cysteine thiols in the antibody. In other embodiments, CpG oligonucleotides can be randomly conjugated to the therapeutic antibody or antibody fragment, as described in Yang et al. (Mol Ther 2013;21:91-100) and Schettini et al. (Cancer Immunol Immunother 2012;61:2055-65).

In certain embodiments, components of the engineered molecule do not interfere with the function of the other components. By way of example, when the engineered protein comprises a therapeutic antibody and IL-2, the IL-2 will be fused to the therapeutic antibody in a manner such that the antibody retains its antigen-binding function, and IL-2 retains the ability to interact with its receptor. Similarly, when the engineered protein comprises a therapeutic antibody and tumor antigen, the tumor antigen (e.g., a polypeptide from Trpl or Trp2) retains the ability to stimulate a specific response against the antigen, and the antibody retains its antigen-binding function. The methods described herein, e.g., in the Examples, can be used to determine whether components of the engineered protein retain their respective functions.

### Methods of Making Polypeptides

In some aspects, the polypeptides described herein (e.g., IL-2, such as extended-PK IL-2) are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

### Expression of Polypeptides

The nucleic acid molecules described above can be contained within a vector that is capable of directing their expression in, for example, a cell that has been transduced with the vector. Accordingly, in addition to polypeptide mutants, expression vectors containing a nucleic acid molecule encoding a mutant and cells transfected with these vectors are among the certain embodiments.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide mutant are also suitable for use. A cell is a transfected cell, i.e., a cell into which a nucleic acid molecule, for example a nucleic acid molecule encoding a mutant polypeptide, has been introduced by means of recombinant DNA techniques. The progeny of such a cell are also considered suitable for use in the methods disclosed herein.

The precise components of the expression system are not critical. For example, a polypeptide mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

### Pharmaceutical Compositions and Modes of Administration

In certain embodiments, IL-2 (e.g., extended-PK IL-2), a therapeutic antibody, a cancer vaccine, and optionally an immune checkpoint blocker, are administered together (simultaneously or sequentially). In certain embodiments, IL-2 (e.g., extended-PK IL-2) and a therapeutic antibody are administered together (simultaneously or sequentially). In certain embodiments, IL-2 (e.g., extended-PK IL-2) and a cancer vaccine are administered together (simultaneously or sequentially). In certain embodiments, IL-2 (e.g., extended-PK IL-2) and an immune checkpoint blocker are administered together (simultaneously or sequentially). In certain embodiments, IL-2 (e.g., extended-PK IL-2), a therapeutic antibody, a cancer vaccine, and optionally an immune checkpoint blocker, are administered separately. In certain embodiments, an antagonist of VEGF is used in place of an immune checkpoint blocker.

In certain embodiments, the invention provides for a pharmaceutical composition comprising IL-2 (e.g., extended-PK IL-2) with a pharmaceutically acceptable diluent, carrier, solubilizer, emulsifier, preservative and/or adjuvant, a pharmaceutical composition comprising a cancer vaccine with a pharmaceutically acceptable diluent, carrier, solubilizer, emulsifier, preservative and/or adjuvant, a pharmaceutical composition comprising a therapeutic antibody with a pharmaceutically acceptable diluent, carrier, solubilizer, emulsifier, preservative and/or adjuvant, or a pharmaceutical composition comprising an immune checkpoint blocker with a pharmaceutically acceptable diluent, carrier, solubilizer, emulsifier, preservative and/or adjuvant.

In certain embodiments, the invention provides for pharmaceutical compositions comprising IL-2 (e.g., extended-PK IL-2), a therapeutic antibody, a cancer vaccine, and optionally an immune checkpoint blocker, with a pharmaceutically acceptable diluent, carrier, solubilizer, emulsifier, preservative and/or adjuvant. In certain embodiments, each of the agents, e.g., IL-2 (e.g., extended-PK IL-2), therapeutic antibody, a cancer vaccine, and optionally an immune checkpoint blocker can be formulated as separate compositions. In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of IL-2 (e.g., extended-PK IL-2), the therapeutic antibody, the cancer vaccine, and the optional immune checkpoint blocker.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising IL-2 (e.g., extended-PK IL-2), a therapeutic antibody, a cancer vaccine, and optionally an immune checkpoint blocker (or an antagonist of VEGF), can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising IL-2 (e.g., extended-PK IL-2), a cancer vaccine, a therapeutic antibody, and optionally an immune checkpoint blocker (or an antagonist of VEGF), can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising IL-2 (e.g., extended-PK IL-2), a therapeutic antibody, a cancer vaccine, and optionally an immune checkpoint blocker (or an antagonist of VEGF), in a pharmaceutically acceptable vehicle. In certain embodiments, a vehicle for parenteral injection is sterile distilled water in which IL-2 (e.g., extended-PK IL-2), a therapeutic antibody, a cancer vaccine, and optionally an immune checkpoint blocker (or an antagonist of VEGF), are formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, IL-2 (e.g., extended-PK IL-2), a therapeutic antibody, a cancer vaccine, and optionally an immune checkpoint blocker (or an antagonist of VEGF) can be formulated as a dry powder for inhalation. In certain embodiments, an inhalation solution comprising IL-2 (e.g., extended-PK IL-2), a therapeutic antibody, a cancer vaccine, and optionally an immune checkpoint blocker (or an antagonist of VEGF) can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, IL-2 (e.g., extended-PK IL-2), a therapeutic antibody, a cancer vaccine, and optionally an immune checkpoint blocker (or an antagonist of VEGF) that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of IL-2 (e.g., extended-PK IL-2), the therapeutic antibody, the cancer vaccine, and the optional immune checkpoint blocker (or an antagonist of VEGF). In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of IL-2 (e.g., extended-PK IL-2), a therapeutic antibody, a cancer vaccine, and optionally an immune checkpoint blocker (or an antagonist of VEGF) in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving IL-2 (e.g., extended-PK IL-2), a therapeutic antibody, a cancer vaccine, and optionally an immune checkpoint blocker (or an antagonist of VEGF) in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising IL-2 (e.g., extended-PK IL-2) and/or one or more pharmaceutical compositions comprising a therapeutic antibody and/or a cancer vaccine and/or an immune checkpoint blocker (or an antagonist of VEGF) to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which IL-2 (e.g., extended-PK IL-2), the therapeutic antibody, the cancer vaccine, and optionally an immune checkpoint blocker (or an antagonist of VEGF), are being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage for IL-2 (e.g., extended-PK IL-2) can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg.

In certain embodiments, a typical dosage for a therapeutic antibody can range from about 1 mg/kg to up to about 1000 mg/kg or more, depending on the factors mentioned above. In certain embodiments, the dosage can range from 5 mg/kg up to about 1000 mg/kg; or 10 mg/kg up to about 1000 mg/kg; or 50 mg/kg up to about 1000 mg/kg.

In certain embodiments, a typical dosage for an immune checkpoint blocker can range from about 0.1 mg/kg to up to about 300 mg/kg or more, depending on the factors mentioned above. In certain embodiments, the dosage can range from 1 mg/kg up to about 300 mg/kg; or 5 mg/kg up to about 300 mg/kg; or 10 mg/kg up to about 300 mg/kg.

In certain embodiments, the frequency of dosing will take into account the pharmacokinetic parameters of IL-2 (e.g., extended-PK IL-2), the therapeutic antibody, the cancer vaccine, and optionally the immune checkpoint blocker in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising IL-2 (e.g., extended-PK IL-2), a therapeutic antibody, a cancer vaccine, and optionally an immune checkpoint blocker (or an antagonist of VEGF) in an ex vivo manner. In such instances, cells, tissues and/or organs that have been removed from the patient are exposed to a pharmaceutical composition comprising IL-2 (e.g., extended-PK IL-2), a therapeutic antibody, a cancer vaccine, and optionally an immune checkpoint blocker (or an antagonist of VEGF) after which the cells, tissues and/or organs are subsequently implanted back into the patient.

In certain embodiments, IL-2 (e.g., extended-PK IL-2), a therapeutic antibody, a cancer vaccine, and optionally an immune checkpoint blocker (or an antagonist of VEGF) can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Kits

A kit can include IL-2 (e.g., extended-PK IL-2), a cancer vaccine, a therapeutic antibody, and optionally an immune checkpoint blocker (or an antagonist of VEGF) as disclosed herein, and instructions for use. The kits may comprise, in a suitable container, IL-2 (e.g., extended-PK IL-2), a cancer vaccine, a therapeutic antibody, optionally an immune checkpoint blocker (or an antagonist of VEGF), one or more controls, and various buffers, reagents, enzymes and other standard ingredients well known in the art. Certain embodiments include a kit with IL-2 (e.g., extended-PK IL-2), a cancer vaccine, a therapeutic antibody, and optionally an immune checkpoint blocker (or an antagonist of VEGF) in the same vial. In certain embodiments, a kit includes IL-2 (e.g., extended-PK IL-2), a cancer vaccine, a therapeutic antibody, and optionally an immune checkpoint blocker (or an antagonist of VEGF) in separate vials.

The container can include at least one vial, well, test tube, flask, bottle, syringe, or other container means, into which IL-2 (e.g., extended-PK IL-2), a cancer vaccine, a therapeutic antibody, and optionally an immune checkpoint blocker (or an antagonist of VEGF) may be placed, and in some instances, suitably aliquoted. Where an additional component is provided, the kit can contain additional containers into which this component may be placed. The kits can also include a means for containing IL-2 (e.g., extended-PK IL-2), a cancer vaccine, a therapeutic antibody, and optionally an immune checkpoint blocker (or an antagonist of VEGF), and any other reagent containers in close confinement for commercial sale. Such containers may include injection or blow-molded plastic containers into which the desired vials are retained. Containers and/or kits can include labeling with instructions for use and/or warnings.

### Methods of Treatment

The IL-2 (e.g., extended-PK IL-2), cancer vaccine, therapeutic antibody, and optional immune checkpoint blocker (or an antagonist of VEGF), and/or nucleic acids expressing them, described herein, are useful for treating a disorder associated with abnormal apoptosis or a differentiative process (e.g., cellular proliferative disorders (e.g., hyperproliferaetive disorders) or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, e.g., IL-2 (e.g., extended-PK IL-2), a cancer vaccine, a therapeutic antibody, and optionally an immune checkpoint blocker (or an antagonist of VEGF), can be administered to a patient who has cancer.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The term “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The IL-2 (e.g., extended-PK IL-2), cancer vaccine, therapeutic antibody, and optional immune checkpoint blocker (or an antagonist of VEGF) can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

It will be appreciated by those skilled in the art that amounts for each of the IL-2 (e.g., extended-PK IL-2), cancer vaccine, therapeutic antibody, and optional immune checkpoint blocker (or an antagonist of VEGF) that are sufficient to reduce tumor growth and size, or a therapeutically effective amount, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

It will be appreciated by those skilled in the art that the B16 melanoma model used herein is a generalized model for solid tumors. That is, efficacy of treatments in this model is also predictive of efficacy of the treatments in other non-melanoma solid tumors. For example, as described in Baird et al. (J Immunology 2013; 190:469-78; Epub Dec. 7, 2012), efficacy of cps, a parasite strain that induces an adaptive immune response, in mediating anti-tumor immunity against B16F10 tumors was found to be generalizable to other solid tumors, including models of lung carcinoma and ovarian cancer. In another example, results from a line of research into VEGF targeting lymphocytes also shows that results in B16F10 tumors were generalizable to the other tumor types studied (Chinnasamy et al., JCI 2010;120:3953-68; Chinnasamy et al., Clin Cancer Res 2012; 18:1672-83). In yet another example, immunotherapy involving LAG-3 and PD-1 led to reduced tumor burden, with generalizable results in a fibrosarcoma and colon adenocarcinoma cell lines (Woo et al., Cancer Res 2012; 72:917-27).

In certain embodiments, the IL-2 (e.g., extended-PK IL-2), cancer vaccine, therapeutic antibody, and optional immune checkpoint blocker (or an antagonist of VEGF) disclosed herein are used to treat cancer.

In certain embodiments, the IL-2 (e.g., extended-PK IL-2), cancer vaccine, therapeutic antibody, and optional immune checkpoint blocker (or an antagonist of VEGF) disclosed herein are used to treat melanoma, leukemia, lung cancer, breast cancer, prostate cancer, ovarian cancer, colon cancer, and brain cancer.

In certain embodiments, the IL-2 (e.g., extended-PK IL-2), cancer vaccine, therapeutic antibody, and optional immune checkpoint blocker (or an antagonist of VEGF) disclosed herein inhibit the growth and/or proliferation of tumor cells.

In certain embodiments, the IL-2 (e.g., extended-PK IL-2), cancer vaccine, therapeutic antibody, and optional immune checkpoint blocker (or an antagonist of VEGF) disclosed herein reduce tumor size.

In certain embodiments, the IL-2 (e.g., extended-PK IL-2), cancer vaccine, therapeutic antibody, and optional immune checkpoint blocker (or an antagonist of VEGF) disclosed herein inhibit metastases of a primary tumor.

It will be appreciated by those skilled in the art that reference herein to treatment extends to prophylaxis as well as the treatment of the noted cancers and symptoms.

The present disclosure is further illustrated by the following examples, which should not be construed as further limiting. The contents of all figures and all references, Genbank sequences, patents and published patent applications cited throughout this application are expressly incorporated herein by reference. In particular, the disclosures of PCT publication WO 13/177187, U.S. Patent No. 8,536,301, and U.S. Patent Publication No. 2014/0073518 are expressly incorporated herein by reference.

### EXAMPLES

Below are examples of specific embodiments for carrying out the methods described herein. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992). Moreover, while the examples below employ extended-PK IL-2 of mouse origin (i.e., both the extended-PK group (mouse serum albumin) and IL-2 are of mouse origin), it should be understood that corresponding human extended-PK IL-2 (i.e., human serum albumin (HSA) and human IL-2, and variants thereof) can be readily generated by those of ordinary skill in the art using methods described supra, and used in the methods disclosed herein.

### Example 1

### Synergistic Tumor Control and Survival With Triple Combination Therapy

To assess the effectiveness of combination treatment in cancer, the B16F10 melanoma mouse model was utilized. 1×10⁶ B16F10 melanoma cells (ATCC), which are poorly immunogenic and aggressively form tumors, were subcutaneously injected into C57BL/6 mice. Immunotherapy was administered 8, 15, 22, 29, and 36 days after tumor inoculation. This consisted of 100 μg TA99 (an anti-Trp-1 antibody, produced by researcher) and 30 μg mouse serum albumin (MSA)-IL-2 (produced by researcher).

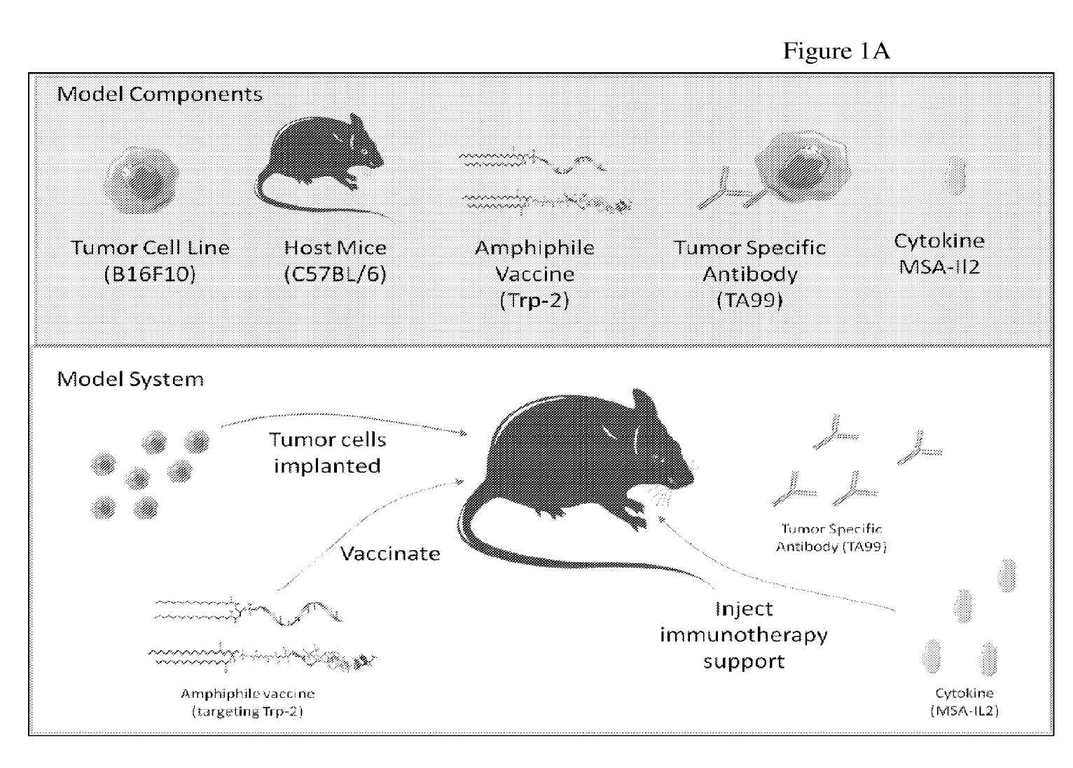

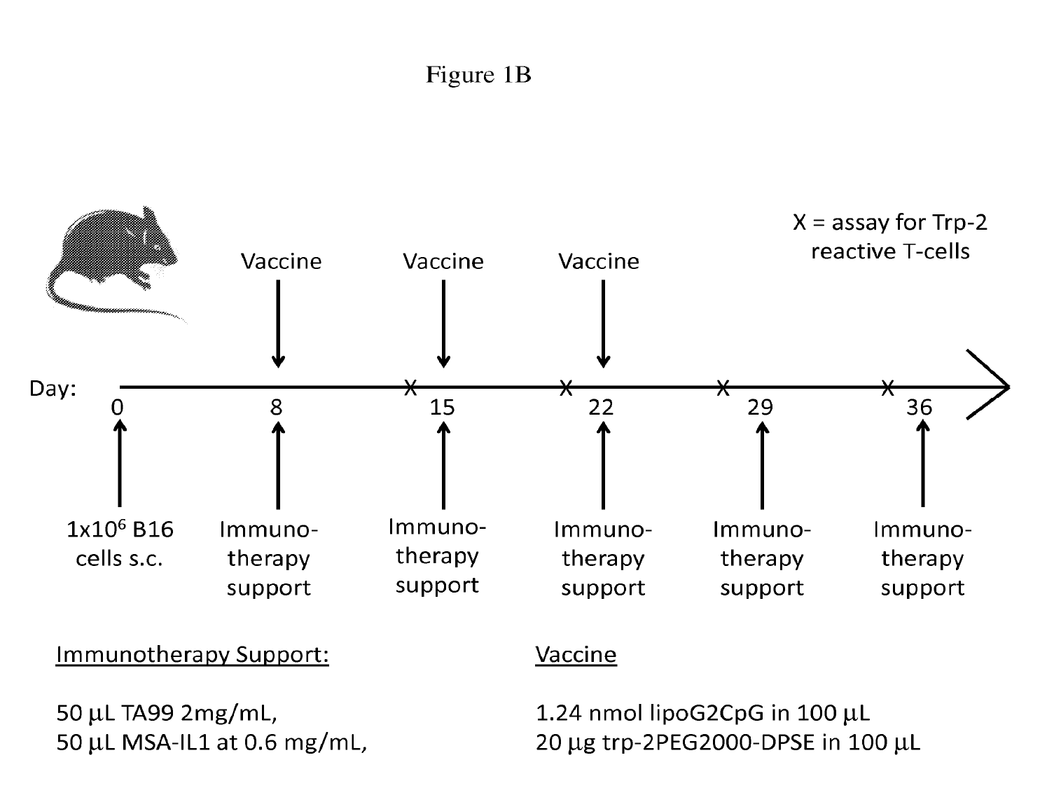

The amphiphile cancer vaccine targeting Trp-2 was administered on days 8, 15, and 22 after inoculation of B16F10 cells. Oligonucleotide amphiphiles were synthesized using an ABI 394 synthesizer on a 1.0 μmol scale. All lipophilic phosphoramidites were conjugated as a final ‘base’ on the 5′end of oligonucleotides. Lui, H. et al., Angew. Chem. Int. Ed. Engl. 50, 7052-7055 (2011). A lymph-node targeted molecular adjuvant was made in which a 20 base phosphorothioate (PS)-stabilized CpG oligonucleotide was linked at the 5′ to diacyl lipid via a guanine linker (lipo-G₂-CpG) as described in Liu, H. et al., Nature 507: 519-522 (Mar. 27, 2014). The tumor-associated self-antigen Trp2 from melanoma was conjugated to 1,2-distearoyl-sn-glycero-3-phophoethanolamine-N-PEG (DSPE-PEG 2kDa) to generate amph-peptides for vaccination studies. Antigen amphiphiles were synthesized by reacting N-terminal cysteine-modified peptides with maleimide-PEG₂₀₀₀-DSPE in dimethly formamide. A schematic of the treatment regimen is shown in FIGS. 1A and 1B.

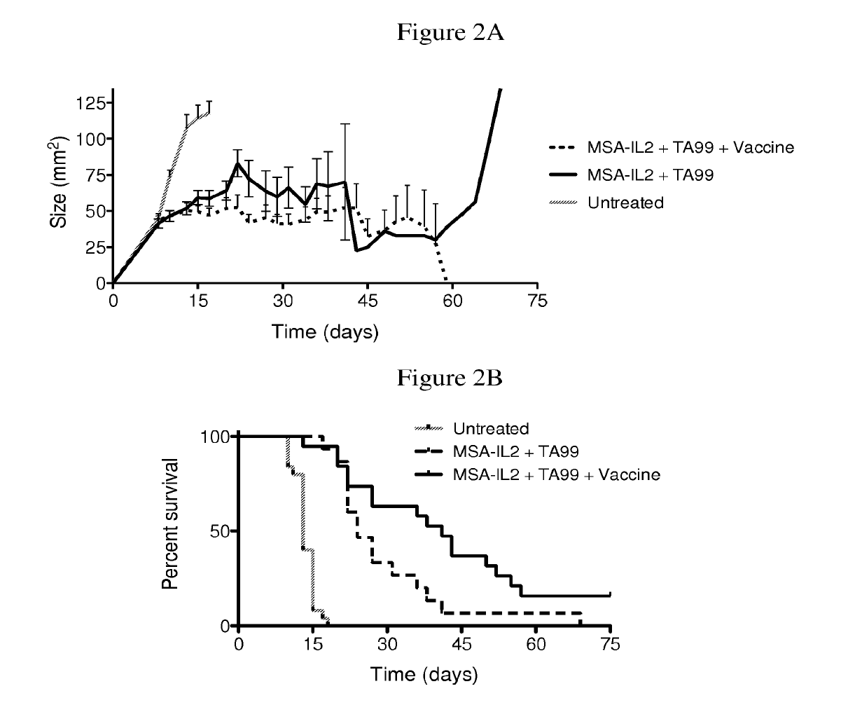

Tumor area was measured throughout the course of the experiment and is summarized in FIG. 2A. Synergistic reduction of tumor growth was observed when all 3 components (i.e., cancer vaccine, TA99, and MSA-IL-2) were administered, relative to the double combination (MSA-IL-2 + TA99) and vehicle (PBS).

Survival was examined throughout the course of the experiment and plotted in FIG. 2B. Survival was substantially improved with the triple combination (i.e., MSA-IL-2 + TA99+ vaccine) relative to the double combination.

### Example 2

### Vitiligo With Triple Combination Therapy

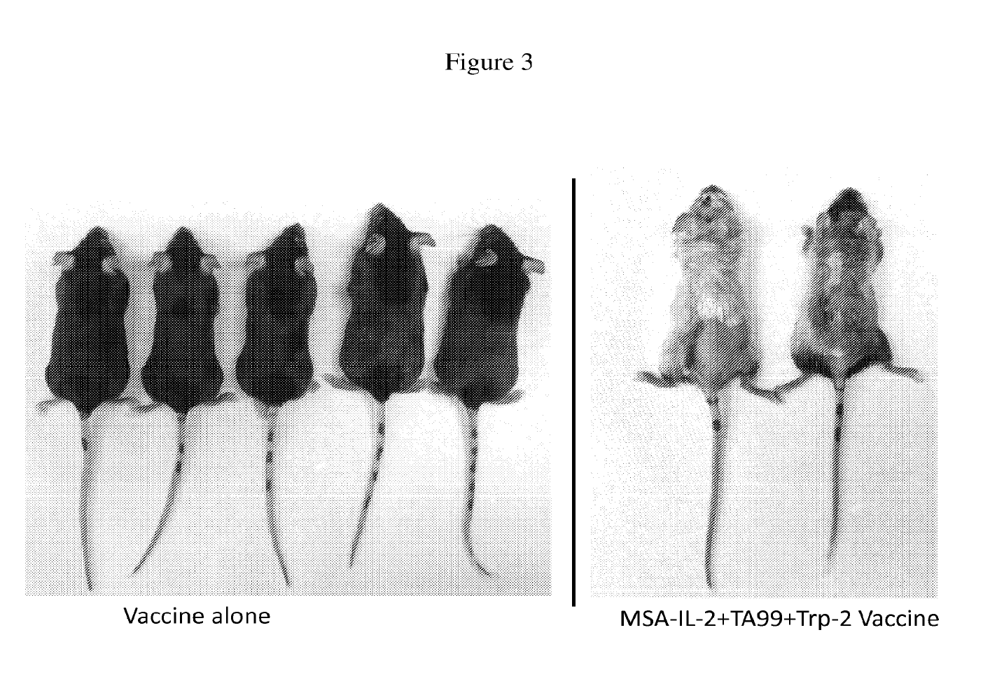

To assess the immune response to the various combination therapies, mice inoculated with B16F10 cells and subsequently treated as described in Example 1, were observed for vitiligo, a depigmentation of the skin, 55 days after tumor inoculation. Control mice were age matched and treated with vaccine alone with no inoculation of tumor cells. FIG. 3 shows that surviving mice treated with the triple combination (i.e., MSA-IL-2 + TA99 + Trp-2 vaccine) displayed vitiligo, whereas control mice did not. This indicates a potent and sustained immune response against the melanoma tumors. Vitiligo has long been an established positive prognostic factor in clinical outcomes of melanoma patients (Quaglino, 2010).

### Example 3

### Antigen-Reactive CD8+ T Cells in Mice Treated With Combination Therapy

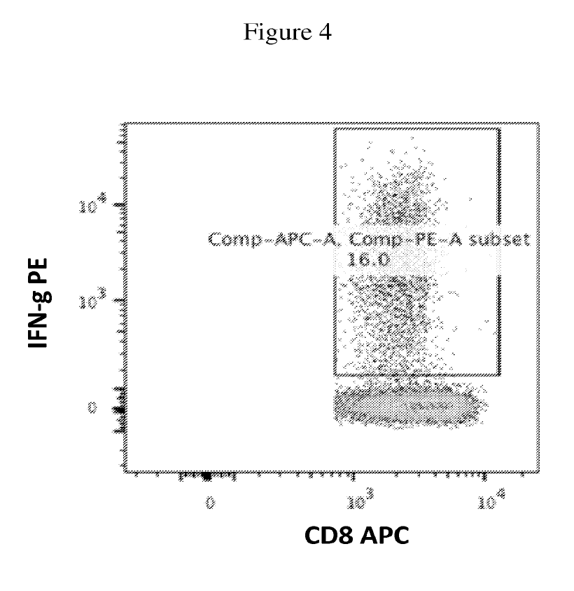

In this experiment, the reactivity of CD8+ T cells to the Trp-2 antigen administered by way of vaccine was assessed. To measure antigen-reactive T cells, peripheral blood mononuclear cells (PBMCs) were isolated from mice inoculated with B16F10 cells the day before each treatment and then once a week for the duration of the study. PBMCs were incubated in media containing 0.1 mg/mL Trp-2 peptide for 2 hours at 37° C. Brefeldin A was then added to the cells and incubated at 37° C. for another 4 hours. After peptide incubation, cells were washed and stained for CD8 for 30 minutes at 4° C. The cells were then washed, fixed, and permeabilized before being stained for IFNγ for 30 minutes at 4° C. Cells were then washed and analyzed on a flow cytometer. FIG. 4 depicts a representative readout for the Trp-2 assay and how the positive cells were determined.

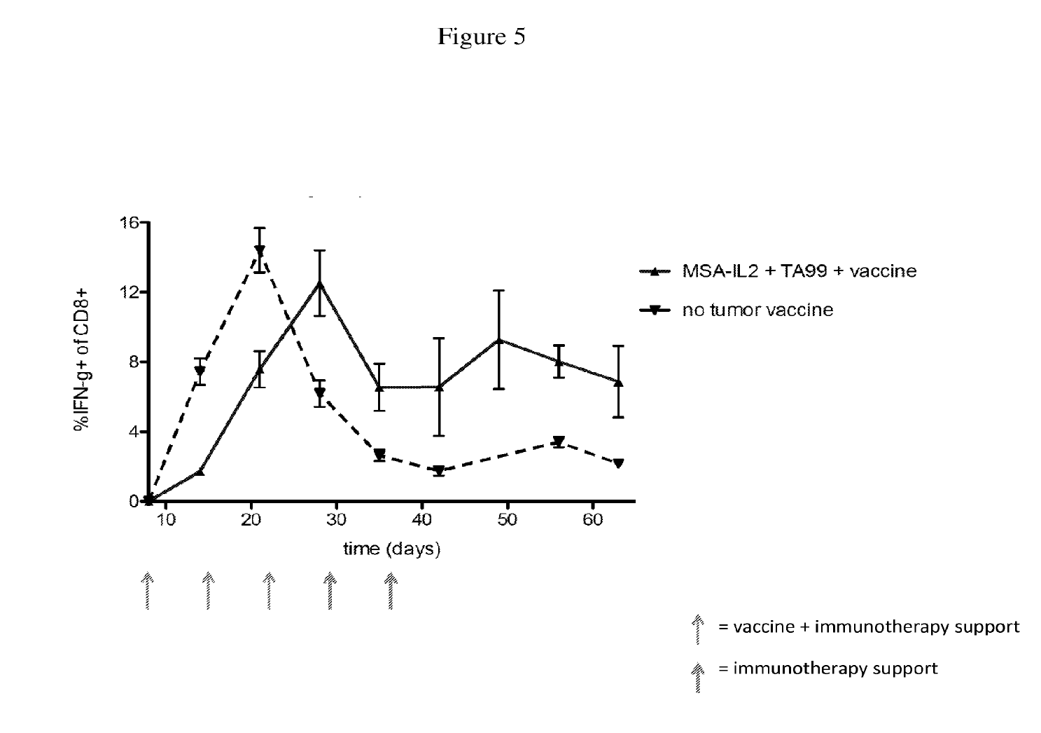

The percentage of IFNγ producing CD8+ T cells was compared between the control (no tumor vaccine) and combination treatment groups. As shown in FIG. 5, the percentage of IFNγ producing CD8+ T cells reactive to Trp-2 was maintained over time, where MSA-IL-2, TA99, and cancer vaccine treatment resulted in increased reactive T cells compared to cells not treated with the cancer vaccine (for up to 70 days after inoculation of B16F10 cells).

### Example 4

### Enhanced Tumor Control and Survival by Addition of Immune Checkpoint Blocker (Quadruple Combination)

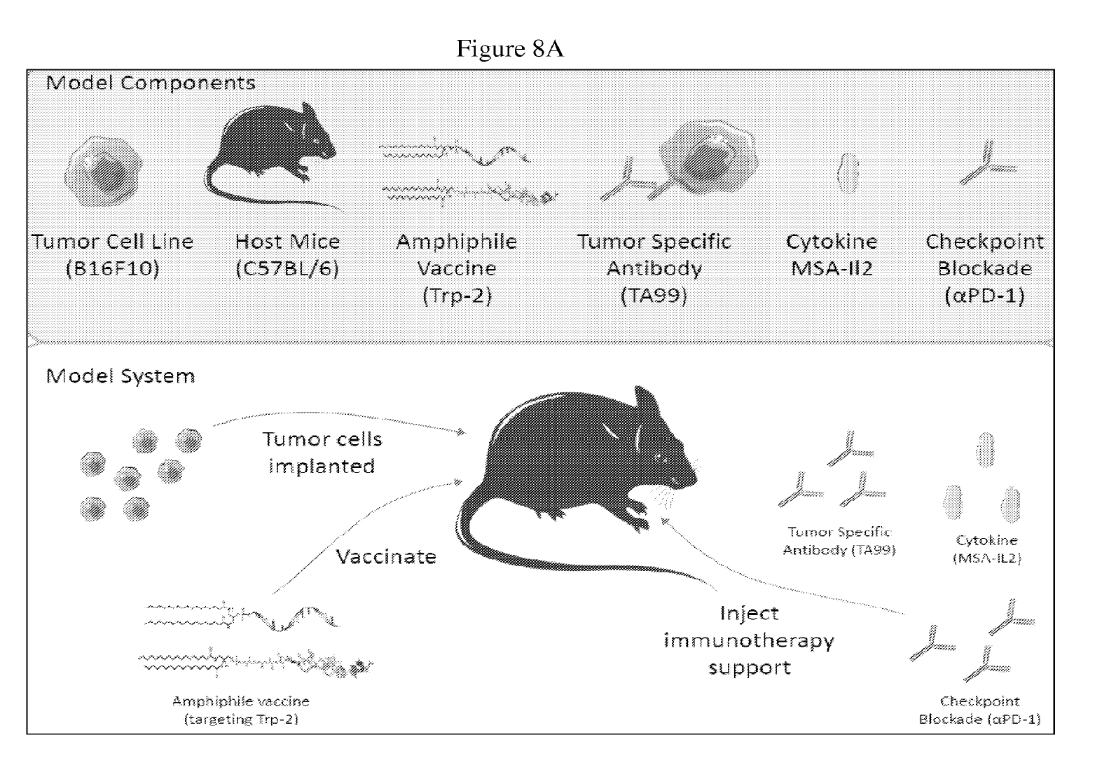

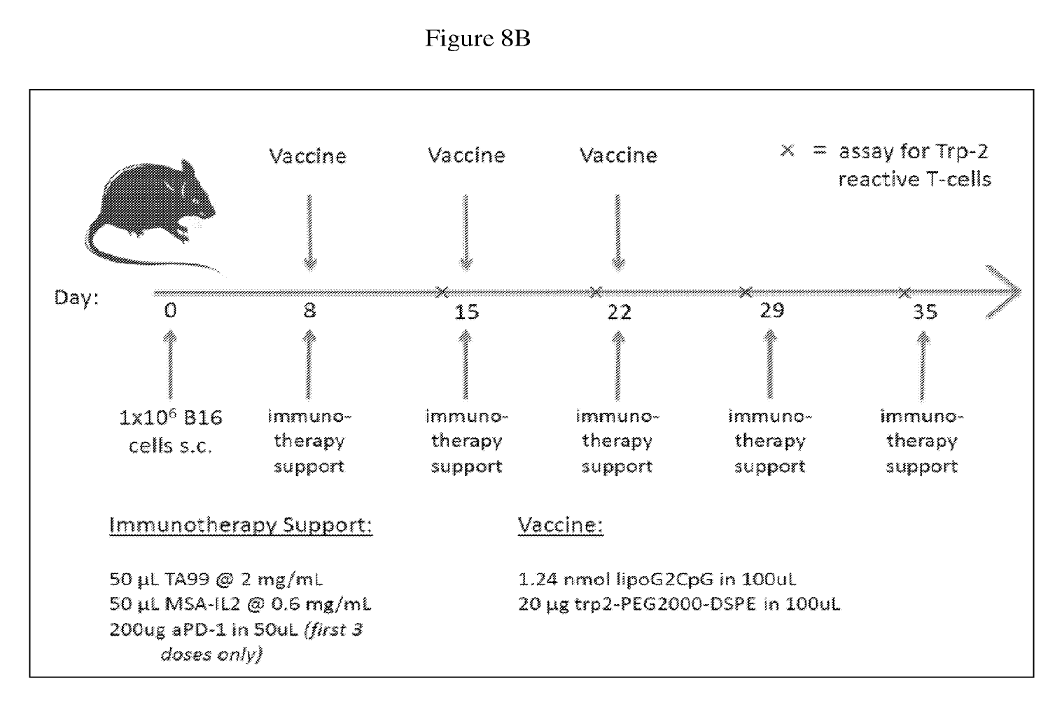

To assess the effectiveness of combination treatment which additionally includes an immune checkpoint blocker, the B16F10 melanoma was utilized as described in Example 1. In addition to the administration of MSA-IL-2, TA99 and cancer vaccine, 200 μg anti-PD-1 antibody (clone RMP1-14 from BioXcell) was administered on days 8, 15, 22, 29 and 35 after inoculation of B16F10 cells. A schematic of the treatment regimen is found in FIG. 8B.

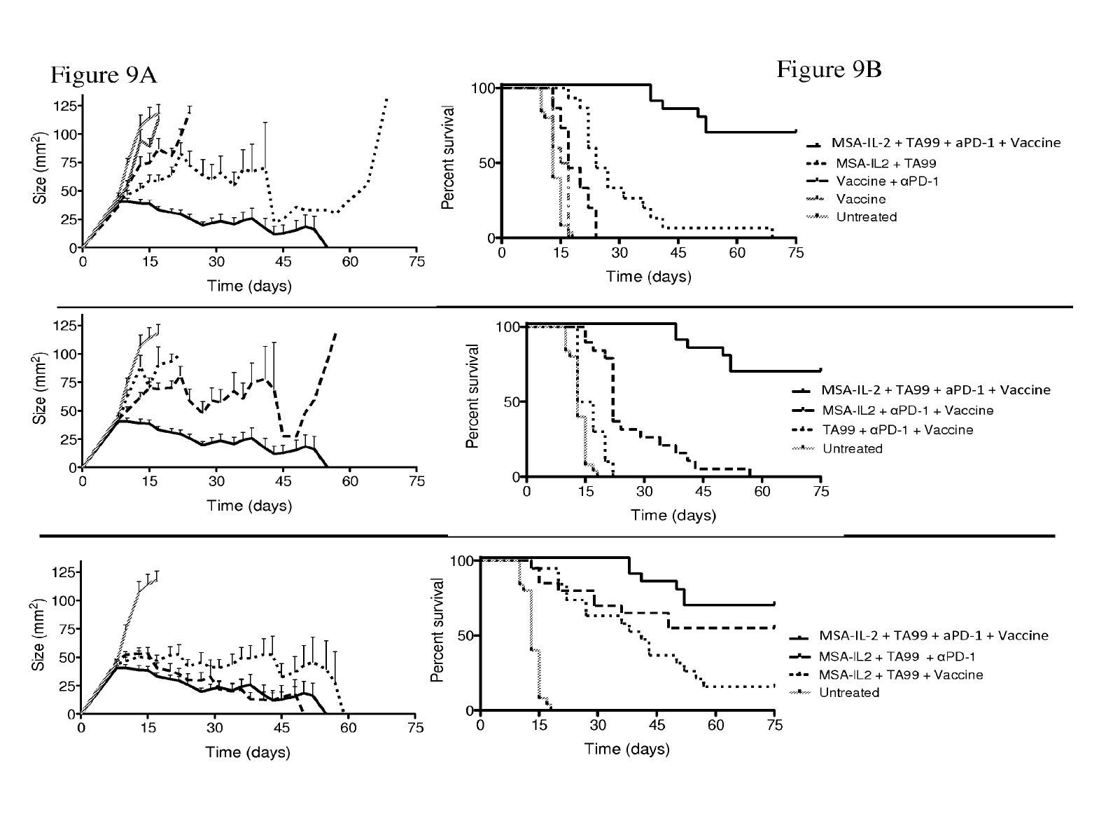

Tumor area was measured throughout the course of the experiment and is summarized in FIG. 9A. Synergistic reduction of tumor growth was observed when all 4 components (i.e., cancer vaccine, anti-PD1 antibody, TA99, and MSA-IL-2) were administered. Tumor growth was also controlled with the triple combinations (i.e., MSA-IL-2 + TA99 + vaccine and MSA-IL-2 + TA99 + anti-PD-1 antibody), relative to the double combinations (anti-PD-1 antibody+TA99, anti-PD-1 antibody+vaccine, anti-PD-1 antibody+MSA-IL-2, and MSA-IL-2 + TA99) and vehicle (PBS).

Survival was also examined and plotted in FIG. 9B. Survival was substantially improved with the quadruple combination (i.e., MSA-IL-2 + anti-PD1 antibody+TA99+vaccine). The triple combinations (MSA-IL-2 + TA99 + vaccine and MSA-IL-2 + TA99 + anti-PD-1 antibody) also substantially improved survival relative to the double and single combinations. The quadruple combinationwas not overtly toxic as animals were otherwise healthy and steadily gained weight comparable to control animals (data not shown).

### Example 5

### Quadruple Combination Treated Mice Survive Secondary Challenge

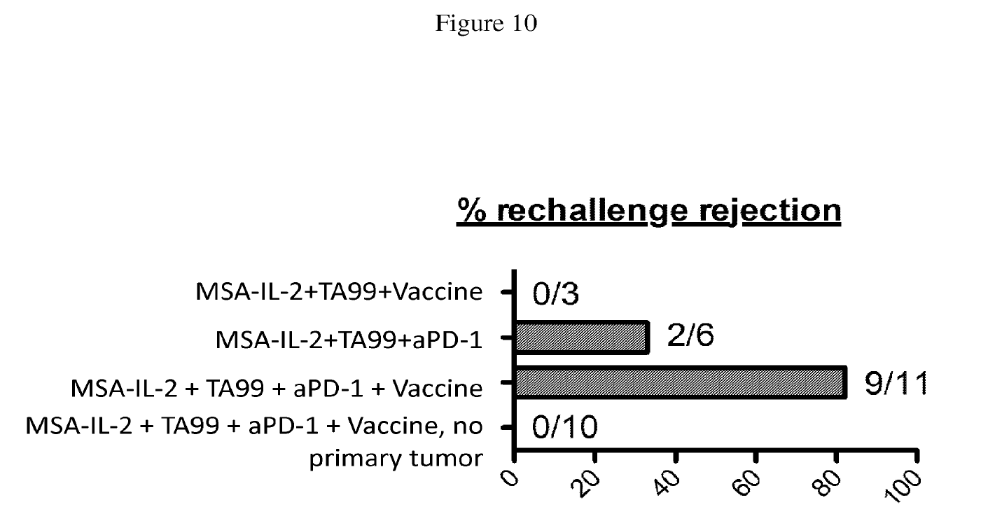

To determine the effect of adding an immune checkpoint blocker to the triple combination (i.e., MSA-IL-2, TA99, and cancer vaccine) on the memory of immune cells against tumor cells, surviving mice that underwent the regimen described in Example 4 were re-challenged 75 days after initial tumor inoculation. These mice were injected with 100,000 B16F10 cells and survival was monitored. Mice originally treated with MSA-IL-2 + anti-PD1 antibody+TA99 + vaccine survived the re-challenge (FIG. 10). At 35 days post-secondary challenge, none of the remaining mice had visible tumors and were deemed to have rejected the secondary tumors.

### Example 6

### Vitiligo With Quadruple Combination Therapy

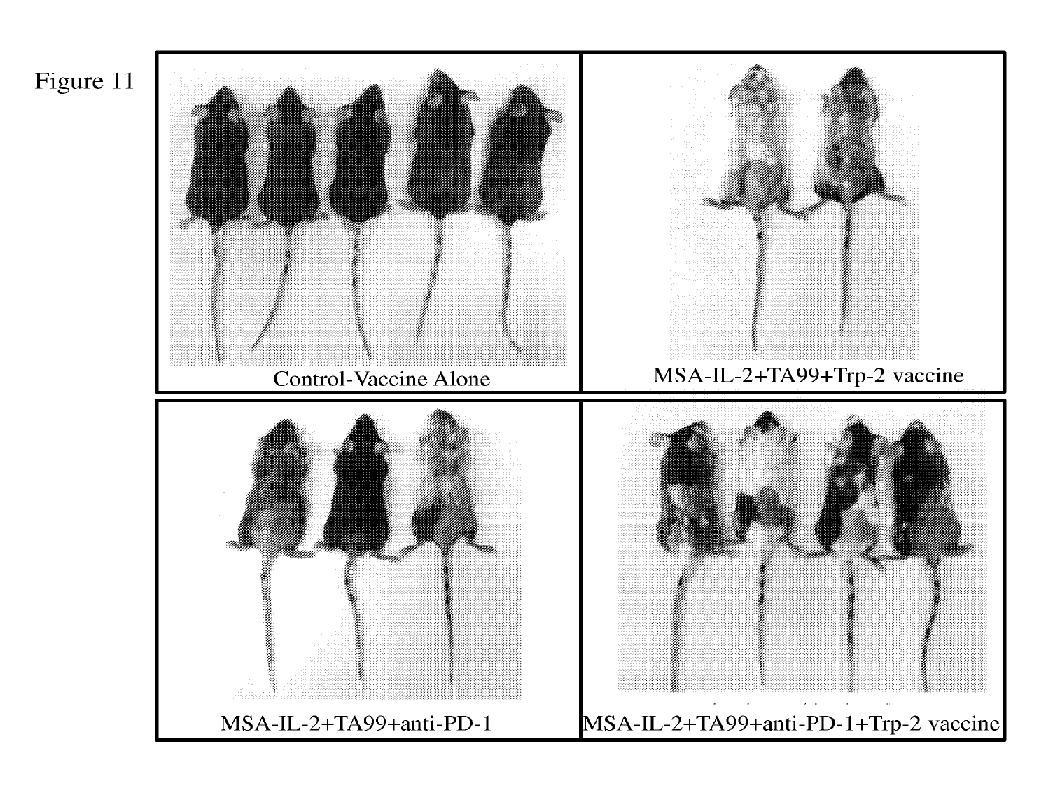

To assess the immune response when tumor-bearing mice are treated with the quadruple combination therapy, mice inoculated with B16F10 cells and subsequently treated as described in Example 4 were observed for vitiligo, 55 days after tumor inoculation. Control mice were age matched and treated with vaccine alone with no inoculation of tumor cells. FIG. 11 shows that surviving mice treated with the quadruple combination (i.e., MSA-IL-2+ TA99+anti-PD-1 antibody+Trp-2 vaccine) and triple combinations (i.e., MSA-IL-2+TA99+Trp-2 vaccine and MSA-IL-2+TA99 +anti-PD1 antibody) all displayed vitiligo, whereas control mice did not. This indicates a potent and sustained immune response against Trp-2, a melanocyte antigen, wherein targeting this antigen results in depigmentation.

### Example 7

### Antigen-Reactive CD8+ T Cells in Mice Treated With Quadruple Combination Therapy

To assess the role of an immune checkpoint blocker on the reactivity of CD8+ T cells to the Trp-2 antigen administered by way of vaccine, CD8+ T cells were measured as described in Example 3.

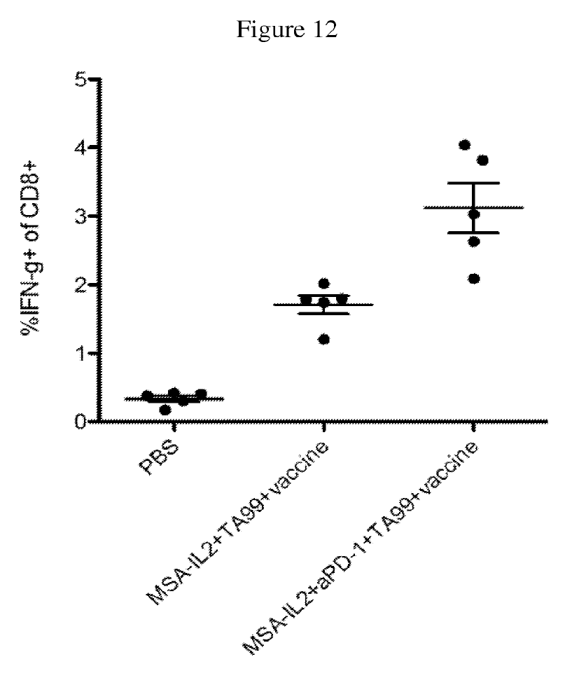

The percentage of IFNγ producing CD8+ T cells was compared between treatment combinations. As shown in FIG. 12, the triple combination of MSA-IL-2 + TA99 + vaccine resulted in about 2% reactive T cells, whereas the quadruple combination of MSA-IL-2 + anti-PD-1 antibody+TA99 + vaccine resulted in about 3% reactive T cells after one treatment. This result indicates that inclusion of the anti-PD-1 antibody increased the number of reactive T cells 14 days after tumor inoculation (i.e., 6 days after the first treatment). This was also observed over the course of 70 days after inoculation of B16F10 cells. FIG. 13 shows that the percentage of IFNγ producing CD8+ T cells reactive to Trp-2 is maintained over time, where MSA-IL-2+anti-PD-1 antibody+TA99+cancer vaccine treatment resulted in increased reactive T cells compared to vaccine alone or MSA-IL-2+TA99+cancer vaccine treatment.

### Example 8

### Response to Rechallenge in Mice Without Primary Tumors

To further assess the immune response with the quadruple combination therapy (i.e., MSA-IL-2+TA99+ anti-PD-1 antibody+vaccine) in mice after tumor challenge, mice inoculated with or without B16F10 cells, subsequently treated as described in Example 4, and then rechalleneged as described in Example 5, were observed for Trp-2 reactive T cells and vitiligo.

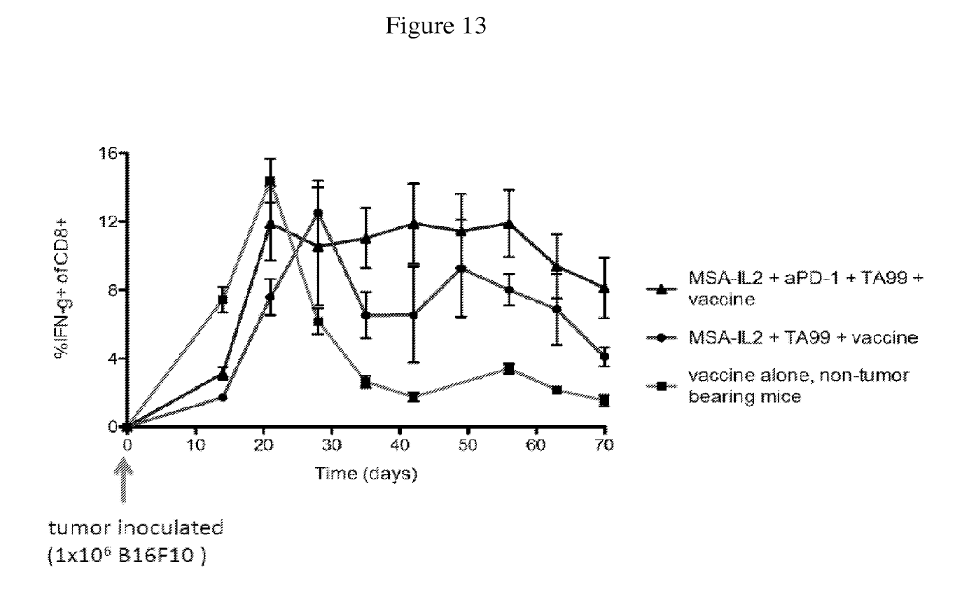

Unlike equivalently treated mice that were inoculated with primary tumors, mice treated with the quadruple combination, but with no primary tumors, were unable to reject subsequent tumor challenge. 9 out of 11 mice rejected subsequent tumor challenges if they had a primary tumor, whereas 0 out of 5 mice rejected subsequent tumor challenges if they did not have a primary tumor. FIG. 14 shows that the T cell response was tumor antigen dependent and boosted after the subsequent rechallenge. Mice without primary tumors and treated with the quadruple combination did not sustain high levels of Trp2 reactive T cells after rechallenge. Mice treated with the quadruple combination showed a stronger rechallenge response than equivalently treated mice without primary tumors. This may explain why mice without primary tumors were unable to reject subsequent tumor challenges.

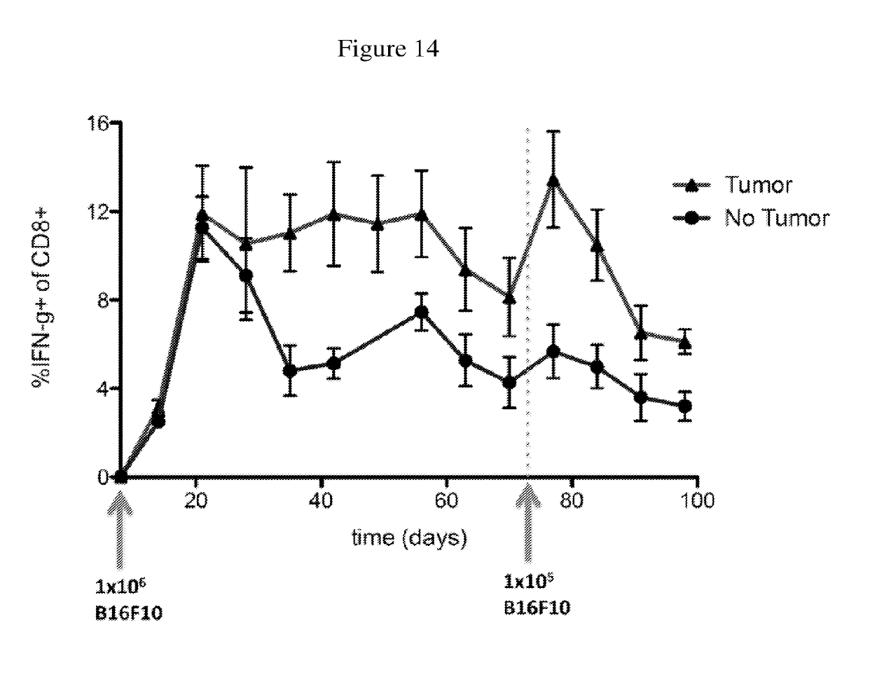

Despite this lack of protection from rechallenge, mice without primary tumors underwent strong vitiligo responses, normally associated with successful immunotherapies (FIG. 15). The dissociation between vitiligo and successful immunotherapties has been observed previously (Byrne et al., J. Immunol. (2014), Vol 192: 1433-1439). These results highlight the importance of the tumor-derived antigen and suggest that cross-presentation of that antigen may have led to antigen spreading. It also suggests that vitiligo may indicate a strong immunological response to targeted melanoma differentiation markers, but a complex, suppressive tumor microenvironment may overcome this response.

### Example 9

### Immune Cell Populations Important for Survival in Mice Treated With Quadruple Combination

To assess the role of various immune cell populations in the observed improved survival of mice with B16F10 tumors treated with the quadruple combination (i.e., MSA-IL-2+TA99+anti-PD-1 antibody+vaccine), depletion antibodies were administered twice a week starting one day prior to the first treatment. Cytotoxic lymphocytes were depleted with an anti-CD8α antibody (clone 2.43). Natural killer cells were depleted with an anti-NK1.1 antibody (clone PK136), and neutrophils were depleted with an anti-Ly-6G antibody (clone 1A8). These antibodies were administered at 400 μg per dose. All antibodies were purchased from BioXcell. Cross-presenting dendritic cells were depleted by using Batf3 −/− mice. Batf3 −/− mice lack the function of the basic leucine zipper transcription factor, ATF-like 3. Deletion of Batf3 has been shown to prevent the development of CD8+ dendritic cells, which are important for the cross-presentation of exogenous antigen on MHC Class I.

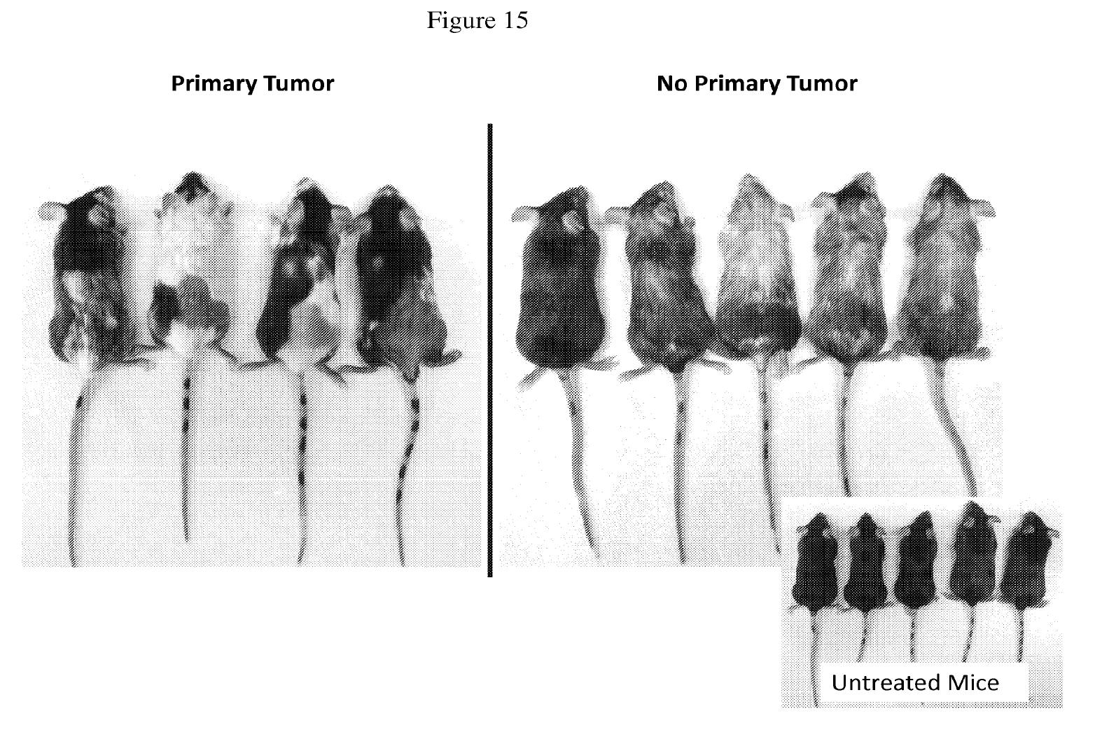

FIG. 16 shows the survival of mice treated with the quadruple combination without the various immune cells. CD8+ T cells and cross-presenting dendritic cells were identified as two critical cell types contributing to the potentcy of the quadruple combination therapy. Natural killer cells and neutrophils were not essential, but their depletion led to significant reductions in overall survival rates. A notable result of these depletion experiments is that the vaccine and checkpoint blockade therapies are marginalized since they primarily act through T cell mediated pathways. More generally, these results suggest that the adaptive immune system is a critical part of the quadruple combination immunotherapy.

### Example 10

### Immune Cell Infiltration in Tumors

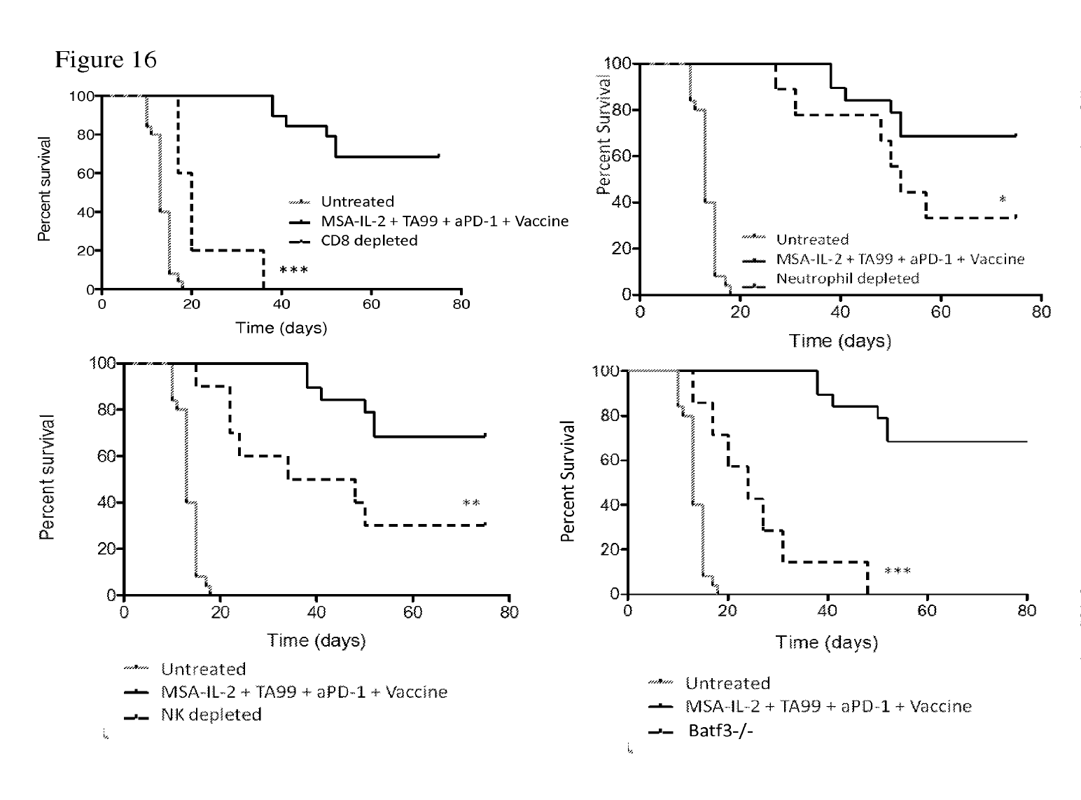

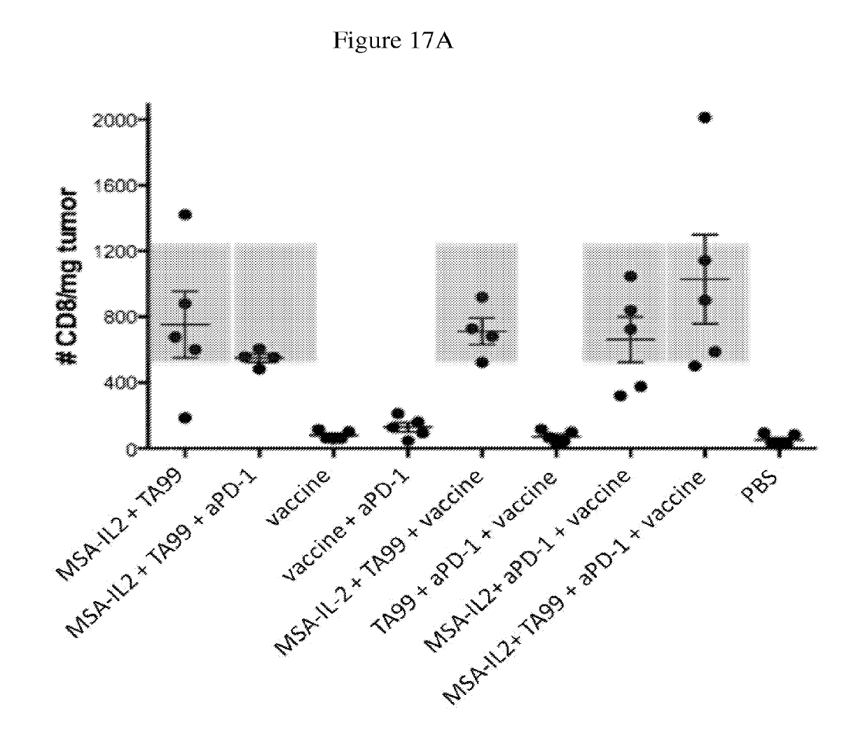

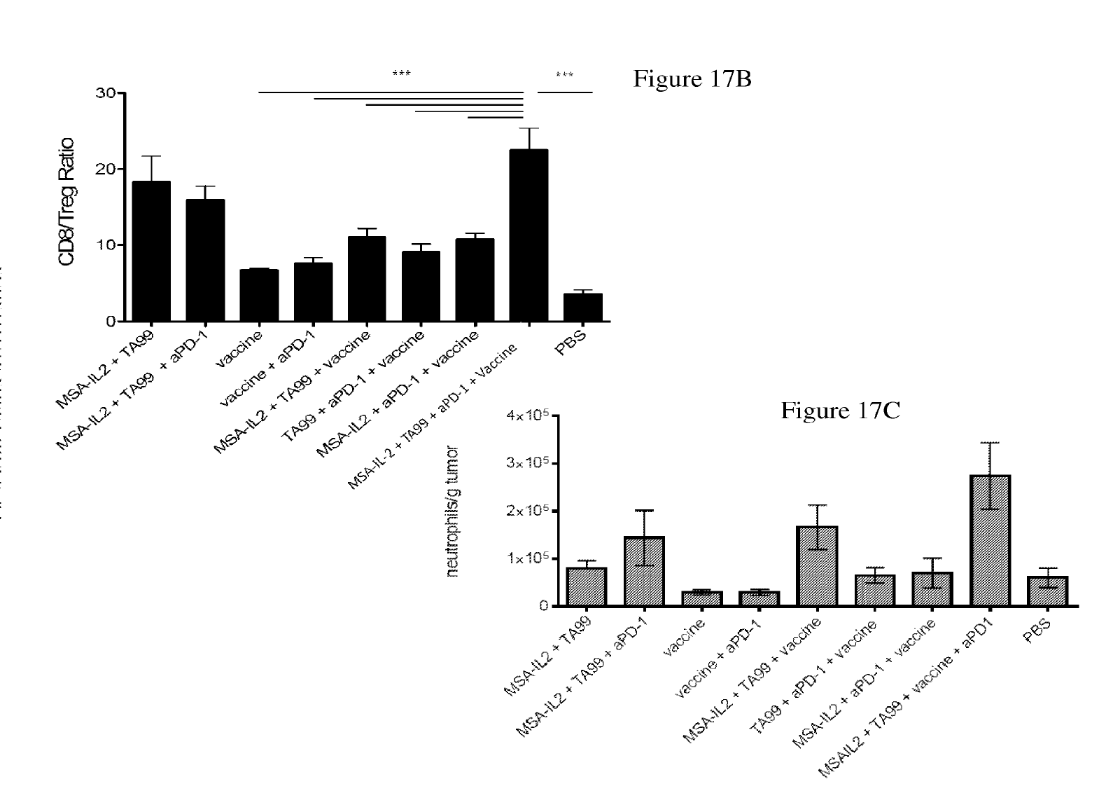

To assess the role of immune cell populations in the control of tumor growth in mice treated with the various combination therapies, mice inoculated with B16F10 cells were sacrified and tumors harvested 1-3 days after a single dose of the different combination therapies. The number of CD8+ T cells, CD4+ T cells (regulatory or non-regulatory), neutrophils, natural killer cells and dendritic cells in the tumors were measured via flow cytometry as previously described (Zhu et al., Cancer Cell (2015) Vol 27: 489-501). CD8+ T cells were critical to the efficacy of the treatments as shown in Example 9. Their role in tumor control was confirmed by the observation of high levels of infiltrates in effective combinations (FIG. 17A). FIG. 17B shows the ratio of CD8 to Treg cells, which is considered an accurate indicator of an effective immune response. An increased ratio correlated with successful therapies. In addition, the total number of dendritic cells was enhanced by administration of MSA-IL-2 (data not shown). Infiltrating neutrophils were signficiantly increased in the most effective treatments (i.e., MSA-IL-2+TA99+anti-PD-1 antibody+vaccine, MSA-IL-2+TA99+vaccine, and MSA-IL-1+TA99+anti-PD-1 antibody) (FIG. 17C). This further demonstrated the need to engage both the adaptive and innate immune systems in effective immunotherapy combinations.

### Example 11

### Cross-Presentation of Antigens and Antigen Spreading

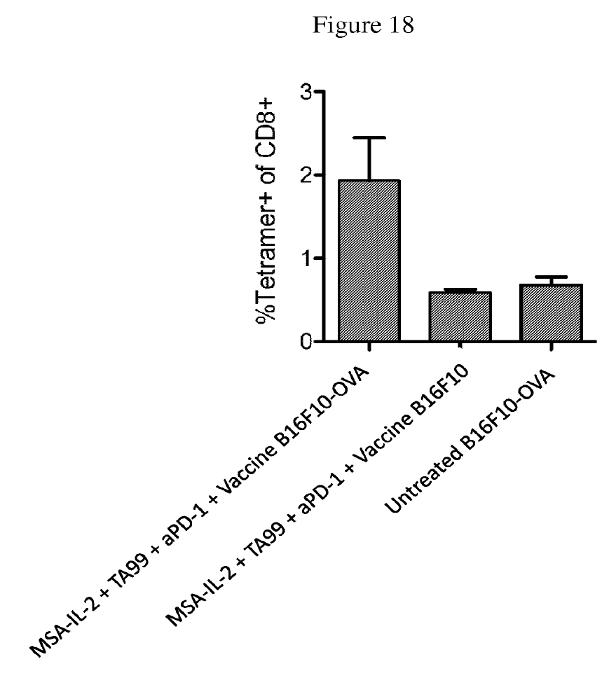

To assess the ability of the quadruple combination to induce antigen spreading facilitated by cross-presentation of tumor-derived antigen, OVA was used as a surrogate. B16F10-OVA cells were used to inoculate tumors in B6 mice, which were then treated with the quadruple combination (i.e., MSA-IL-2+TA99+anti-PD-1 antibody+vaccine) as described in Example 4. Tetramers complexed with OVA peptides were purchased from MBL. Tetramer staining was performed in buffer containing 50 nM dasatinib. 21 days after tumor inoculation, T cells were analyzed for OVA-specific T cell receptor expression by tetramer staining and flow cytometry. A significant response to OVA peptide was observed by flow cytometry, despite specifically not targeting the OVA antigen (FIG. 18). Because none of the therapies targeted OVA or were specifically engineered to elicit an OVA-specific response, the immune system itself developed this response. This indicated that cross-presentation of tumor derived antigens was occurring as a consequence of the combination immunotherapy.

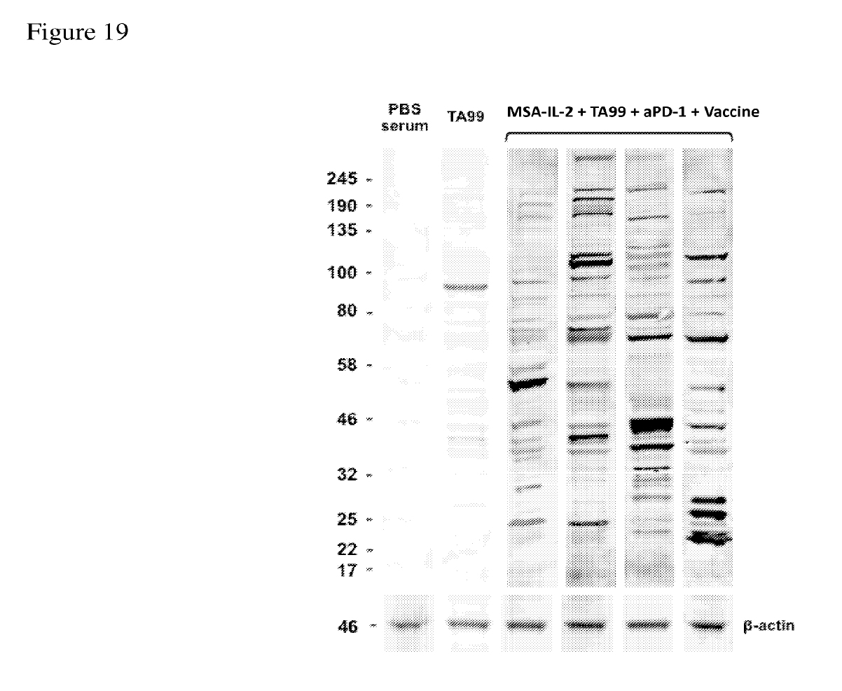

Antigen spreading occurs when the immune system identifies novel epitopes against the targeted tumor and raises an adaptive response against them. It is highly effective in curing established tumors and preventing recurrence (Corbiere et al., Cancer Res. (2011) Vol 71: 1253-1262). To test for antigen spreading following the quadruple treatment, B16F10 cell lysate was run on an SDS-PAGE gel (FIG. 19). Serum from quadruple combination-treated mice was used to probe the cell lysate for binding. Untreated serum from naïve mice was used as a control. After secondary binding and imaging, a robust humoral response was observed in treated mice compared to untreated mice. Serum was also collected post-secondary challenge (i.e., 100 days after tumor inoculation). A clear increase in response was observed after rechallenge, consistent with the CD8+ T cell response boost. These findings demonstrate the engagement of the humoral immune system against tumor antigens via antigen spreading.

### Example 12

### Inducible Tumor Model for Quadruple Combination Therapy

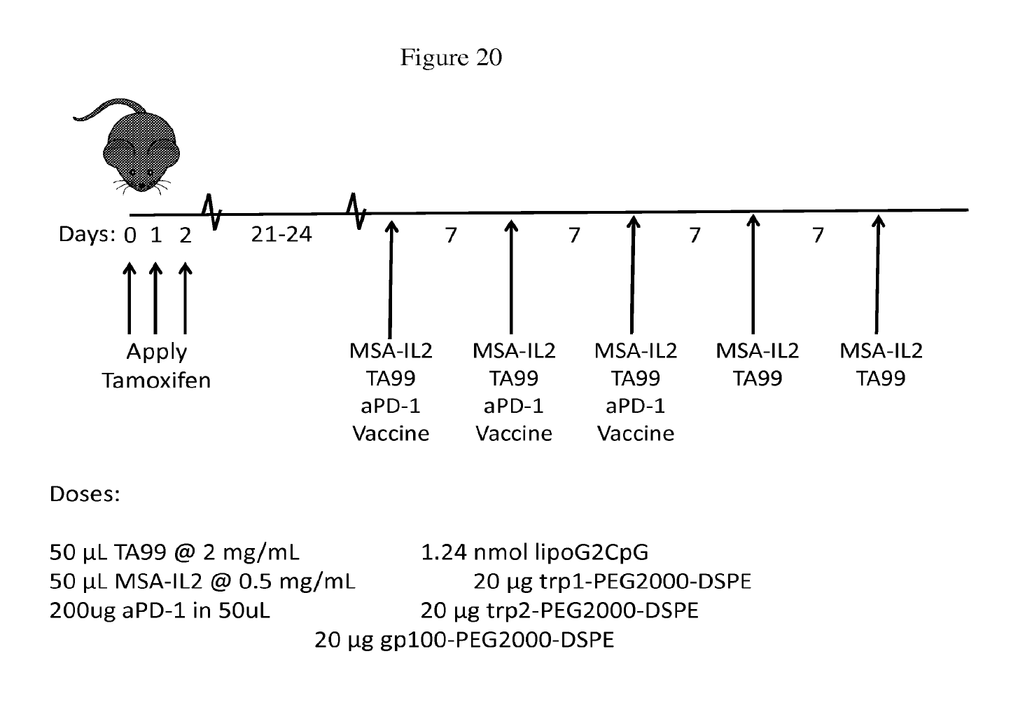

To further assess the efficacy of the quadruple combination treatment, the BRaf/Pten inducible tumor system was used. BRaf/Pten mice (Dankort et al., Nat. Genet. (2009) Vol 41: 544-552) were crossed with mT/mG (Muzumdar et al., Genes (2007) Vol 45: 593-605) to generate BRaf/Pten-TG mice. To induce tumors, 2 μL of 5 mg/mL tamoxifen was administered to the left ear on three consecutive days. 24-26 days later, when visible tumor lesions were present, treatment was begun and executed as described for the subcutaneous tumor model (FIG. 20). The vaccine used was a combination of three amph-peptides (15 μg amph-gp100, 15 μg amph-Trp-1, and 15 μg amph-Trp2) and 1.24 nmol amph-CpG, administered as a single dose. Mice were euthanized when pigmented lesions covered the induced ear (about 90% coverage) or when apigmented tumors reached 10 mm in diameter.

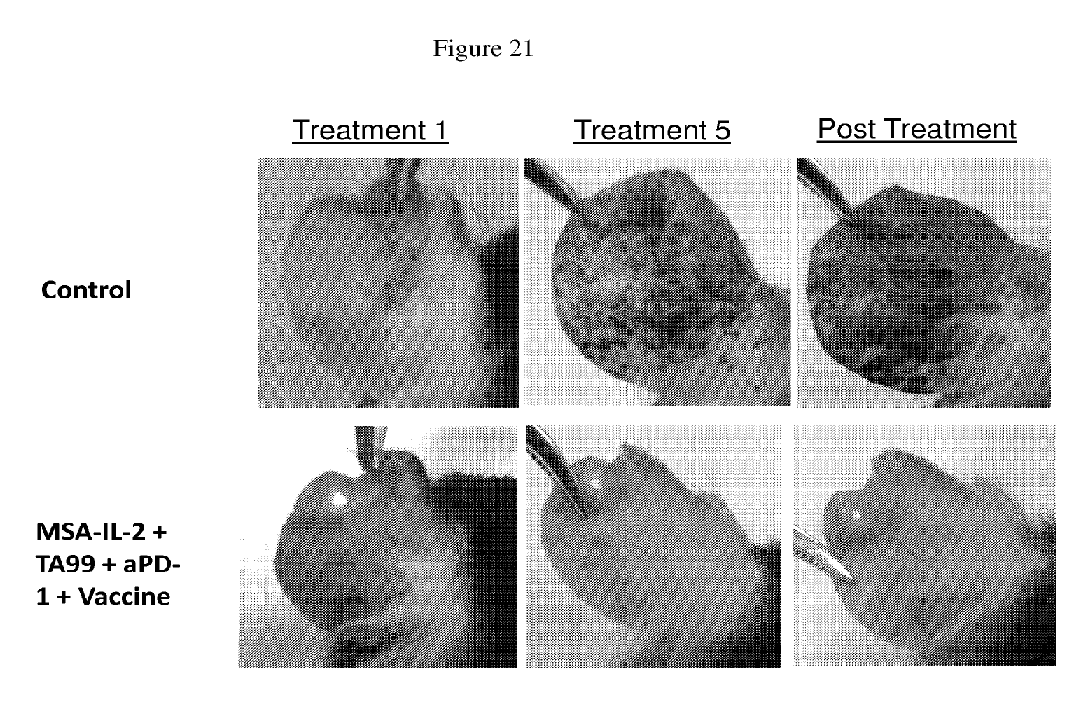

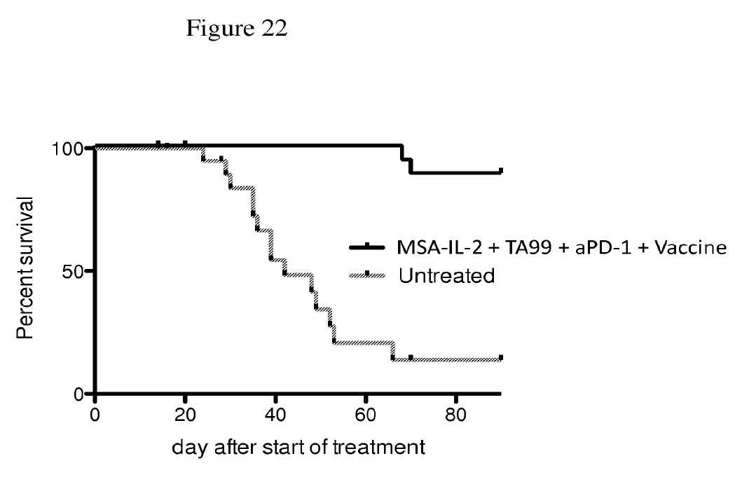

The quadruple combination therapy was effective at controlling the initial pigmented lesions, as shown in FIG. 21. These images are representative of the level of response achieved during the first 60 days of tumor establishment and treatment. By day 60, almost all of the untreated mice had complete coverage of their ears by the pigmented tumor cells. In contrast, lesions in quadruple combination-treated mice became smaller and in many cases disappeared. Overall survival of BRaf/Pten-TG mice also significantly improved with the quadruple combination treatment (FIG. 22). Eventually, however, apigmented tumors appeared in approximately half of the treated mice and grew progressively until euthanasia criteria were met. This may indicate that in more complex tumor pathologies, escape variants can emerge which the immune system is unable to contain.

TABLE 1
Summary Table of Sequences

| SEQ | | |
|---|---|---|
| ID NO | Description | Sequence |
| 1 | Human | ASTKGPSVFPLAPSSKSTSGGTAALGCLVKDYFPEPVTVSWNSGAL |
| | IgG1 | TSGVHTFPAVLQSSGLYSLSSVVTVPSSSLGTQTYICNVNHKPSNTK |
| | constant | VDKKVEPKSCDKTHTCPPCPAPELLGGPSVFLFPPKPKDTLMISRTP |
| | region | EVTCVVVDVSHEDPEVKFNWYVDGVEVHNAKTKPREEQYNSTYR |
| | (amino | VVSVLTVLHQDWLNGKEYKCKVSNKALPAPIEKTISKAKGQPREP |
| | acid | QVYTLPPSRDELTKNQVSLTCLVKGFYPSDIAVEWESNGQPENNYK |
| | sequence) | TTPPVLDSDGSFFLYSKLTVDKSRWQQGNVFSCSVMHEALHNHYT |
| | | QKSLSLSPGK |
| 2 | Human | EPKSCDKTHTCPPCPAPELLGGPSVFLFPPKPKDTLMISRTPEVTCV |
| | IgG1 Fc | VVDVSHEDPEVKFNWYVDGVEVHNAKTKPREEQYNSTYRVVSVL |
| | domain | TVLHQDWLNGKEYKCKVSNKALPAPIEKTISKAKGQPREPQVYTLP |
| | (amino | PSRDELTKNQVSLTCLVKGFYPSDIAVEWESNGQPENNYKTTPPVL |
| | acid | DSDGSFFLYSKLTVDKSRWQQGNVFSCSVMHEALHNHYTQKSLSL |
| | sequence) | SPGK |
| 3 | Mouse IL- | GCACCCACTTCAAGCTCCACTTCAAGCTCTACAGCGGAAGCACA |
| | 2 (nucleic | GCAGCAGCAGCAGCAGCAGCAGCAGCAGCAGCAGCACCTGGAG |
| | acid | CAGCTGTTGATGGACCTACAGGAGCTCCTGAGCAGGATGGAGA |
| | sequence) | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGAACTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAA |
| 4 | Mouse IL- | APTSSSTSSSTAEAQQQQQQQQQQQQHLEQLLMDLQELLSRMENY |
| | 2 (amino | RNLKLPRMLTFKFYLPKQATELKDLQCLEDELGPLRHVLDLTQSKS |
| | acid | FQLEDAENFISNIRVTVVKLKGSDNTFECQFDDESATVVDFLRRWI |
| | sequence) | AFCQSIISTSPQ |
| 5 | QQ6210 | GCACCCACTTCAAGCTCCACTTCAAGCTCTACAGCGGAAGCACA |
| | (nucleic | ACAGCAGCAGCAGCAGCAGCAGCAGCAGCAGCAGCACCTGGAG |
| | acid | CAGCTGTTGATGGACCTACAGGAACTCCTGAGTAGGATGGAGG |
| | sequence) | ATCACAGGAACCTGAGACTCCCCAGGATGCTCACCTTCAAATTT |
| | | TACTTGCCCGAGCAGGCCACAGAATTGGAAGATCTTCAGTGCCT |
| | | AGAAGATGAACTTGAACCACTGCGGCAAGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGACGATGAGCCAGCAACTGTGGTGGACT |
| | | TTCTGAGGAGATGGATAGCCTTCTGTCAAAGCATCATCTCAACA |
| | | AGCCCTCAA |
| 6 | QQ6210 | APTSSSTSSSTAEAQQQQQQQQQQQQHLEQLLMDLQELLSRMEDH |
| | (amino | RNLRLPRMLTFKFYLPEQATELEDLQCLEDELEPLRQVLDLTQSKSF |
| | acid | QLEDAENFISNIRVTVVKLKGSDNTFECQFDDEPATVVDFLRRWIA |
| | sequence) | FCQSIISTSPQ |
| 7 | E76A | GCACCCACTTCAAGCTCCACTTCAAGCTCTACAGCGGAAGCACA |
| | (nucleic | GCAGCAGCAGCAGCAGCAGCAGCAGCAGCAGCAGCACCTGGAG |
| | acid | CAGCTGTTGATGGACCTACAGGAGCTCCTGAGCAGGATGGAGA |
| | sequence) | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGCTCTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAA |
| 8 | E76A | APTSSSTSSSTAEAQQQQQQQQQQQQHLEQLLMDLQELLSRMENY |
| | (amino | RNLKLPRMLTFKFYLPKQATELKDLQCLEDALGPLRHVLDLTQSKS |
| | acid | FQLEDAENFISNIRVTVVKLKGSDNTFECQFDDESATVVDFLRRWI |
| | sequence) | AFCQSIISTSPQ |
| 9 | E76G | GCACCCACTTCAAGCTCCACTTCAAGCTCTACAGCGGAAGCACA |
| | (nucleic | GCAGCAGCAGCAGCAGCAGCAGCAGCAGCAGCAGCACCTGGAG |
| | acid | CAGCTGTTGATGGACCTACAGGAGCTCCTGAGCAGGATGGAGA |
| | sequence) | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGGTCTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAA |
| 10 | E76G | APTSSSTSSSTAEAQQQQQQQQQQQQHLEQLLMDLQELLSRMENY |
| | (amino | RNLKLPRMLTFKFYLPKQATELKDLQCLEDGLGPLRHVLDLTQSKS |
| | acid | FQLEDAENFISNIRVTVVKLKGSDNTFECQFDDESATVVDFLRRWI |
| | sequence) | AFCQSIISTSPQ |
| 11 | D265A | ATGAGGGTCCCCGCTCAGCTCCTGGGGCTCCTGCTGCTCTGGCT |
| | Fc/Flag | CCCAGGTGCACGATGTGAGCCCAGAGTGCCCATAACACAGAAC |
| | (nucleic | CCCTGTCCTCCACTCAAAGAGTGTCCCCCATGCGCAGCTCCAGA |
| | acid | CCTCTTGGGTGGACCATCCGTCTTCATCTTCCCTCCAAAGATCAA |
| | sequence) | GGATGTACTCATGATCTCCCTGAGCCCCATGGTCACATGTGTGG |
| | (C-terminal | TGGTGGCCGTGAGCGAGGATGACCCAGACGTCCAGATCAGCTG |
| | flag tag is | GTTTGTGAACAACGTGGAAGTACACACAGCTCAGACACAAACC |
| | underlined) | CATAGAGAGGATTACAACAGTACTCTCCGGGTGGTCAGTGCCCT |
| | | CCCCATCCAGCACCAGGACTGGATGAGTGGCAAGGAGTTCAAA |
| | | TGCAAGGTCAACAACAGAGCCCTCCCATCCCCCATCGAGAAAAC |
| | | CATCTCAAAACCCAGAGGGCCAGTAAGAGCTCCACAGGTATAT |
| | | GTCTTGCCTCCACCAGCAGAAGAGATGACTAAGAAAGAGTTCA |
| | | GTCTGACCTGCATGATCACAGGCTTCTTACCTGCCGAAATTGCT |
| | | GTGGACTGGACCAGCAATGGGCGTACAGAGCAAAACTACAAGA |
| | | ACACCGCAACAGTCCTGGACTCTGATGGTTCTTACTTCATGTAC |
| | | AGCAAGCTCAGAGTACAAAAGAGCACTTGGGAAAGAGGAAGTC |
| | | TTTTCGCCTGCTCAGTGGTCCACGAGGGTCTGCACAATCACCTTA |
| | | CGACTAAGACCATCTCCCGGTCTCTGGGTAAAGGTGGCGGATCT |
| | | GACTACAAGGACGACGATGACAAGTGATAA |
| 12 | D265A | MRVPAQLLGLLLLWLPGARCEPRVPITQNPCPPLKECPPCAAPDLL |
| | Fc/Flag | GGPSVFIFPPKIKDVLMISLSPMVTCVVVAVSEDDPDVQISWFVNNV |
| | (amino | EVHTAQTQTHREDYNSTLRVVSALPIQHQDWMSGKEFKCKVNNR |
| | acid | ALPSPIEKTISKPRGPVRAPQVYVLPPPAEEMTKKEFSLTCMITGFLP |
| | sequence) | AEIAVDWTSNGRTEQNYKNTATVLDSDGSYFMYSKLRVQKSTWE |
| | (C-terminal | RGSLFACSVVHEGLHNHLTTKTISRSLGKGGGSDYKDDDDK |
| | flag tag is | |
| | underlined) | |
| 13 | D265A | ATGAGGGTCCCCGCTCAGCTCCTGGGGCTCCTGCTGCTCTGGCT |
| | Fc/wt mIL- | CCCAGGTGCACGATGTGAGCCCAGAGTGCCCATAACACAGAAC |
| | 2 (nucleic | CCCTGTCCTCCACTCAAAGAGTGTCCCCCATGCGCAGCTCCAGA |
| | acid | CCTCTTGGGTGGACCATCCGTCTTCATCTTCCCTCCAAAGATCAA |
| | sequence) | GGATGTACTCATGATCTCCCTGAGCCCCATGGTCACATGTGTGG |
| | (C-terminal | TGGTGGCCGTGAGCGAGGATGACCCAGACGTCCAGATCAGCTG |
| | 6x his tag | GTTTGTGAACAACGTGGAAGTACACACAGCTCAGACACAAACC |
| | is | CATAGAGAGGATTACAACAGTACTCTCCGGGTGGTCAGTGCCCT |
| | underlined) | CCCCATCCAGCACCAGGACTGGATGAGTGGCAAGGAGTTCAAA |
| | | TGCAAGGTCAACAACAGAGCCCTCCCATCCCCCATCGAGAAAAC |
| | | CATCTCAAAACCCAGAGGGCCAGTAAGAGCTCCACAGGTATAT |
| | | GTCTTGCCTCCACCAGCAGAAGAGATGACTAAGAAAGAGTTCA |
| | | GTCTGACCTGCATGATCACAGGCTTCTTACCTGCCGAAATTGCT |
| | | GTGGACTGGACCAGCAATGGGCGTACAGAGCAAAACTACAAGA |
| | | ACACCGCAACAGTCCTGGACTCTGATGGTTCTTACTTCATGTAC |
| | | AGCAAGCTCAGAGTACAAAAGAGCACTTGGGAAAGAGGAAGTC |
| | | TTTTCGCCTGCTCAGTGGTCCACGAGGGTCTGCACAATCACCTTA |
| | | CGACTAAGACCATCTCCCGGTCTCTGGGTAAAGGAGGGGGCTCC |
| | | GCACCCACTTCAAGCTCCACTTCAAGCTCTACAGCGGAAGCACA |
| | | GCAGCAGCAGCAGCAGCAGCAGCAGCAGCAGCAGCACCTGGAG |
| | | CAGCTGTTGATGGACCTACAGGAGCTCCTGAGCAGGATGGAGA |
| | | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGAACTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAACACCATCACCACCATCACTGATAA |
| 14 | D265A | MRVPAQLLGLLLLWLPGARCEPRVPITQNPCPPLKECPPCAAPDLL |
| | Fc/wt mIL- | GGPSVFIFPPKIKDVLMISLSPMVTCVVVAVSEDDPDVQISWFVNNV |
| | 2 (amino | EVHTAQTQTHREDYNSTLRVVSALPIQHQDWMSGKEFKCKVNNR |
| | acid | ALPSPIEKTISKPRGPVRAPQVYVLPPPAEEMTKKEFSLTCMITGFLP |
| | sequence) | AEIAVDWTSNGRTEQNYKNTATVLDSDGSYFMYSKLRVQKSTWE |
| | (C- terminal | RGSLFACSVVHEGLHNHLTTKTISRSLGKGGGSAPTSSSTSSSTAEA |
| | 6x his tag | QQQQQQQQQQQQHLEQLLMDLQELLSRMENYRNLKLPRMLTFKF |
| | is | YLPKQATELKDLQCLEDELGPLRHVLDLTQSKSFQLEDAENFISNIR |
| | underlined) | VTVVKLKGSDNTFECQFDDESATVVDFLRRWIAFCQSIISTSPQHHH |
| | | HHH** |
| 15 | D265A Fc/ | ATGAGGGTCCCCGCTCAGCTCCTGGGGCTCCTGCTGCTCTGGCT |
| | QQ6210 | CCCAGGTGCACGATGTGAGCCCAGAGTGCCCATAACACAGAAC |
| | (nucleic | CCCTGTCCTCCACTCAAAGAGTGTCCCCCATGCGCAGCTCCAGA |
| | acid | CCTCTTGGGTGGACCATCCGTCTTCATCTTCCCTCCAAAGATCAA |
| | sequence) | GGATGTACTCATGATCTCCCTGAGCCCCATGGTCACATGTGTGG |
| | (C-terminal | TGGTGGCCGTGAGCGAGGATGACCCAGACGTCCAGATCAGCTG |
| | 6x his tag | GTTTGTGAACAACGTGGAAGTACACACAGCTCAGACACAAACC |
| | is | CATAGAGAGGATTACAACAGTACTCTCCGGGTGGTCAGTGCCCT |
| | underlined) | CCCCATCCAGCACCAGGACTGGATGAGTGGCAAGGAGTTCAAA |
| | | TGCAAGGTCAACAACAGAGCCCTCCCATCCCCCATCGAGAAAAC |
| | | CATCTCAAAACCCAGAGGGCCAGTAAGAGCTCCACAGGTATAT |
| | | GTCTTGCCTCCACCAGCAGAAGAGATGACTAAGAAAGAGTTCA |
| | | GTCTGACCTGCATGATCACAGGCTTCTTACCTGCCGAAATTGCT |
| | | GTGGACTGGACCAGCAATGGGCGTACAGAGCAAAACTACAAGA |
| | | ACACCGCAACAGTCCTGGACTCTGATGGTTCTTACTTCATGTAC |
| | | AGCAAGCTCAGAGTACAAAAGAGCACTTGGGAAAGAGGAAGTC |
| | | TTTTCGCCTGCTCAGTGGTCCACGAGGGTCTGCACAATCACCTTA |
| | | CGACTAAGACCATCTCCCGGTCTCTGGGTAAAGGAGGGGGCTCC |
| | | GCACCCACTTCAAGCTCCACTTCAAGCTCTACAGCGGAAGCACA |
| | | ACAGCAGCAGCAGCAGCAGCAGCAGCAGCAGCAGCACCTGGAG |
| | | CAGCTGTTGATGGACCTACAGGAACTCCTGAGTAGGATGGAGG |
| | | ATCACAGGAACCTGAGACTCCCCAGGATGCTCACCTTCAAATTT |
| | | TACTTGCCCGAGCAGGCCACAGAATTGGAAGATCTTCAGTGCCT |
| | | AGAAGATGAACTTGAACCACTGCGGCAAGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGACGATGAGCCAGCAACTGTGGTGGACT |
| | | TTCTGAGGAGATGGATAGCCTTCTGTCAAAGCATCATCTCAACA |
| | | AGCCCTCAACACCATCACCACCATCACTGATAA |
| 16 | D265A Fc/ | MRVPAQLLGLLLLWLPGARCEPRVPITQNPCPPLKECPPCAAPDLL |
| | QQ6210 | GGPSVFIFPPKIKDVLMISLSPMVTCVVVAVSEDDPDVQISWFVNNV |
| | (amino | EVHTAQTQTHREDYNSTLRVVSALPIQHQDWMSGKEFKCKVNNR |
| | acid | ALPSPIEKTISKPRGPVRAPQVYVLPPPAEEMTKKEFSLTCMITGFLP |
| | sequence) | AEIAVDWTSNGRTEQNYKNTATVLDSDGSYFMYSKLRVQKSTWE |
| | (C- terminal | RGSLFACSVVHEGLHNHLTTKTISRSLGKGGGSAPTSSSTSSSTAEA |
| | 6x his tag | QQQQQQQQQQQQHLEQLLMDLQELLSRMEDHRNLRLPRMLTFKF |
| | is | YLPEQATELEDLQCLEDELEPLRQVLDLTQSKSFQLEDAENFISNIR |
| | underlined) | VTVVKLKGSDNTFECQFDDEPATVVDFLRRWIAFCQSIISTSPQHHH |
| | | HHH |
| 17 | D265A Fc/ | ATGAGGGTCCCCGCTCAGCTCCTGGGGCTCCTGCTGCTCTGGCT |
| | E76A | CCCAGGTGCACGATGTGAGCCCAGAGTGCCCATAACACAGAAC |
| | (nucleic | CCCTGTCCTCCACTCAAAGAGTGTCCCCCATGCGCAGCTCCAGA |
| | acid | CCTCTTGGGTGGACCATCCGTCTTCATCTTCCCTCCAAAGATCAA |
| | sequence) | GGATGTACTCATGATCTCCCTGAGCCCCATGGTCACATGTGTGG |
| | (C-terminal | TGGTGGCCGTGAGCGAGGATGACCCAGACGTCCAGATCAGCTG |
| | 6x his tag | GTTTGTGAACAACGTGGAAGTACACACAGCTCAGACACAAACC |
| | is | CATAGAGAGGATTACAACAGTACTCTCCGGGTGGTCAGTGCCCT |
| | underlined) | CCCCATCCAGCACCAGGACTGGATGAGTGGCAAGGAGTTCAAA |
| | | TGCAAGGTCAACAACAGAGCCCTCCCATCCCCCATCGAGAAAAC |
| | | CATCTCAAAACCCAGAGGGCCAGTAAGAGCTCCACAGGTATAT |
| | | GTCTTGCCTCCACCAGCAGAAGAGATGACTAAGAAAGAGTTCA |
| | | GTCTGACCTGCATGATCACAGGCTTCTTACCTGCCGAAATTGCT |
| | | GTGGACTGGACCAGCAATGGGCGTACAGAGCAAAACTACAAGA |
| | | ACACCGCAACAGTCCTGGACTCTGATGGTTCTTACTTCATGTAC |
| | | AGCAAGCTCAGAGTACAAAAGAGCACTTGGGAAAGAGGAAGTC |
| | | TTTTCGCCTGCTCAGTGGTCCACGAGGGTCTGCACAATCACCTTA |
| | | CGACTAAGACCATCTCCCGGTCTCTGGGTAAAGGAGGGGGCTCC |
| | | GCACCCACTTCAAGCTCCACTTCAAGCTCTACAGCGGAAGCACA |
| | | GCAGCAGCAGCAGCAGCAGCAGCAGCAGCAGCAGCACCTGGAG |
| | | CAGCTGTTGATGGACCTACAGGAGCTCCTGAGCAGGATGGAGA |
| | | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGCTCTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAACACCATCACCACCATCACTGATAA |
| 18 | D265A Fc/ | MRVPAQLLGLLLLWLPGARCEPRVPITQNPCPPLKECPPCAAPDLL |
| | E76A | GGPSVFIFPPKIKDVLMISLSPMVTCVVVAVSEDDPDVQISWFVNNV |
| | (amino | EVHTAQTQTHREDYNSTLRVVSALPIQHQDWMSGKEFKCKVNNR |
| | acid | ALPSPIEKTISKPRGPVRAPQVYVLPPPAEEMTKKEFSLTCMITGFLP |
| | sequence) | AEIAVDWTSNGRTEQNYKNTATVLDSDGSYFMYSKLRVQKSTWE |
| | (C-terminal | RGSLFACSVVHEGLHNHLTTKTISRSLGKGGGSAPTSSSTSSSTAEA |
| | 6x his tag | QQQQQQQQQQQQHLEQLLMDLQELLSRMENYRNLKLPRMLTFKF |
| | is | YLPKQATELKDLQCLEDALGPLRHVLDLTQSKSFQLEDAENFISNIR |
| | underlined) | VTVVKLKGSDNTFECQFDDESATVVDFLRRWIAFCQSIISTSPQHHH |
| | | HHH |
| 19 | D265A Fc/ | ATGAGGGTCCCCGCTCAGCTCCTGGGGCTCCTGCTGCTCTGGCT |
| | E76G | CCCAGGTGCACGATGTGAGCCCAGAGTGCCCATAACACAGAAC |
| | (nucleic | CCCTGTCCTCCACTCAAAGAGTGTCCCCCATGCGCAGCTCCAGA |
| | acid | CCTCTTGGGTGGACCATCCGTCTTCATCTTCCCTCCAAAGATCAA |
| | sequence) | GGATGTACTCATGATCTCCCTGAGCCCCATGGTCACATGTGTGG |
| | (C-terminal | TGGTGGCCGTGAGCGAGGATGACCCAGACGTCCAGATCAGCTG |
| | 6x his tag | GTTTGTGAACAACGTGGAAGTACACACAGCTCAGACACAAACC |
| | is | CATAGAGAGGATTACAACAGTACTCTCCGGGTGGTCAGTGCCCT |
| | underlined) | CCCCATCCAGCACCAGGACTGGATGAGTGGCAAGGAGTTCAAA |
| | | TGCAAGGTCAACAACAGAGCCCTCCCATCCCCCATCGAGAAAAC |
| | | CATCTCAAAACCCAGAGGGCCAGTAAGAGCTCCACAGGTATAT |
| | | GTCTTGCCTCCACCAGCAGAAGAGATGACTAAGAAAGAGTTCA |
| | | GTCTGACCTGCATGATCACAGGCTTCTTACCTGCCGAAATTGCT |
| | | GTGGACTGGACCAGCAATGGGCGTACAGAGCAAAACTACAAGA |
| | | ACACCGCAACAGTCCTGGACTCTGATGGTTCTTACTTCATGTAC |
| | | AGCAAGCTCAGAGTACAAAAGAGCACTTGGGAAAGAGGAAGTC |
| | | TTTTCGCCTGCTCAGTGGTCCACGAGGGTCTGCACAATCACCTTA |
| | | CGACTAAGACCATCTCCCGGTCTCTGGGTAAAGGAGGGGGCTCC |
| | | GCACCCACTTCAAGCTCCACTTCAAGCTCTACAGCGGAAGCACA |
| | | GCAGCAGCAGCAGCAGCAGCAGCAGCAGCAGCAGCACCTGGAG |
| | | CAGCTGTTGATGGACCTACAGGAGCTCCTGAGCAGGATGGAGA |
| | | ATTACAGGAACCTGAAACTCCCCAGGATGCTCACCTTCAAATTT |
| | | TACTTGCCCAAGCAGGCCACAGAATTGAAAGATCTTCAGTGCCT |
| | | AGAAGATGGTCTTGGACCTCTGCGGCATGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGTCAGCAACTGTGGTGGACT |
| | | TTCTGAGGAGATGGATAGCCTTCTGTCAAAGCATCATCTCAACA |
| | | AGCCCTCAACACCATCACCACCATCACTGATAA |
| 20 | D265A Fc/ | MRVPAQLLGLLLLWLPGARCEPRVPITQNPCPPLKECPPCAAPDLL |
| | E76G | GGPSVFIFPPKIKDVLMISLSPMVTCVVVAVSEDDPDVQISWFVNNV |
| | (amino | EVHTAQTQTHREDYNSTLRVVSALPIQHQDWMSGKEFKCKVNNR |
| | acid | ALPSPIEKTISKPRGPVRAPQVYVLPPPAEEMTKKEFSLTCMITGFLP |
| | sequence) | AEIAVDWTSNGRTEQNYKNTATVLDSDGSYFMYSKLRVQKSTWE |
| | (C-terminal | RGSLFACSVVHEGLHNHLTTKTISRSLGKGGGSAPTSSSTSSSTAEA |
| | 6x his tag | QQQQQQQQQQQQHLEQLLMDLQELLSRMENYRNLKLPRMLTFKF |
| | is | YLPKQATELKDLQCLEDGLGPLRHVLDLTQSKSFQLEDAENFISNIR |
| | underlined) | VTVVKLKGSDNTFECQFDDESATVVDFLRRWIAFCQSIISTSPQHHH |
| | | HHH |
| 21 | mIL-2 QQ | GCACCCACCTCAAGCTCCACTTCAAGCTCTACAGCGGAAGCACA |
| | 6.2-4 | ACAGCAGCAGCAGCAGCAGCAGCAGCAGCAGCAGCACCTGGAG |
| | (nucleic | CAGCTGTTGATGGACCTACAGGAGCTCCTGAGCAGGATGGAGG |
| | acid | ATTCCAGGAACCTGAGACTCCCCAGGATGCTCACCTTCAAATTT |
| | sequence) | TACTTGCCCAAGCAGGCCACAGAATTGGAAGATCTTCAGTGCCT |
| | | AGAAGATGAACTTGAACCTCTGCGGCAAGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGCCAGCAACTGTGGTGGGCT |
| | | TTCTGAGGAGATGGATAGCCTTCTGTCAAAGCATCATCTCAACG |
| | | AGCCCTCAA |
| 22 | mIL-2 QQ | APTSSSTSSSTAEAQQQQQQQQQQQQHLEQLLMDLQELLSRMEDS |
| | 6.2-4 | RNLRLPRMLTFKFYLPKQATELEDLQCLEDELEPLRQVLDLTQSKS |
| | (amino | FQLEDAENFISNIRVTVVKLKGSDNTFECQFDDEPATVVGFLRRWI |
| | acid | AFCQSIISTSPQ |
| | sequence) | |
| 23 | mIL-2 QQ | GCACCCACCTCAAGCTCCACTTCAAGCTCTACAGCGGAAGCACA |
| | 6.2-8 | ACAGCAGCAGCAGCAGCAGCAGCACCTGGAGCAGCTGTTGATG |
| | (nucleic | GACCTACAGGAGCTCCTGAGTAGGATGGAGGATCACAGGAACC |
| | acid | TGAGACTCCCCAGGATGCTCACCTTCAAATTTTACTTGCCCAAG |
| | sequence) | CAGGCCACAGAATTGGAAGATCTTCAGTGCCTAGAAGATGAACT |
| | | TGAACCTCTGCGGCAAGTTCTGGATTTGACTCAAAGCAAAAGCT |
| | | TTCAATTGGAAGATGCTGAGAATTTCATCAGCAATATCAGAGTA |
| | | ACTGTTGTAAAACTAAAGGGCTCTGACAACACATTTGAGTGCCA |
| | | ATTCGATGATGAGCCAGCAACTGTGGTGGACTTTCTGAGGAGAT |
| | | GGATAGCCTTCTGTCAAAGCATCATCTCAACAAGCCCTCGA |
| 24 | mIL-2 QQ | APTSSSTSSSTAEAQQQQQQQQHLEQLLMDLQELLSRMEDHRNLR |
| | 6.2-8 | LPRMLTFKFYLPKQATELEDLQCLEDELEPLRQVLDLTQSKSFQLE |
| | (amino | DAENFISNIRVTVVKLKGSDNTFECQFDDEPATVVDFLRRWIAFCQ |
| | acid | SIISTSPR |
| | sequence) | |
| 25 | mIL-2 QQ | GCACCCACCTCAAGCTCCACTTCAAGCTCTACAGCGGAAGCACA |
| | 6.2-10 | ACAGCAGCAGCAGCAGCAGCAGCAGCAGCAGCAGCACCTGGAG |
| | (nucleic | CAGCTGTTGATGGACCTACAGGAACTCCTGAGTAGGATGGAGG |
| | acid | ATCACAGGAACCTGAGACTCCCCAGGATGCTCACCTTCAAATTT |
| | sequence) | TACTTGCCCGAGCAGGCCACAGAATTGGAAGATCTTCAGTGCCT |
| | | AGAAGATGAACTTGAACCACTGCGGCAAGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGACGATGAGCCAGCAACTGTGGTGGACT |
| | | TTCTGAGGAGATGGATAGCCTTCTGTCAAAGCATCATCTCAACA |
| | | AGCCCTCAG |
| 26 | mIL-2 QQ | APTSSSTSSSTAEAQQQQQQQQQQQQHLEQLLMDLQELLSRMEDH |
| | 6.2-10 | RNLRLPRMLTFKFYLPEQATELEDLQCLEDELEPLRQVLDLTQSKSF |
| | (amino | QLEDAENFISNIRVTVVKLKGSDNTFECQFDDEPATVVDFLRRWIA |
| | acid | FCQSIISTSPQ |
| | sequence) | |
| 27 | mIL-2 QQ | GCACCCACCTCAAGCTCCACTTCAAGCTCTACAGCGGAAGCACA |
| | 6.2-11 | ACAGCAGCAGCAGCAGCAGCAGCAGCACCTGGAGCAGCTGTTG |
| | (nucleic | ATGGACCTACAGGAGCTCCTGAGCAGGATGGAGGATTCCAGGA |
| | acid | ACCTGAGACTCCCCAGAATGCTCACCTTCAAATTTTACTTGCCCG |
| | sequence) | AGCAGGCCACAGAATTGAAAGATCTCCAGTGCCTAGAAGATGA |
| | | ACTTGAACCTCTGCGGCAAGTTCTGGATTTGACTCAAAGCAAAA |
| | | GCTTTCAATTGGAAGATGCTGAGAATTTCATCAGCAATATCAGA |
| | | GTAACTGTTGTAAAACTAAAGGGCTCTGACAACACATTTGAGTG |
| | | CCAATTCGACGATGAGCCAGCAACTGTGGTGGACTTTCTGAGGA |
| | | GATGGATAGCCTTCTGTCAAAGCATCATCTCAACAAGCCCTCAG |
| 28 | mIL-2 QQ | APTSSSTSSSTAEAQQQQQQQQQHLEQLLMDLQELLSRMEDSRNL |
| | 6.2-11 | RLPRMLTFKFYLPEQATELKDLQCLEDELEPLRQVLDLTQSKSFQL |
| | (amino | EDAENFISNIRVTVVKLKGSDNTFECQFDDEPATVVDFLRRWIAFC |
| | acid | QSIISTSPQ |
| | sequence) | |
| 29 | mIL-2 QQ | GCACCCACCTCAAGCTCCACTTCAAGCTCTACAGCGGAAGCACA |
| | 6.2-13 | ACAGCAGCAGCAGCAGCAGCAGCAGCAGCAGCAGCACCTGGAG |
| | (nucleic | CAGCTGTTGATGGACCTACAGGAGCTCCTGAGTAGGATGGAGG |
| | acid | ATCACAGGAACCTGAGACTCCCCAGGATGCTCACCTTCAAATTT |
| | sequence) | TACTTGCCCGAGCAGGCCACAGAATTGAAAGATCTCCAGTGCCT |
| | | AGAAGATGAACTTGAACCTCTGCGGCAGGTTCTGGATTTGACTC |
| | | AAAGCAAAAGCTTTCAATTGGAAGATGCTGAGAATTTCATCAGC |
| | | AATATCAGAGTAACTGTTGTAAAACTAAAGGGCTCTGACAACAC |
| | | ATTTGAGTGCCAATTCGATGATGAGCCAGCAACTGTGGTGGACT |
| | | TTCTGAGGAGATGGATAGCCTTCTGTCAAAGCATCATCTCAACA |
| | | AGCCCTCAG |
| 30 | mIL-2 QQ | APTSSSTSSSTAEAQQQQQQQQQQQQHLEQLLMDLQELLSRMEDH |
| | 6.2-13 | RNLRLPRMLTFKFYLPEQATELKDLQCLEDELEPLRQVLDLTQSKS |
| | (amino | FQLEDAENFISNIRVTVVKLKGSDNTFECQFDDEPATVVDFLRRWI |
| | acid | AFCQSIISTSPQ |
| | sequence) | |
| 31 | Full length | ATGTACAGGATGCAACTCCTGTCTTGCATTGCACTAAGTCTTGC |
| | human IL- | ACTTGTCACAAACAGTGCACCTACTTCAAGTTCTACAAAGAAAA |
| | 2 (nucleic | CACAGCTACAACTGGAGCATTTACTGCTGGATTTACAGATGATT |
| | acid | TTGAATGGAATTAATAATTACAAGAATCCCAAACTCACCAGGAT |
| | sequence) | GCTCACATTTAAGTTTTACATGCCCAAGAAGGCCACAGAACTGA |
| | | AACATCTTCAGTGTCTAGAAGAAGAACTCAAACCTCTGGAGGAA |
| | | GTGCTAAATTTAGCTCAAAGCAAAAACTTTCACTTAAGACCCAG |
| | | GGACTTAATCAGCAATATCAACGTAATAGTTCTGGAACTAAAGG |
| | | GATCTGAAACAACATTCATGTGTAATATGCTGATGAGACAGCAA |
| | | CCATTGTAGAATTTCTGAACAGATGGATTACCTTTTGTCAAAGC |
| | | ATCATCTCAACACTGACTTGA |
| 32 | Full length | MYRMQLLSCIALSLALVTNSAPTSSSTKKTQLQLEHLLLDLQMILN |
| | human IL- | GINNYKNPKLTRMLTFKFYMPKKATELKHLQCLEEELKPLEEVLNL |
| | 2 (amino | AQSKNFHLRPRDLISNINVIVLELKGSETTFMCEYADETATIVEFLN |
| | acid | RWITFCQSIISTLT |
| | sequence) | |
| 33 | Human IL- | GCACCTACTTCAAGTTCTACAAAGAAAACACAGCTACAACTGGA |
| | 2 without | GCATTTACTGCTGGATTTACAGATGATTTTGAATGGAATTAATA |
| | signal | ATTACAAGAATCCCAAACTCACCAGGATGCTCACATTTAAGTTT |
| | peptide | TACATGCCCAAGAAGGCCACAGAACTGAAACATCTTCAGTGTCT |
| | (nucleic | AGAAGAAGAACTCAAACCTCTGGAGGAAGTGCTAAATTTAGCT |
| | acid | CAAAGCAAAAACTTTCACTTAAGACCCAGGGACTTAATCAGCAA |
| | sequence) | TATCAACGTAATAGTTCTGGAACTAAAGGGATCTGAAACAACAT |
| | | TCATGTGTAATATGCTGATGAGACAGCAACCATTGTAGAATTTC |
| | | TGAACAGATGGATTACCTTTTGTCAAAGCATCATCTCAACACTG |
| | | ACTTGA |
| 34 | Human IL- | APTSSSTKKTQLQLEHLLLDLQMILNGINNYKNPKLTRMLTFKFYM |
| | 2 without | PKKATELKHLQCLEEELKPLEEVLNLAQSKNFHLRPRDLISNINVIV |
| | signal | LELKGSETTFMCEYADETATIVEFLNRWITFCQSIISTLT |
| | peptide | |
| | (amino | |
| | acid | |
| | sequence) | |
| 35 | Human | MDMRVPAQLLGLLLLWLPGARCADAHKSEVAHRFKDLGEENFKA |
| | serum | LVLIAFAQYLQQCPFEDHVKLVNEVTEFAKTCVADESAENCDKSL |
| | albumin | HTLFGDKLCTVATLRETYGEMADCCAKQEPERNECFLQHKDDNPN |
| | (amino | LPRLVRPEVDVMCTAFHDNEETFLKKYLYEIARRHPYFYAPELLFF |
| | acid | AKRYKAAFTECCQAADKAACLLPKLDELRDEGKASSAKQRLKCAS |
| | sequence) | LQKFGERAFKAWAVARLSQRFPKAEFAEVSKLVTDLTKVHTECCH |
| | | GDLLECADDRADLAKYICENQDSISSKLKECCEKPLLEKSHCIAEVE |
| | | NDEMPADLPSLAADFVESKDVCKNYAEAKDVFLGMFLYEYARRH |
| | | PDYSVVLLLRLAKTYETTLEKCCAAADPHECYAKVFDEFKPLVEEP |
| | | QNLIKQNCELFEQLGEYKFQNALLVRYTKKVPQVSTPTLVEVSRNL |
| | | GKVGSKCCKHPEAKRMPCAEDYLSVVLNQLCVLHEKTPVSDRVT |
| | | KCCTESLVNRRPCFSALEVDETYVPKEFNAETFTFHADICTLSEKER |
| | | QIKKQTALVELVKHKPKATKEQLKAVMDDFAAFVEKCCKADDKE |
| | | TCFAEEGKKLVAASQAALGLGGGSAPTSSSTKKTQLQLEHLLLDLQ |
| | | MILNGINNYKNPKLTRMLTFKFYMPKKATELKHLQCLEEELKPLEE |
| | | VLNLAQSKNFHLRPRDLISNINVIVLELKGSETTFMCEYADETATIV |
| | | EFLNRWITFCQSIISTLTGGGS |
| 36 | Mature | DAHKSEVAHRFKDLGEENFKALVLIAFAQYLQQCPFEDHVKLVNE |
| | HSA | VTEFAKTCVADESAENCDKSLHTLFGDKLCTVATLRETYGEMADC |
| | (amino | CAKQEPERNECFLQHKDDNPNLPRLVRPEVDVMCTAFHDNEETFL |
| | acid | KKYLYEIARRHPYFYAPELLFFAKRYKAAFTECCQAADKAACLLP |
| | sequence) | KLDELRDEGKASSAKQRLKCASLQKFGERAFKAWAVARLSQRFPK |
| | | AEFAEVSKLVTDLTKVHTECCHGDLLECADDRADLAKYICENQDSI |
| | | SSKLKECCEKPLLEKSHCIAEVENDEMPADLPSLAADFVESKDVCK |
| | | NYAEAKDVFLGMFLYEYARRHPDYSVVLLLRLAKTYETTLEKCCA |
| | | AADPHECYAKVFDEFKPLVEEPQNLIKQNCELFEQLGEYKFQNALL |
| | | VRYTKKVPQVSTPTLVEVSRNLGKVGSKCCKHPEAKRMPCAEDYL |
| | | SVVLNQLCVLHEKTPVSDRVTKCCTESLVNRRPCFSALEVDETYVP |
| | | KEFNAETFTFHADICTLSEKERQIKKQTALVELVKHKPKATKEQLK |
| | | AVMDDFAAFVEKCCKADDKETCFAEEGKKLVAASQAALGLGGGS |
| | | APTSSSTKKTQLQLEHLLLDLQMILNGINNYKNPKLTRMLTFKFYM |
| | | PKKATELKHLQCLEEELKPLEEVLNLAQSKNFHLRPRDLISNINVIV |
| | | LELKGSETTFMCEYADETATIVEFLNRWITFCQSIISTLTGGGS |
| 37 | Human | ATGGATATGCGGGTGCCTGCTCAGCTGCTGGGACTGCTGCTGCT |
| | serum | GTGGCTGCCTGGGGCTAGATGCGCCGATGCTCACAAAAGCGAA |
| | albumin | GTCGCACACAGGTTCAAAGATCTGGGGGAGGAAAACTTTAAGG |
| | (nucleic | CTCTGGTGCTGATTGCATTCGCCCAGTACCTGCAGCAGTGCCCCT |
| | acid | TTGAGGACCACGTGAAACTGGTCAACGAAGTGACTGAGTTCGCC |
| | sequence) | AAGACCTGCGTGGCCGACGAATCTGCTGAGAATTGTGATAAAA |
| | | GTCTGCATACTCTGTTTGGGGATAAGCTGTGTACAGTGGCCACT |
| | | CTGCGAGAAACCTATGGAGAGATGGCAGACTGCTGTGCCAAAC |
| | | AGGAACCCGAGCGGAACGAATGCTTCCTGCAGCATAAGGACGA |
| | | TAACCCCAATCTGCCTCGCCTGGTGCGACCTGAGGTGGACGTCA |
| | | TGTGTACAGCCTTCCACGATAATGAGGAAACTTTTCTGAAGAAA |
| | | TACCTGTACGAAATCGCTCGGAGACATCCTTACTTTTATGCACC |
| | | AGAGCTGCTGTTCTTTGCCAAACGCTACAAGGCCGCTTTCACCG |
| | | AGTGCTGTCAGGCAGCCGATAAAGCTGCATGCCTGCTGCCTAAG |
| | | CTGGACGAACTGAGGGATGAGGGCAAGGCCAGCTCCGCTAAAC |
| | | AGCGCCTGAAGTGTGCTAGCCTGCAGAAATTCGGGGAGCGAGC |
| | | CTTCAAGGCTTGGGCAGTGGCACGGCTGAGTCAGAGATTCCCAA |
| | | AGGCAGAATTTGCCGAGGTCTCAAAACTGGTGACCGACCTGACA |
| | | AAGGTGCACACCGAATGCTGTCATGGCGACCTGCTGGAGTGCGC |
| | | CGACGATCGAGCTGATCTGGCAAAGTATATTTGTGAGAACCAGG |
| | | ACTCCATCTCTAGTAAGCTGAAAGAATGCTGTGAGAAACCACTG |
| | | CTGGAAAAGTCTCACTGCATTGCCGAAGTGGAGAACGACGAGA |
| | | TGCCAGCTGATCTGCCCTCACTGGCCGCTGACTTCGTCGAAAGC |
| | | AAAGATGTGTGTAAGAATTACGCTGAGGCAAAGGATGTGTTCCT |
| | | GGGAATGTTTCTGTACGAGTATGCCAGGCGCCACCCAGACTACT |
| | | CCGTGGTCCTGCTGCTGAGGCTGGCTAAAACATATGAAACCACA |
| | | CTGGAGAAGTGCTGTGCAGCCGCTGATCCCCATGAATGCTATGC |
| | | CAAAGTCTTCGACGAGTTTAAGCCCCTGGTGGAGGAACCTCAGA |
| | | ACCTGATCAAACAGAATTGTGAACTGTTTGAGCAGCTGGGCGAG |
| | | TACAAGTTCCAGAACGCCCTGCTGGTGCGCTATACCAAGAAAGT |
| | | CCCACAGGTGTCCACACCCACTCTGGTGGAGGTGAGCCGGAATC |
| | | TGGGCAAAGTGGGGAGTAAATGCTGTAAGCACCCTGAAGCCAA |
| | | GAGGATGCCATGCGCTGAGGATTACCTGAGTGTGGTCCTGAATC |
| | | AGCTGTGTGTCCTGCATGAAAAAACACCTGTCAGCGACCGGGTG |
| | | ACAAAGTGCTGTACTGAGTCACTGGTGAACCGACGGCCCTGCTT |
| | | TAGCGCCCTGGAAGTCGATGAGACTTATGTGCCTAAAGAGTTCA |
| | | ACGCTGAGACCTTCACATTTCACGCAGACATTTGTACCCTGAGC |
| | | GAAAAGGAGAGACAGATCAAGAAACAGACAGCCCTGGTCGAAC |
| | | TGGTGAAGCATAAACCCAAGGCCACAAAAGAGCAGCTGAAGGC |
| | | TGTCATGGACGATTTCGCAGCCTTTGTGGAAAAATGCTGTAAGG |
| | | CAGACGATAAGGAGACTTGCTTTGCCGAGGAAGGAAAGAAACT |
| | | GGTGGCTGCATCCCAGGCAGCTCTGGGACTGGGAGGAGGATCT |
| | | GCCCCTACCTCAAGCTCCACTAAGAAAACCCAGCTGCAGCTGGA |
| | | GCACCTGCTGCTGGACCTGCAGATGATTCTGAACGGGATCAACA |
| | | ATTACAAAAATCCAAAGCTGACCCGGATGCTGACATTCAAGTTT |
| | | TATATGCCCAAGAAAGCCACAGAGCTGAAACACCTGCAGTGCCT |
| | | GGAGGAAGAGCTGAAGCCTCTGGAAGAGGTGCTGAACCTGGCC |
| | | CAGAGCAAGAATTTCCATCTGAGACCAAGGGATCTGATCTCCAA |
| | | CATTAATGTGATCGTCCTGGAACTGAAGGGATCTGAGACTACCT |
| | | TTATGTGCGAATACGCTGACGAGACTGCAACCATTGTGGAGTTC |
| | | CTGAACAGATGGATCACCTTCTGCCAGTCCATCATTTCTACTCTG |
| | | ACAGGCGGGGGGAGC |
| 38 | PD-1 | MQIPQAPWPVVWAVLQLGWRPGWFLDSPDPWNPPTFFPALLVVTE |
| | | GDNATFTCSFSNTSESFVLNWYRMSPSNQTDKLAAFPEDRSQPGQD |
| | | CRFRVTQLPNGRDFHMSVVRARRNDSGTYLCGAISLAPKAQIKESL |
| | | RAELRVTERRAEVPTAHPSPSPRPAGQFQTLVVGVVGGLLGSLVLL |
| | | VWVLAVICSRAARGTIGARRTGQPLKEDPSAVPVFSVDYGELDFQ |
| | | WREKTPEPPVPCVPEQTEYATIVFPSGMGTSSPARRGSADGPRSAQP |
| | | LRPEDGHCSWPL |
| 39 | PD-L-1 | MRIFAVFIFMTYWHLLNAFTVTVPKDLYVVEYGSNMTIECKFPVEK |
| | | QLDLAALIVYWEMEDKNIIQFVHGEEDLKVQHSSYRQRARLLKDQ |
| | | LSLGNAALQITDVKLQDAGVYRCMISYGGADYKRITVKVNAPYNK |
| | | INQRILVVDPVTSEHELTCQAEGYPKAEVIWTSSDHQVLSGKTTTT |
| | | NSKREEKLFNVTSTLRINTTTNEIFYCTFRRLDPEENHTAELVIPELP |
| | | LAHPPNERTHLVILGAILLCLGVALTFIFRLRKGRMMDVKKCGIQD |
| | | TNSKKQSDTHLEET |
| 40 | CTLA-4 | MACLGFQRHKAQLNLATRTWPCTLLFFLLFIPVFCKAMHVAQPAV |
| | | VLASSRGIASFVCEYASPGKATEVRVTVLRQADSQVTEVCAATYM |
| | | MGNELTFLDDSICTGTSSGNQVNLTIQGLRAMDTGLYICKVELMYP |
| | | PPYYLGIGNGTQIYVIDPEPCPDSDFLLWILAAVSSGLFFYSFL |
| | | LTAVSLSKMLKKRSPLTTGVYVKMPPTEPECEKQFQPYFIPIN |
| 41 | LAG3 | MWEAQFLGLLFLQPLWVAPVKPLQPGAEVPVVWAQEGAPAQLPC |
| | | SPTIPLQDLSLLRRAGVTWQHQPDSGPPAAAPGHPLAPGPHPAAPSS |
| | | WGPRPRRYTVLSVGPGGLRSGRLPLQPRVQLDERGRQRGDFSLWL |
| | | RPARRADAGEYRAAVHLRDRALSCRLRLRLGQASMTASPPGSLR |
| | | ASDWVILNCSFSRPDRPASVHWFRNRGQGRVPVRESPHHHLAESFL |
| | | FLPQVSPMDSGPWGCILTYRDGFNVSIMYNLTVLGLEPPTPLTVYA |
| | | GAGSRVGLPCRLPAGVGTRSFLTAKWTPPGGGPDLLVTGDNGDFT |
| | | LRLEDVSQAQAGTYTCHIHLQEQQLNATVTLAIITVTPKSFGS |
| | | PGSLGKLLCEVTPVSGQERFVWSSLDTPSQRSFSGPWLEAQEAQLL |
| | | SQPWQCQLYQGERLLGAAVYFTELSSPGAQRSGRAPGALPAGHL |
| | | LLFLILGVLSLLLLVTGAFGFHLWRRQWRPRRFSALEQGI |
| | | HPPQAQSKIEELEQEPEPEPEPEPEPEPEPEPEQL |
| 42 | TIM3 | MFSHLPFDCVLLLLLLLLTRSSEVEYRAEVGQNAYLPCFYTPAAPG |
| | | NLVPVCWGKGACPVFECGNVVLRTDERDVNYWTSRYWLNGDFR |
| | | KGDVSLTIENVTLADSGIYCCRIQIPGIMNDEKFNLKLVIKPAKVTP |
| | | APTRQRDFTAAFPRMLTTRGHGPAETQTLGSLPDINLTQISTLA |
| | | NELRDSRLANDLRDSGATIRGIYIGAGICAGLALALIFGALIFKWYS |
| | | HSKEKIQNLSLISLANLPPSGLANAVAEGIRSEENIYTIEENVYEVEE |
| | | PNEYYCYVSSRQQPSQPLGCRFAMP |
| 43 | B7-H3 | MLRRRGSPGMGVHVGAALGALWFCLTGALEVQVPEDPVVALVGT |
| | | DATLCCSFSPEPGFSLQLNLIWQLTDTKQLVHSFAEGQDQGSAYA |
| | | NRTALFPDLLAQGNASLRLQRVRVADEGSFCFVSIRDFGSAAVSLQ |
| | | VAAPYSKPSMTLEPNKDLRPGDTVTITCSSYQGYPEAEVFWQD |
| | | GQGVPLTGNVTTSQMANEQGLFDVHSILRVVLGANGTYSCLVRNP |
| | | VLQQDAHSSVTITPQRSPTGAVEVQVPEDPVVALVGTDATLRCSF |
| | | SPEPGFSLAQLNLIWQLTDTKQLVHSFTEGRDQGSAYANRTALFPD |
| | | LLAQGNASLRLQRVRVADEGSFTCFVSIRDFGSAAVSLQVAAPY |
| | | SKPSMTLEPNKDLRPGDTVTITCSSYRGYPEAEVFWQDGQGVPLTG |
| | | NVTTSQMANEQGLFDVHSVLRVVLGANGTYSCLVRNPVLQQDAH |
| | | GSVTITGQPMTFPPEALWVTVGLSVCLIALLVALAFVCWRKIKQSC |
| | | EEENAGAEDQDGEGEGSKTALQPLKHSDSKEDDGQEIA |
| 44 | B7-H4 | MASLGQILFWSIISIIIILAGAIALIIGFGISAFSMPEVNVDYNASSETL |
| | | RCEAPRWFPQPTVVWASQVDQGANFSEVSNTSFELNSENVTMKVV |
| | | SVLYNVTINNTYSCMIENDIAKATGDIKVTESEIKRRSHLQLLNS |
| | | KASLCVSSFFAISWALLPLSPYLMLK |

Equivalents

Those skilled in the art will recognize or be able to ascertain, using no more than routine experimentation, many equivalents of the specific embodiments described herein described herein. Such equivalents are intended to be encompassed by the following claims.

We claim:

1. A method for treating a hyperproliferative disorder in a subject comprising administering to the subject a therapeutically effective amount of:
a. interleukin (IL)-2;
b. a therapeutic antibody or antibody fragment; and
c. a cancer vaccine.

2. The method of claim 1, wherein the IL-2 is an extended pharmacokinetic (PK) IL-2.

3. The method of claim 2, wherein the extended-PK IL-2 comprises a fusion protein.

4. The method of claim 3, wherein the fusion protein comprises an IL-2 moiety and a moiety selected from the group consisting of an immunoglobulin fragment, serum albumin, transferrin, and Fn3, or variants thereof.

5. The method of claim 1 or 2, wherein the IL-2 or extended-PK IL-2 comprises an IL-2 moiety conjugated to a non-protein polymer.

6. The method of claim 5, wherein the non-protein polymer is polyethylene glycol.

7. The method of claim 4, wherein the fusion protein comprises an IL-2 moiety operably linked to an immunoglobulin Fc domain.

8. The method of claim 4, wherein the fusion protein comprises an IL-2 moiety operably linked to human serum albumin.

9. The method of any one of claims 1-8, wherein the therapeutic antibody or antibody fragment recognizes a tumor antigen.

10. The method of claim 1, wherein the cancer vaccine is a population of cells immunized in vitro with a tumor antigen and administered to the subject.

11. The method of claim 1, wherein the cancer vaccine is an amphiphilic peptide conjugate comprising a tumor-associated antigen, and a lipid component, and optionally a linker, wherein the amphiphilic peptide conjugate binds albumin under physiological conditions.

12. The method of claim 11, wherein the tumor-associated antigen is conjugated to a lipid via a linker.

13. The method of claim 12, wherein the linker is selected from the group consisting of hydrophilic polymers, a string of hydrophilic amino acids, polysaccharides or a combination thereof.

14. The method of claim 13, wherein the linker comprises “N” consecutive polyethylene glycol units, wherein N is between 25-50.

15. The method of claim 11, wherein the lipid is a diacyl lipid.

16. The method of claim 11, wherein the cancer vaccine further comprises an adjuvant.

17. The method of claim 16, wherein the adjuvant is an amphiphilic oligonucloetide conjugate comprising an immunostimulatory oligonucelotide conjugated to a lipid with or without a linker, and optionally a polar compound, wherein the conjugate binds albumin under physiological conditions.

18. The method of claim 17, wherein the molecular adjuvant is an immunostimulatory oligonucleotide that can bind a pattern recognition receptor.

19. The method of claim 17, wherein the immunostimulatory oligonucleotide comprises CpG.

20. The method of claim 17, wherein the immunostimulatory oligonucelotide is a ligand for a toll-like receptor.

21. The method of claim 17, wherein the linker is an oligonucleotide linker.

22. The method of claim 18, wherein the oligonucelotide linker comprises “N” consecutive guanines, wherein N is between 0-2.

23. The method of claim 17, wherein the lipid is a diacyl lipid.

24. The method of any one of claims 1-23, further comprising administering an immune checkpoint blocker.

25. The method of claim 24, wherein the immune checkpoint blocker activates an anti-tumor immune response.

26. The method of claim 24, wherein the immune checkpoint blocker induces an increase in T cell proliferation, enhances T cell activation, and/or increases cytokine production (e.g., IFN-γ, IL-2).

27. The method of claim 26, wherein the immune checkpoint blocker targets the interaction between PD-1 and PD-L1, CTLA-4 CD80 or CD86, LAG3 and MHC class II molecules, TIM3 and galectin 9.

28. The method of claim 27, wherein the immune checkpoint blocker targets the interaction between PD-1 and PD-L1.

29. The method of claim 27, wherein the immune checkpoint blocker targets the interaction between CTLA-4 and CD80 or CD86.

30. The method of claim 27, wherein the immune checkpoint blocker targets the interaction between LAG3 and MHC class II molecules.

31. The method of claim 27, wherein the immune checkpoint blocker targets the interaction between TIM3 and galectin 9.

32. The method of any one of claims 25, wherein the immune checkpoint blocker is an antibody or antibody fragment targeting PD-1, PD-L1, CTLA-4, LAG3, TIM3, or a member of the B7 ligand family.

33. The method of any one of claims 1-23, further comprising administering an antagonist of VEGF.

34. The method of claim 33, wheren the antagonist of VEGF is an antibody or antibody fragment thereof that binds VEGF, an antibody or antibody fragment thereof that binds VEGF receptor, a small molecule inhibitor of the VEGF receptor tyrosine kinase, a dominant negative VEGF, or a VEGF receptor.

35. The method of any one of claims 1-32, wherein IL-2 or extended-PK IL-2, therapeutic antibody or fragment, cancer vaccine, and optional immune checkpoint blocker are administered simultaneously or sequentially.

36. The method of any one of claims 1-23 and 33-34, wherein IL-2 or extended-PK IL-2, therapeutic antibody or fragment, cancer vaccine, and optional antagonist of VEGF are administered simultaneously or sequentially.

37. The method of any one of claims 1-36, wherein the subject has a tumor.

38. The method of any one of claims 1-37, wherein the treatment increases the number of interferon gamma expressing CD8+ T cells in the tumor.

39. The method of any one of claims 1-38, wherein the treatment increases the ratio of CD8+ T cells to T regulatory cells in the tumor.

40. The method of any one of claims 1-39, wherein the hyperproliferative disorder is cancer.

41. The method of claim 40, wherein the cancer is selected from the group consisting of melanoma, leukemia, lymphoma, lung cancer, breast cancer, prostate cancer, ovarian cancer, colon cancer, mesothelioma, renal cell carcinoma, and brain cancer.

42. A method for inhibiting growth and/or proliferation of tumor cells in a subject comprising administering to the subject an effective amount of (i) IL-2 or extended-PK IL-2; (ii) a therapeutic antibody; and (iii) a cancer vaccine, thereby inhibiting growth and/or proliferation of tumor cells in the subject.

43. The method of claim 42, further comprising administering an immune checkpoint blocker.

44. The method of claim 42, further comprising administering an antagonist of VEGF.


SEQUENCE LISTING (50 sequences)

SEQ ID NO: 1 - protein, 330 aa, Homo sapiens [sequence omitted]

SEQ ID NO: 2 - protein, 232 aa, Homo sapiens
  misc_feature (1)..(232): Human IgG1 Fc domain (amino acid sequence)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 447 bp, Mouse
  misc_feature (1)..(447): Mouse IL-2 (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 4 - protein, 149 aa, Mouse
  misc_feature (1)..(149): Mouse IL-2 (amino acid sequence)
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 5 - DNA, 447 bp, Artificial Sequence
  Synthetic  QQ6210 (nucleic acid sequence)
gcacccacttcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcaa

SEQ ID NO: 6 - protein, 149 aa, Artificial Sequence
  Synthetic  QQ6210 (amino acid sequence)
APTSSSTSSSTAEAQQQQQQQQQQQQHLEQLLMDLQELLSRMEDHRNLRLPRMLTFKFYLPEQATELEDLQCLEDELEPLRQVLDLTQSKSFQLEDAENFISNIRVTVVKLKGSDNTFECQFDDEPATVVDFLRRWIAFCQSIISTSPQ

SEQ ID NO: 7 - DNA, 447 bp, Artificial Sequence
  Synthetic  E76A (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgctcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 8 - protein, 149 aa, Artificial Sequence
  Synthetic  E76A (amino acid sequence)
APTSSSTSSSTAEAQQQQQQQQQQQQHLEQLLMDLQELLSRMENYRNLKLPRMLTFKFYLPKQATELKDLQCLEDALGPLRHVLDLTQSKSFQLEDAENFISNIRVTVVKLKGSDNTFECQFDDESATVVDFLRRWIAFCQSIISTSPQ

SEQ ID NO: 9 - DNA, 447 bp, Artificial Sequence
  Synthetic  E76G (nucleic acid sequence)
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatggtcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 10 - protein, 149 aa, Artificial Sequence
  Synthetic  E76G (amino acid sequence)
APTSSSTSSSTAEAQQQQQQQQQQQQHLEQLLMDLQELLSRMENYRNLKLPRMLTFKFYLPKQATELKDLQCLEDGLGPLRHVLDLTQSKSFQLEDAENFISNIRVTVVKLKGSDNTFECQFDDESATVVDFLRRWIAFCQSIISTSPQ

SEQ ID NO: 11 - DNA, 816 bp, Artificial Sequence
  Synthetic  D265A Fc/Flag (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggtggcggatctgactacaaggacgacgatgacaagtgataa

SEQ ID NO: 12 - protein, 270 aa, Artificial Sequence
  Synthetic  D265A Fc/Flag (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSDYKDDDDK

SEQ ID NO: 13 - DNA, 1257 bp, Artificial Sequence
  Synthetic  D265A Fc/wt mIL-2 (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 14 - protein, 417 aa, Artificial Sequence
  Synthetic  D265A Fc/wt mIL-2 (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQHHHHHH

SEQ ID NO: 15 - DNA, 1257 bp, Artificial Sequence
  Synthetic  D265A Fc /QQ6210 (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcaacaccatcaccaccatcactgataa

SEQ ID NO: 16 - protein, 417 aa, Artificial Sequence
  Synthetic  D265A Fc /QQ6210 (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMEDHRNLRLPRMLTFKFYLPEQATELEDLQCLEDELEPLRQVLDLTQSKSFQLEDAENFISNIRVTVVKLKGSDNTFECQFDDEPATVVDFLRRWIAFCQSIISTSPQHHHHHH

SEQ ID NO: 17 - DNA, 1257 bp, Artificial Sequence
  Synthetic  D265A Fc / E76A (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgctcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 18 - protein, 417 aa, Artificial Sequence
  Synthetic  D265A Fc / E76A (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDALGPLRHVLDLTQSKSFQLEDAENFISNIRVTVVKLKGSDNTFECQFDDESATVVDFLRRWIAFCQSIISTSPQHHHHHH

SEQ ID NO: 19 - DNA, 1257 bp, Artificial Sequence
  Synthetic  D265A Fc / E76G (nucleic acid sequence)
atgagggtccccgctcagctcctggggctcctgctgctctggctcccaggtgcacgatgtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggccgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaaggagggggctccgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatggtcttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaacaccatcaccaccatcactgataa

SEQ ID NO: 20 - protein, 417 aa, Artificial Sequence
  Synthetic  D265A Fc / E76G (amino acid sequence)
MRVPAQLLGLLLLWLPGARCEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGGSAPTSSSTSSSTAEAQQQQQQQQQQQQHLEQLLMDLQELLSRMENYRNLKLPRMLTFKFYLPKQATELKDLQCLEDGLGPLRHVLDLTQSKSFQLEDAENFISNIRVTVVKLKGSDNTFECQFDDESATVVDFLRRWIAFCQSIISTSPQHHHHHH

SEQ ID NO: 21 - DNA, 447 bp, Artificial Sequence
  Synthetic  mIL-2 QQ 6.2-4 (nucleic acid sequence)
gcacccacctcaagctccacttcaagctctacagcggaagcacaacagcagcagcagcagcagcagcagcagcagcagcacctggagcagctgttgatggacctacaggagctcctgagcaggatggaggattccaggaacctgagactccccaggatgctcaccttcaaattttacttgcccaagcaggccacagaattggaagatcttcagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtgggctttctgaggagatggatagccttctgtcaaagcatcatctcaacgagccctcaa

SEQ ID NO: 22 - protein, 149 aa, Artificial Sequence
  Synthetic  mIL-2 QQ 6.2-4 (amino acid sequence)
APTSSSTSSSTAEAQQQQQQQQQQQQHLEQLLMDLQELLSRMEDSRNLRLPRMLTFKFYLPKQATELEDLQCLEDELEPLRQVLDLTQSKSFQLEDAENFISNIRVTVVKLKGSDNTFECQFDDEPATVVGFLRRWIAFCQSIISTSPQ

SEQ ID NO: 23 - DNA, 435 bp, Artificial Sequence
  Synthetic  mIL-2 QQ 6.2-8 (nucleic acid sequence)
gcacccacctcaagctccacttcaagctctacagcggaagcacaacagcagcagcagcagcagcagcacctggagcagctgttgatggacctacaggagctcctgagtaggatggaggatcacaggaacctgagactccccaggatgctcaccttcaaattttacttgcccaagcaggccacagaattggaagatcttcagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtggactttctgaggagatggatagccttctgtcaaagcatcatctcaacaagccctcga

SEQ ID NO: 24 - protein, 145 aa, Artificial Sequence
  Synthetic  mIL-2 QQ 6.2-8 (amino acid sequence)
APTSSSTSSSTAEAQQQQQQQQHLEQLLMDLQELLSRMEDHRNLRLPRMLTFKFYLPKQATELEDLQCLEDELEPLRQVLDLTQSKSFQLEDAENFISNIRVTVVKLKGSDNTFECQFDDEPATVVDFLRRWIAFCQSIISTSPR

SEQ ID NO: 25 - DNA, 447 bp, Artificial Sequence
  Synthetic  mIL-2 QQ 6.2-10 (nucleic acid sequence)
gcacccacctcaagctccacttcaagctctacagcggaagcacaacagcagcagcagcagcagcagcagcagcagcagcacctggagcagctgttgatggacctacaggaactcctgagtaggatggaggatcacaggaacctgagactccccaggatgctcaccttcaaattttacttgcccgagcaggccacagaattggaagatcttcagtgcctagaagatgaacttgaaccactgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcag

SEQ ID NO: 26 - protein, 149 aa, Artificial Sequence; identical to SEQ ID NO: 6
  Synthetic  mIL-2 QQ 6.2-10 (amino acid sequence)

SEQ ID NO: 27 - DNA, 438 bp, Artificial Sequence
  Synthetic  mIL-2 QQ 6.2-11 (nucleic acid sequence)
gcacccacctcaagctccacttcaagctctacagcggaagcacaacagcagcagcagcagcagcagcagcacctggagcagctgttgatggacctacaggagctcctgagcaggatggaggattccaggaacctgagactccccagaatgctcaccttcaaattttacttgcccgagcaggccacagaattgaaagatctccagtgcctagaagatgaacttgaacctctgcggcaagttctggatttgactcaaagcaaaagctttcaattggaagatgctgagaatttcatcagcaatatcagagtaactgttgtaaaactaaagggctctgacaacacatttgagtgccaattcgacgatgagccagcaactgtggtggactttctgaggagatggatagccttctgtcaaagcatcatctcaacaagccctcag

SEQ ID NO: 28 - protein, 146 aa, Artificial Sequence
  Synthetic  mIL-2 QQ 6.2-11 (amino acid sequence)
APTSSSTSSSTAEAQQQQQQQQQHLEQLLMDLQELLSRMEDSRNLRLPRMLTFKFYLPEQATELKDLQCLEDELEPLRQVLDLTQSKSFQLEDAENFISNIRVTVVKLKGSDNTFECQFDDEPATVVDFLRRWIAFCQSIISTSPQ

SEQ ID NO: 29 - DNA, 447 bp, Artificial Sequence
  Synthetic  mIL-2 QQ 6.2-13 (nucleic acid sequence)
gcacccacctcaagctccacttcaagctctacagcggaagcacaacagcagcagcagcagcagcagcagcagcagcagcacctggagcagctgttgatggacctacaggagctcctgagtaggatggaggatcacaggaacctgagactccccaggatgctcaccttcaaattttacttgcccgagcaggccacagaattgaaagatctccagtgcctagaagatgaacttgaacctctgcggcaggttctggatttgactcaaagcaaaagctttcaattggaagatgctgagaatttcatcagcaatatcagagtaactgttgtaaaactaaagggctctgacaacacatttgagtgccaattcgatgatgagccagcaactgtggtggactttctgaggagatggatagccttctgtcaaagcatcatctcaacaagccctcag

SEQ ID NO: 30 - protein, 149 aa, Artificial Sequence
  Synthetic  mIL-2 QQ 6.2-13 (amino acid sequence)
APTSSSTSSSTAEAQQQQQQQQQQQQHLEQLLMDLQELLSRMEDHRNLRLPRMLTFKFYLPEQATELKDLQCLEDELEPLRQVLDLTQSKSFQLEDAENFISNIRVTVVKLKGSDNTFECQFDDEPATVVDFLRRWIAFCQSIISTSPQ

SEQ ID NO: 31 - DNA, 461 bp, Homo sapiens
  misc_feature (1)..(461): Full length human IL-2 (nucleic acid sequence)
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtaatatgctgatgagacagcaaccattgtagaatttctgaacagatggattaccttttgtcaaagcatcatctcaacactgacttga

SEQ ID NO: 32 - protein, 153 aa, Homo sapiens
  misc_feature (1)..(153): Full length human IL-2 (amino acid sequence)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - DNA, 401 bp, Homo sapiens
  misc_feature (1)..(401): Human IL-2 without signal peptide (nucleic acid sequence)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtaatatgctgatgagacagcaaccattgtagaatttctgaacagatggattaccttttgtcaaagcatcatctcaacactgacttga

SEQ ID NO: 34 - protein, 133 aa, Homo sapiens [sequence omitted]

SEQ ID NO: 35 - protein, 749 aa, Homo sapiens
  misc_feature (1)..(749): Human serum albumin (amino acid sequence)
MDMRVPAQLLGLLLLWLPGARCADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 36 - protein, 726 aa, Homo sapiens
  misc_feature (1)..(726): Mature HSA (amino acid sequence)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 37 - DNA, 2247 bp, Homo sapiens
  misc_feature (1)..(2247): Human serum albumin (nucleic acid sequence)
atggatatgcgggtgcctgctcagctgctgggactgctgctgctgtggctgcctggggctagatgcgccgatgctcacaaaagcgaagtcgcacacaggttcaaagatctgggggaggaaaactttaaggctctggtgctgattgcattcgcccagtacctgcagcagtgcccctttgaggaccacgtgaaactggtcaacgaagtgactgagttcgccaagacctgcgtggccgacgaatctgctgagaattgtgataaaagtctgcatactctgtttggggataagctgtgtacagtggccactctgcgagaaacctatggagagatggcagactgctgtgccaaacaggaacccgagcggaacgaatgcttcctgcagcataaggacgataaccccaatctgcctcgcctggtgcgacctgaggtggacgtcatgtgtacagccttccacgataatgaggaaacttttctgaagaaatacctgtacgaaatcgctcggagacatccttacttttatgcaccagagctgctgttctttgccaaacgctacaaggccgctttcaccgagtgctgtcaggcagccgataaagctgcatgcctgctgcctaagctggacgaactgagggatgagggcaaggccagctccgctaaacagcgcctgaagtgtgctagcctgcagaaattcggggagcgagccttcaaggcttgggcagtggcacggctgagtcagagattcccaaaggcagaatttgccgaggtctcaaaactggtgaccgacctgacaaaggtgcacaccgaatgctgtcatggcgacctgctggagtgcgccgacgatcgagctgatctggcaaagtatatttgtgagaaccaggactccatctctagtaagctgaaagaatgctgtgagaaaccactgctggaaaagtctcactgcattgccgaagtggagaacgacgagatgccagctgatctgccctcactggccgctgacttcgtcgaaagcaaagatgtgtgtaagaattacgctgaggcaaaggatgtgttcctgggaatgtttctgtacgagtatgccaggcgccacccagactactccgtggtcctgctgctgaggctggctaaaacatatgaaaccacactggagaagtgctgtgcagccgctgatccccatgaatgctatgccaaagtcttcgacgagtttaagcccctggtggaggaacctcagaacctgatcaaacagaattgtgaactgtttgagcagctgggcgagtacaagttccagaacgccctgctggtgcgctataccaagaaagtcccacaggtgtccacacccactctggtggaggtgagccggaatctgggcaaagtggggagtaaatgctgtaagcaccctgaagccaagaggatgccatgcgctgaggattacctgagtgtggtcctgaatcagctgtgtgtcctgcatgaaaaaacacctgtcagcgaccgggtgacaaagtgctgtactgagtcactggtgaaccgacggccctgctttagcgccctggaagtcgatgagacttatgtgcctaaagagttcaacgctgagaccttcacatttcacgcagacatttgtaccctgagcgaaaaggagagacagatcaagaaacagacagccctggtcgaactggtgaagcataaacccaaggccacaaaagagcagctgaaggctgtcatggacgatttcgcagcctttgtggaaaaatgctgtaaggcagacgataaggagacttgctttgccgaggaaggaaagaaactggtggctgcatcccaggcagctctgggactgggaggaggatctgcccctacctcaagctccactaagaaaacccagctgcagctggagcacctgctgctggacctgcagatgattctgaacgggatcaacaattacaaaaatccaaagctgacccggatgctgacattcaagttttatatgcccaagaaagccacagagctgaaacacctgcagtgcctggaggaagagctgaagcctctggaagaggtgctgaacctggcccagagcaagaatttccatctgagaccaagggatctgatctccaacattaatgtgatcgtcctggaactgaagggatctgagactacctttatgtgcgaatacgctgacgagactgcaaccattgtggagttcctgaacagatggatcaccttctgccagtccatcatttctactctgacaggcggggggagc

SEQ ID NO: 38 - protein, 287 aa, Homo sapiens
  misc_feature (1)..(287): PD-1
MQIPQAPWPVVWAVLQLGWRPGWFLDSPDPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 39 - protein, 290 aa, Homo sapiens
  misc_feature (1)..(290): PD-L-1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 40 - protein, 223 aa, Homo sapiens
  misc_feature (1)..(223): CTLA-4
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 41 - protein, 525 aa, Homo sapiens
  misc_feature (1)..(525): LAG3
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 42 - protein, 300 aa, Homo sapiens
  misc_feature (1)..(300): TIM3
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 43 - protein, 532 aa, Homo sapiens
  misc_feature (1)..(532): B7-H3
MLRRRGSPGMGVHVGAALGALWFCLTGALEVQVPEDPVVALVGTDATLCCSFSPEPGFSLQLNLIWQLTDTKQLVHSFAEGQDQGSAYANRTALFPDLLAQGNASLRLQRVRVADEGSFCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 44 - protein, 166 aa, Homo sapiens
  misc_feature (1)..(166): B7-H4
MASLGQILFWSIISIIIILAGAIALIIGFGISAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 45 - protein, 51 aa, Artificial Sequence
  Synthetic  peptide linker
SGGGGSGGGGSGGGGSGGGGSGGGGSGGGGSGGGGSGGGGSGGGGSGGGGS

SEQ ID NO: 46 - protein, 16 aa, Artificial Sequence
  Synthetic  peptide linker
SGGGGSGGGGSGGGGS

SEQ ID NO: 47 - protein, 21 aa, Artificial Sequence
  Synthetic  peptide linker
SGGGGSGGGGSGGGGSGGGGS

SEQ ID NO: 48 - protein, 30 aa, Artificial Sequence
  Synthetic  peptide linker
GGGGSGGGGSGGGGSGGGGSGGGGSGGGGS

SEQ ID NO: 49 - protein, 24 aa, Artificial Sequence
  Synthetic  peptide linker
GGGSGGGSGGGSGGGSGGGSGGGS

SEQ ID NO: 50 - DNA, 25 bp, Artificial Sequence
  Synthetic lipophilic-CpG oligonucleotide
gggggtccatgacgttcctgacgtt